(19) United States
(12) Patent Application Publication
(10) Pub. No.: US 2012/0316105 A1
(43) Pub. Date: Dec. 13, 2012

(54) Polymyxin Derivates Useful As Antibacterial Agents

(75) Inventors: Thomas Victor Magee, Winchester, MA (US); Jinshan Michael Chen, Madison, CT (US); Carlos Alberto Martinez, Quaker Hill, CT (US); Zhengong B. Li, East Lyme, CT (US)

(73) Assignee: Pfizer Inc.

(21) Appl. No.: 13/490,887

(22) Filed: Jun. 7, 2012

Related U.S. Application Data
(60) Provisional application No. 61/494,645, filed on Jun. 8, 2011.

(51) Int. Cl. A61K 38/12; A61P 31/04; C07K 7/62

(52) U.S. Cl. 514/2.8; 530/319; 514/21.1; 514/2.9

(57) ABSTRACT

The present invention provides a new class of polymyxin derivates useful for treating bacterial infections, especially Gram-negative infections, that have reduced renal cytotoxicity.

### FIELD OF THE INVENTION

This invention relates to a new class of polymyxin derivatives with potent antibacterial activity and reduced renal toxicity. The invention also relates to methods of using such compounds in the treatment of bacterial infections (especially Gram-negative infections) and to pharmaceutical compositions containing such compounds.

### BACKGROUND OF THE INVENTION

Major therapeutic problems are caused by multidrug resistant (MDR) Gram-negative bacteria such as Pseudomonas aeruginosa, Acinetobacter baumannii, and Klebsiella pneumoniae especially in the case of hospital-acquired infections. For example, in 2002, 33% of Pseudomonas aeruginosa infections from intensive care units were resistant to fluoroquinolones, while resistance to imipenem was 22% (Clin. Infect. Dis. 42: 657-68, 2006). In addition, multi drug-resistant (MDR) infections are also increasing; in the case of Pseudomonas aeruginosa, MDR increased from 4% in 1992 to 14% in 2002 (Biochem Pharm 71: 991, 2006).

The polymyxins are one of the few classes of antibiotics currently used to treat the major pathogens of interest, namely P. aeruginosa, K. pneumoniae, and A. baumannii. Polymyxins are mostly used as a last-line antibiotic for otherwise untreatable serious infections. The major limitation associated with polymyxin is renal toxicity which may complicate a patient's therapy or even require its discontinuation. The present invention provides polymyxin derivates that have potent activity against MDR Gram-negative bacteria and reduced renal cytotoxicity.

### SUMMARY OF THE INVENTION

A new class of polymyxin derivatives has been discovered. These compounds, or their pharmaceutically acceptable salts, are represented by Formula I below:

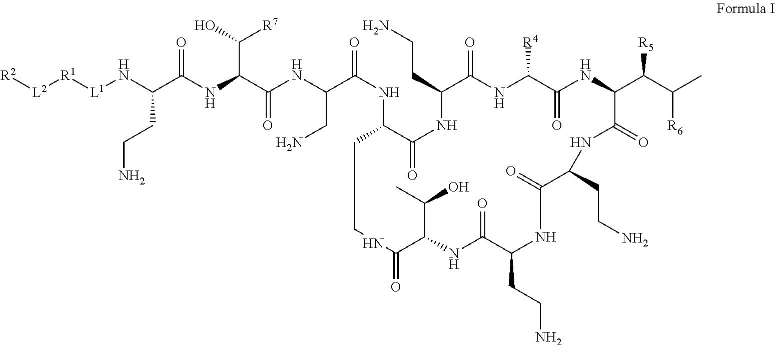

or a pharmaceutically acceptable salt thereof
wherein

L¹ is a bond, carbonyl, sulfonyl, —NHC(O)—, or —OC(O)—;

R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;

L² is absent, a bond, —(CH₂)ₙ—, —(CH₂)ₙ—O—(CH₂)ₚ—, —(CH₂)ₙS(CH₂)ₚ—, or —(CH₂)ₙNR³(CH₂)ₚ—;

R² is absent, (C₆-C₁₀)aryl, or (C₅-C₁₂)heteroaryl;

R³ is H or (C₁-C₃)alkyl;

R⁴ is (C₁-C₆)alkyl or (C₆-C₁₀)aryl(C₁-C₆)alkyl;

R⁵ is H or CH₃;

R⁶ is H or CH₃;

wherein R⁶ is H when R⁵ is CH₃ and wherein R⁶ is CH₃ when R⁵ is H;

R⁷ is H or CH₃;

n is 0, 1, 2, or 3; and

p is 0, 1, 2, or 3.

The compounds of Formula I exhibit antibacterial activity, especially against Gram-negative organisms and are useful for treating bacterial infections in mammals, especially humans. The compounds are also useful for veterinary applications, such as treating infections in livestock and companion animals.

The compounds of Formula I are useful for treating a variety of infections; especially Gram-negative infections associated with P. aeruginosa, K. pneumoniae, and A. baumannii including nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, and central nervous system infections.

In order to simplify administration, the compounds will typically be admixed with at least one excipient and formulated into a pharmaceutical dosage form. Examples of such dosage forms include tablets, capsules, solutions/suspensions for injection, aerosols for inhalation, cream/ointments for topical, otic or ophthalmic use, and solutions/suspensions for oral ingestion.

### DETAILED DESCRIPTION OF THE INVENTION

Pharmaceutical grade polymyxin B (PMB) is composed mainly of two structurally related compounds, polymyxin B1 (PMB1) and polymyxin B2 (PMB2). The structural differences between PMB1 and PMB2 reside in the fatty acid portion of the PMB template. The fatty acid chain for PMB1, shown below, is composed of a (S)-6-methyloctanoyl group whereas the PMB2 fatty acid chain has one less carbon or a 6-methylheptanoyl group.

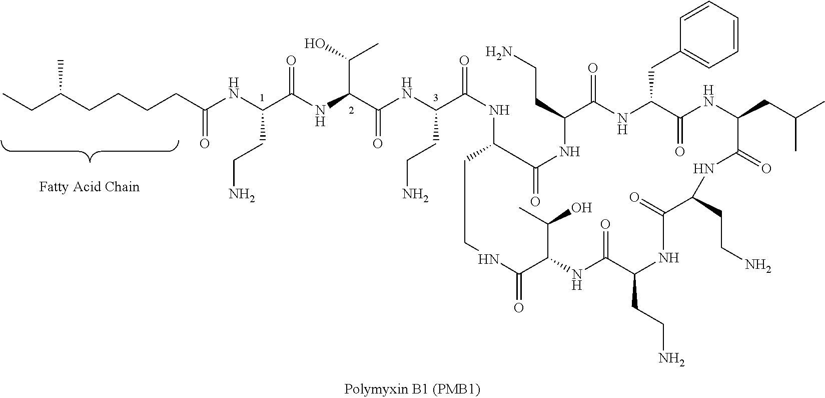

The present invention provides PMB derivatives with reduced cytotoxicity and improved activity against MDR Gram-negative bacteria as compared to PMB. The structural difference consistent with all the compounds of Formula I is that the amino acid (S)-2,4-diaminobutanoic acid (Dab) at the three position of the PMB template is replaced with (S)-2,3-diaminopropanoic acid (Dap). The present invention also includes compounds where the fatty acid chain is replaced with aryl or heteroaryl groups. The replacement of Dab with Dap, alone in or in combination with replacement of the fatty acid chain, unexpectedly provides antibacterial agents of Formula I with reduced cytotoxicity in human renal cells and improved antibacterial activity against P. aeruginosa, K. pneumoniae, and/or A. baumannii.

In one embodiment, the present invention provides compounds of Formula II

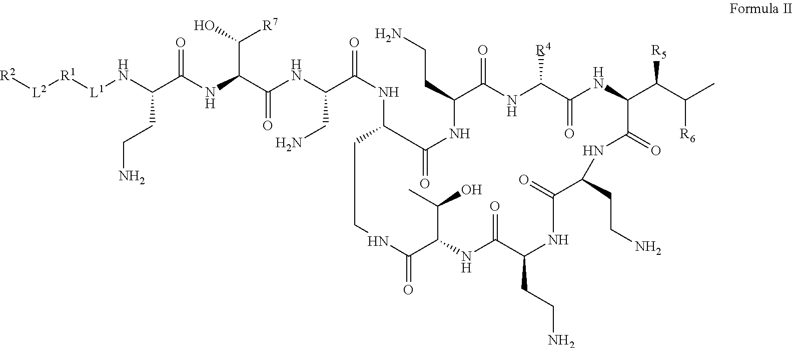

or a pharmaceutically acceptable salt thereof
wherein

L¹ is a bond, carbonyl, sulfonyl, —NHC(O)—, or —OC(O)—;

R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;

L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;

R² is absent, (C₆-C₁₀)aryl, or (C₅-C₁₂)heteroaryl;

R³ is H or (C₁-C₃)alkyl;

R⁴ is (C₁-C₆)alkyl or (C₆-C₁₀)aryl(C₁-C₆)alkyl;

R⁵ is H or CH₃;

R⁶ is H or CH₃;

wherein R⁶ is H when R⁵ is CH₃ and wherein R⁶ is CH₃ when R⁵ is H;

R⁷ is H or CH₃;

n is 0, 1, 2, or 3; and

p is 0, 1, 2, or 3.

In another embodiment, the present invention provides compounds of Formula II, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, (C₆-C₁₀)aryl(C₂-C₄)alkenyl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0; and p is 0.

In one embodiment, the present invention provides compounds of Formula II, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0; and p is 0.

In another embodiment, the present invention provides compounds of Formula II, or a pharmaceutically acceptable salt thereof, wherein L¹ is carbonyl; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is carbonyl; R¹ is (C₁-C₁₀)alkyl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₁-C₁₀)alkyl; L² is a bond; R² is phenyl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₁-C₁₀)alkyl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₁-C₁₀)alkyl; L² is a bond; R² is phenyl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₁-C₁₀)alkyl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₁-C₁₀)alkyl; L² is a bond; R² is phenyl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₁-C₁₀)alkyl; L² is absent; R² is absent; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₁-C₁₀)alkyl; L² is a bond; R² is phenyl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₆-C₁₀)aryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is carbonyl; R¹ is (C₆-C₁₀)aryl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₆-C₁₀)aryl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is H; R⁶ is CH₃ and R⁷ is CH₃.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₆-C₁₀)aryl; L² is absent; R² is absent; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₆-C₁₀)aryl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is H; R⁶ is CH₃ and R⁷ is H.

In another embodiment, the present invention provides compounds of Formula II, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is carbonyl; R¹ is (C₅-C₁₂)heteroaryl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is pyridinyl; L² is a bond; R² is phenyl optionally substituted with Cl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₅-C₁₂)heteroaryl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is pyridinyl; L² is a bond; R² is phenyl optionally substituted with Cl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₅-C₁₂)heteroaryl; L² is absent; R² is absent; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is pyridinyl; L² is a bond; R² is phenyl optionally substituted with Cl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is carbonyl; R¹ is (C₅-C₁₂)heteroaryl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is pyridinyl; L² is a bond; R² is phenyl optionally substituted with Cl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₅-C₁₂)heteroaryl; L² is —(CH₂)ₙO(CH₂)ₚ—; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is H; R⁶ is CH₃; R⁷ is CH₃; n is 0; and p is 0.

In another embodiment, the present invention provides compounds of Formula II, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is H; R⁶ is CH₃; R⁷ is H; n is 0; and p is 0.

In another embodiment, the present invention provides compounds of Formula II, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is CH₃; R⁶ is H; R⁷ is H; n is 0; and p is 0.

In another embodiment, the present invention provides compounds of Formula II, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is CH₃; R⁶ is H; R⁷ is CH₃; n is 0; and p is 0.

In another embodiment, the present invention provides compounds of Formula II, or a pharmaceutically acceptable salt thereof, wherein L¹ is —NHC(O)—; R¹ is (C₆-C₁₀)aryl; L² is a bond; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; and R⁷ is H or CH₃.

In another embodiment, the present invention provides compounds of Formula II, or a pharmaceutically acceptable salt thereof, wherein L¹ is —NHC(O)—; R¹ is phenyl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is —NHC(O)—; R¹ is phenyl; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is —NHC(O)—; R¹ is phenyl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is —NHC(O)—; R¹ is phenyl; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ sulfonyl; R¹ is (C₆-C₁₀)aryl; L² is a bond; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; and R⁷ is H or CH₃.

In another embodiment, the present invention provides compounds of Formula II, or a pharmaceutically acceptable salt thereof, wherein L¹ is sulfonyl; R¹ is phenyl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is sulfonyl; R¹ is phenyl; L² is a bond; R² is (C₆-C₁₀)aryl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is sulfonyl; R¹ is phenyl; L² is a bond; R² is (C₆-C₁₀)aryl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is sulfonyl; R¹ is phenyl; L² is a bond; R² is (C₆-C₁₀)aryl; R⁴ is benzyl; R⁵ is H; R⁶ is CH₃; and R⁷ is CH₃.

In another embodiment, the present invention provides a compound of Formula II that is

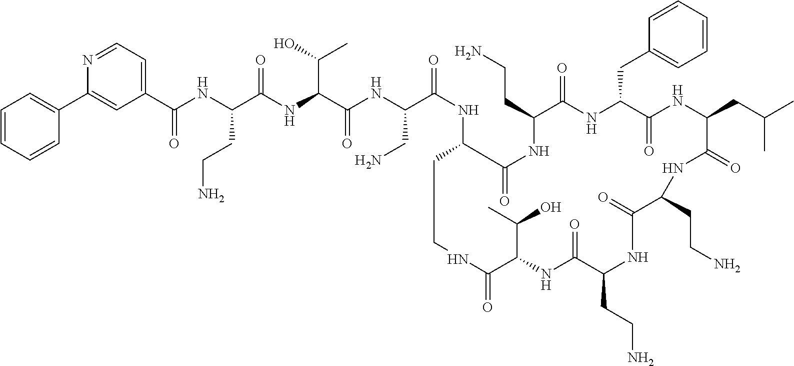

or a pharmaceutically acceptable salt thereof.

In another embodiment, the present invention provides a compound of Formula II that is

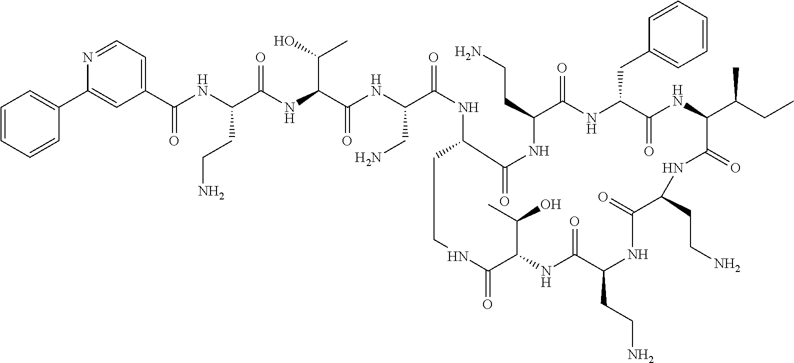

or a pharmaceutically acceptable salt thereof.

In another embodiment, the present invention provides a compound of Formula II that is

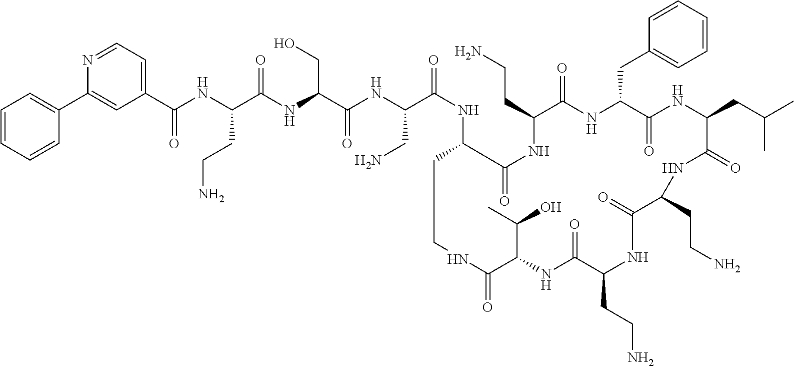

or a pharmaceutically acceptable salt thereof.

In another embodiment, the present invention provides a compound of Formula II that is

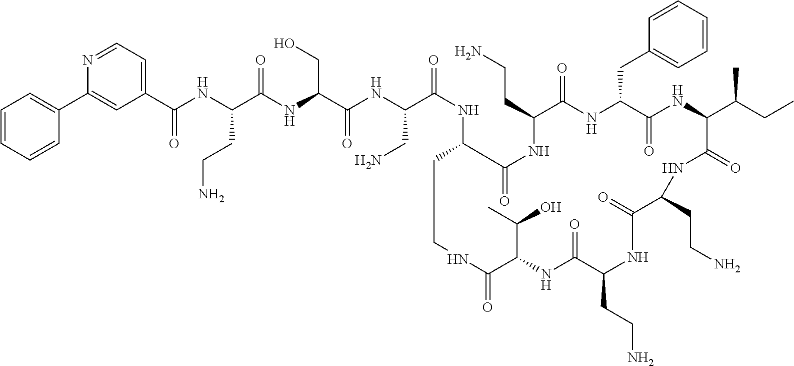

or a pharmaceutically acceptable salt thereof.

In another embodiment, the present invention provides a compound of Formula II that is

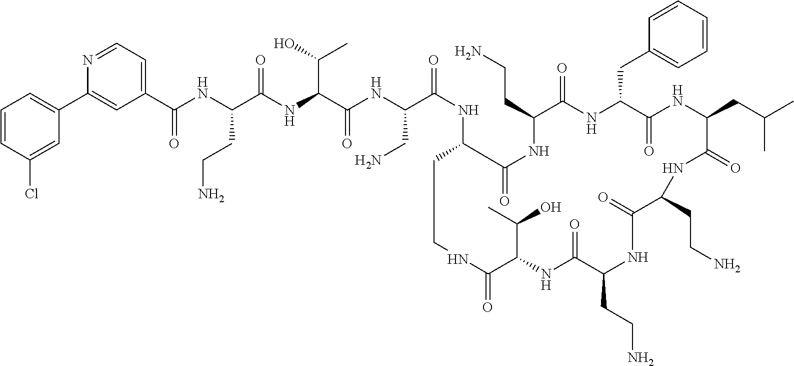

or a pharmaceutically acceptable salt thereof.

In another embodiment, the present invention provides a compound of Formula II that is

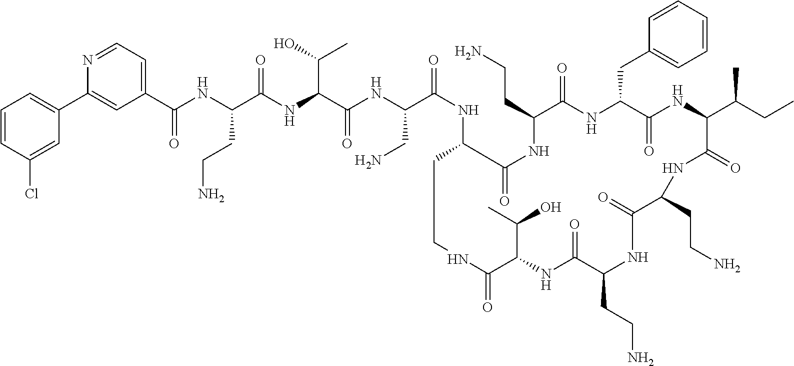

or a pharmaceutically acceptable salt thereof.

In another embodiment, the present invention provides a compound of Formula II that is

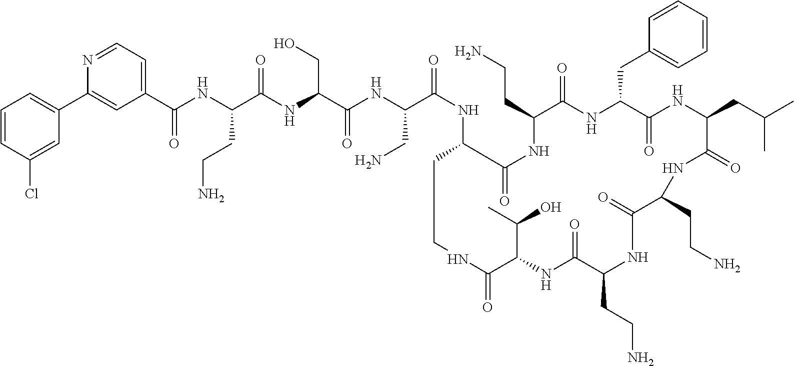

or a pharmaceutically acceptable salt thereof.

In another embodiment, the present invention provides a compound of Formula II that is

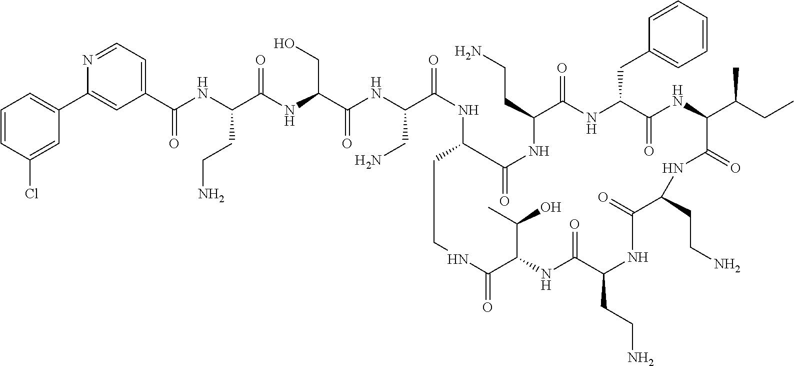

or a pharmaceutically acceptable salt thereof.

In another embodiment, the present invention provides compounds of Formula II, or a pharmaceutically acceptable salt thereof, wherein L¹ is —OC(O)—; R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is —OC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is —OC(O)—; R¹ is (C₅-C₁₂)heteroaryl; L² is a bond; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃.

In another embodiment, the present invention provides compounds of Formula II, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is H.

In another embodiment, the present invention provides compounds of Formula II, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₆-C₁₀)aryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula II, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is H; R⁶ is CH₃; and R⁷ is CH₃.

In another embodiment, the present invention provides compounds of Formula II, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is CH₃; R⁶ is H; and R⁷ is CH₃.

In another embodiment, the present invention provides compounds of Formula II, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is H; R⁶ is CH₃; and R⁷ is H.

In another embodiment, the present invention provides compounds of Formula II, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is CH₃; R⁶ is H; and R⁷ is H.

In another embodiment, the present invention provides a pharmaceutical composition comprising a compound of Formula II, or a pharmaceutically acceptable salt thereof, in admixture with at least one pharmaceutically acceptable excipient.

In another embodiment, the present invention provides a method for treating bacterial infections in a mammal, in particular humans, comprising administering to the mammal in need of such treatment a therapeutically effect amount of a compound of Formula II, or a pharmaceutically acceptable salt thereof. The methods of the present invention are particularly useful for treating Gram-negative bacterial infections including, but not limited to, infections associated with Pseudomonas aeruginosa, Acinetobacter baumannii, and Klebsiella pneumoniae.

In another embodiment, the present invention provides a method for treating nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, or central nervous system infections comprising administering to the mammal, particularly a human, in need of such treatment a therapeutically effect amount of a compound of Formula II, or a pharmaceutically acceptable salt thereof.

In another embodiment, the present invention provides the use of a compound of Formula II, or a pharmaceutically acceptable salt thereof, in the manufacture of a medicament for treating bacterial infections, particularly Gram-negative bacterial infections.

In another embodiment, the present invention provides combinations comprising a first compound of Formula I wherein R⁵ is H, R⁶ is CH₃ (leucine derivative) and a second compound of Formula I wherein R⁵ is CH₃ and R⁶ is H (isoleucine derivative). The ratio of the first compound to the second compound can be 99:1 to 1:99. A preferred ratio of the first compound (leucine derivative) to the second compound (isoleucine derivative) is 80:20 to 99:1. The most preferred ratio is 90:10 to 99:1.

In another embodiment, the present invention provides a combination comprising any one of Examples 1-47 (leucine derivatives) in combination with its corresponding isoleucine derivative in a ratio of 99:1 to 1:99, preferably the ratio is 80:20 to 99:1, most preferably the ratio is 90:10 to 99:1.

In another embodiment, the present invention provides a combination comprising Example 1 and Example 49 in a ratio of 99:1 to 1:99, preferably the ratio is 80:20 to 99:1, most preferably the ratio is 90:10 to 99:1.

In another embodiment, the present invention provides a combination comprising Example 3 and Example 48 in a ratio of 99:1 to 1:99, preferably in a ratio of 80:20 to 99:1, most preferably in a ratio of 90:10 to 99:1. The corresponding isoleucine compound of Example 3 is a compound of Formula I where L¹ is carbonyl, R¹ is pyridin-4-yl, L² is a bond, R² is 3-chlorophenyl attached to the 2-position of pyridin-4-yl, R⁴ is benzyl, R⁵ is CH₃, and R⁶ is H.

In another embodiment, the present invention provides a pharmaceutical composition comprising a combination, or a pharmaceutically acceptable salt thereof, in admixture with at least one pharmaceutically acceptable excipient.

In another embodiment, the present invention provides a method for treating bacterial infections in a mammal, in particular humans, comprising administering to the mammal in need of such treatment a therapeutically effect amount of a combination, or a pharmaceutically acceptable salt thereof. The methods of the present invention are particularly useful for treating Gram-negative bacterial infections including, but not limited to, infections associated with Pseudomonas aeruginosa, Acinetobacter baumannii, and Klebsiella pneumoniae.

In another embodiment, the present invention provides a method for treating nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, or central nervous system infections comprising administering to the mammal, particularly a human, in need of such treatment a therapeutically effect amount of a combination, or a pharmaceutically acceptable salt thereof.

In one embodiment, the present invention provides compounds of Formula III

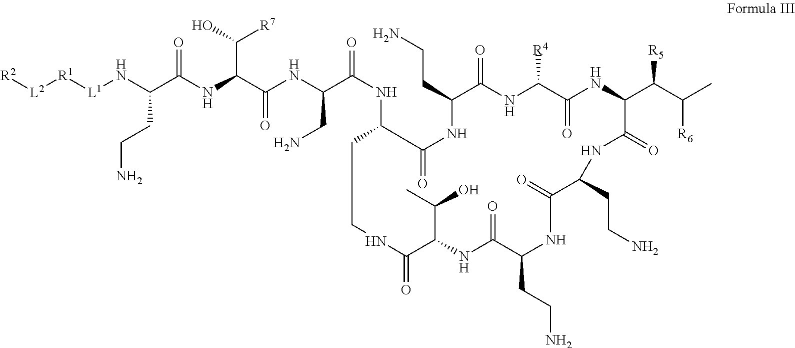

or a pharmaceutically acceptable salt thereof
wherein

L¹ is a bond, carbonyl, sulfonyl, —NHC(O)—, or —OC(O)—;

R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;

L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;

R² is absent, (C₆-C₁₀)aryl, or (C₅-C₁₂)heteroaryl;

R³ is H or (C₁-C₃)alkyl;

R⁴ is (C₁-C₆)alkyl or (C₆-C₁₀)aryl(C₁-C₆)alkyl;

R⁵ is H or CH₃;

R⁶ is H or CH₃;

wherein R⁶ is H when R⁵ is CH₃ and wherein R⁶ is CH₃ when R⁵ is H;

R⁷ is H or CH₃;

n is 0, 1, 2, or 3; and

p is 0, 1, 2, or 3.

In another embodiment, the present invention provides compounds of Formula III, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, (C₆-C₁₀)aryl(C₂-C₄)alkenyl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0; and p is 0.

In one embodiment, the present invention provides compounds of Formula III, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0; and p is 0.

In another embodiment, the present invention provides compounds of Formula III, or a pharmaceutically acceptable salt thereof, wherein L¹ is carbonyl; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is carbonyl; R¹ is (C₁-C₁₀)alkyl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₁-C₁₀)alkyl; L² is a bond; R² is phenyl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₁-C₁₀)alkyl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₁-C₁₀)alkyl; L² is a bond; R² is phenyl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₁-C₁₀)alkyl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₁-C₁₀)alkyl; L² is a bond; R² is phenyl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₁-C₁₀)alkyl; L² is absent; R² is absent; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₁-C₁₀)alkyl; L² is a bond; R² is phenyl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₆-C₁₀)aryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is carbonyl; R¹ is (C₆-C₁₀)aryl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₆-C₁₀)aryl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is H; R⁶ is CH₃ and R⁷ is CH₃.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₆-C₁₀)aryl; L² is absent; R² is absent; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₆-C₁₀)aryl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is H; R⁶ is CH₃ and R⁷ is H.

In another embodiment, the present invention provides compounds of Formula III, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is carbonyl; R¹ is (C₅-C₁₂)heteroaryl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is pyridinyl; L² is a bond; R² is phenyl optionally substituted with Cl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₅-C₁₂)heteroaryl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is pyridinyl; L² is a bond; R² is phenyl optionally substituted with Cl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₅-C₁₂)heteroaryl; L² is absent; R² is absent; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is carbonyl; R¹ is pyridinyl; L² is a bond; R² is phenyl optionally substituted with Cl; R⁴ is benzyl; R⁵ is CH₃; R⁶ is H; and R⁷ is H.

In another embodiment, the present invention provides compounds of Formula IIII, or a pharmaceutically acceptable salt thereof, wherein L¹ is carbonyl; R¹ is (C₅-C₁₂)heteroaryl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is pyridinyl; L² is a bond; R² is phenyl optionally substituted with Cl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₅-C₁₂)heteroaryl; L² is —(CH₂)ₙO(CH₂)ₚ—; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is H; R⁶ is CH₃; R⁷ is CH₃; n is 0; and p is 0.

In another embodiment, the present invention provides compounds of Formula III, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is H; R⁶ is CH₃; R⁷ is H; n is 0; and p is 0.

In another embodiment, the present invention provides compounds of Formula III, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is CH₃; R⁶ is H; R⁷ is H; n is 0; and p is 0.

In another embodiment, the present invention provides compounds of Formula III, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is CH₃; R⁶ is H; R⁷ is CH₃; n is 0; and p is 0.

In another embodiment, the present invention provides compounds of Formula III, or a pharmaceutically acceptable salt thereof, wherein L¹ is —NHC(O)—; R¹ is (C₆-C₁₀)aryl; L² is a bond; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; and R⁷ is H or CH₃.

In another embodiment, the present invention provides compounds of Formula III, or a pharmaceutically acceptable salt thereof, wherein L¹ is —NHC(O)—; R¹ is phenyl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is —NHC(O)—;

R¹ is phenyl; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is —NHC(O)—; R¹ is phenyl; L² is a bond; R² is (C₆-C₁₀)aryl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is —NHC(O)—; R¹ is phenyl; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ sulfonyl; R¹ is (C₆-C₁₀)aryl; L² is a bond; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; and R⁷ is H or CH₃.

In another embodiment, the present invention provides compounds of Formula III, or a pharmaceutically acceptable salt thereof, wherein L¹ is sulfonyl; R¹ is phenyl; L² is a bond; R² is (C₆-C₁₀)aryl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is sulfonyl; R¹ is phenyl; L² is a bond; R² is (C₆-C₁₀)aryl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is sulfonyl; R¹ is phenyl; L² is a bond; R² is (C₆-C₁₀)aryl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is sulfonyl; R¹ is phenyl; L² is a bond; R² is (C₆-C₁₀)aryl; R⁴ is benzyl; R⁵ is H; R⁶ is CH₃; and R⁷ is CH₃.

In another embodiment, the present invention provides a compound of Formula III that is

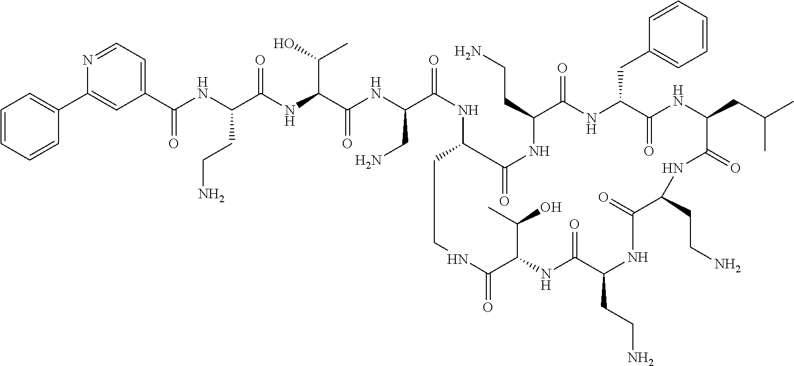

or a pharmaceutically acceptable salt thereof.

In another embodiment, the present invention provides a compound of Formula III that is

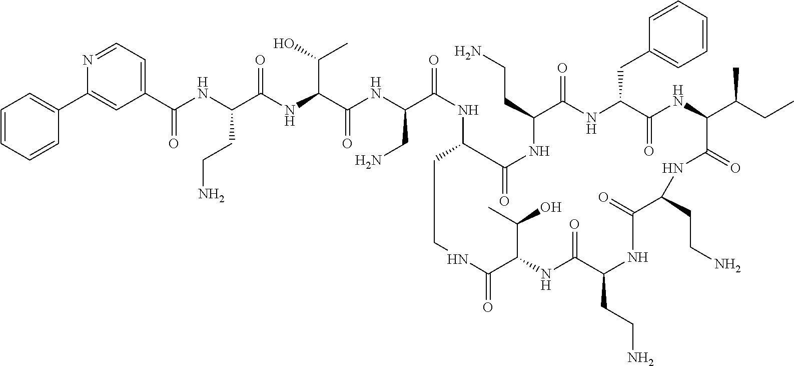

or a pharmaceutically acceptable salt thereof.

In another embodiment, the present invention provides a compound of Formula III that is

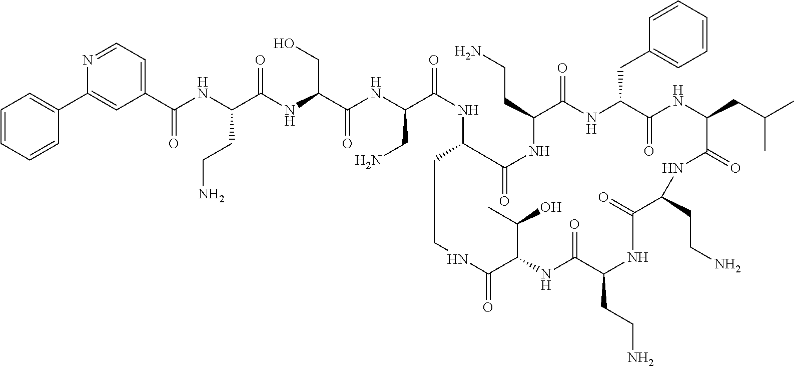

or a pharmaceutically acceptable salt thereof.

In another embodiment, the present invention provides a compound of Formula III that is

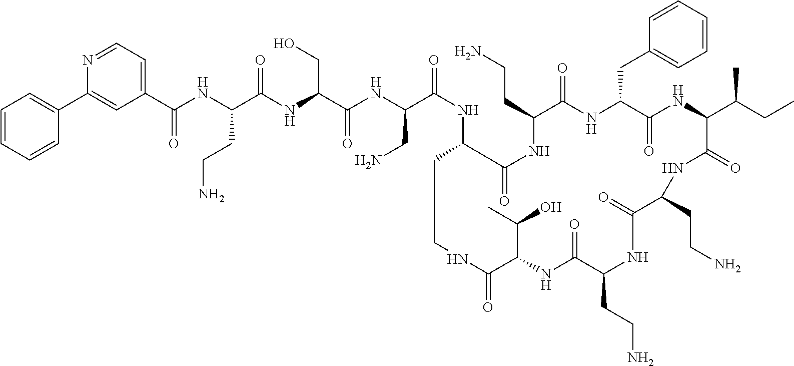

or a pharmaceutically acceptable salt thereof.

In another embodiment, the present invention provides a compound of Formula III that is

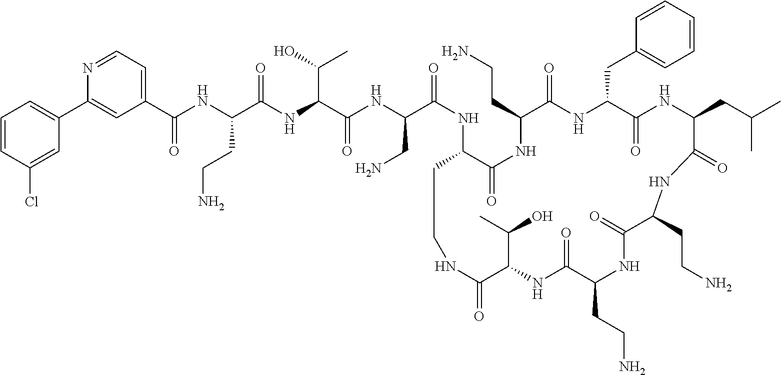

or a pharmaceutically acceptable salt thereof.

In another embodiment, the present invention provides a compound of Formula III that is

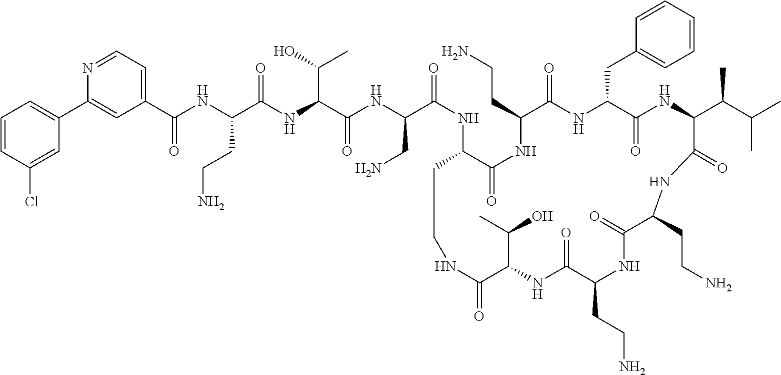

or a pharmaceutically acceptable salt thereof.

In another embodiment, the present invention provides a compound of Formula III that is

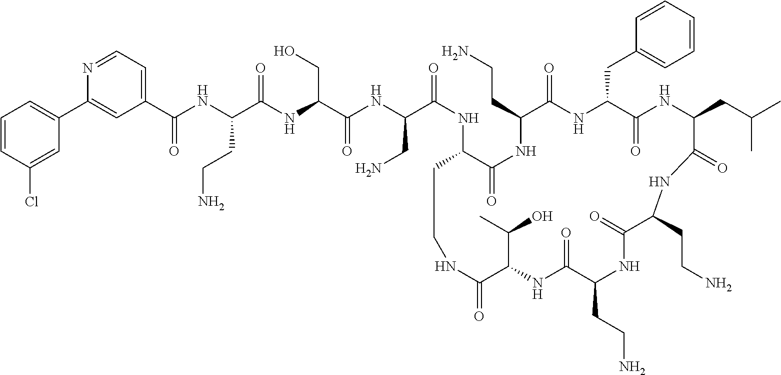

or a pharmaceutically acceptable salt thereof.

In another embodiment, the present invention provides a compound of Formula III that is

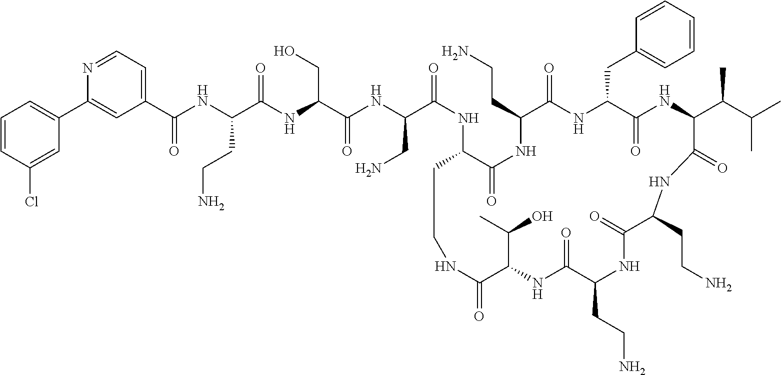

or a pharmaceutically acceptable salt thereof.

In another embodiment, the present invention provides compounds of Formula III, or a pharmaceutically acceptable salt thereof, wherein L¹ is —OC(O)—; R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is —OC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is —OC(O)—; R¹ is (C₅-C₁₂)heteroaryl; L² is a bond; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃.

In another embodiment, the present invention provides compounds of Formula III, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; and R⁷ is H.

In another embodiment, the present invention provides compounds of Formula III, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₆-C₁₀)aryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; n is 0, 1, 2, or 3; and p is 0, 1, 2, or 3.

In another embodiment, the present invention provides compounds of Formula III, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is H; R⁶ is CH₃; and R⁷ is CH₃.

In another embodiment, the present invention provides compounds of Formula III, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is CH₃; R⁶ is H; and R⁷ is CH₃.

In another embodiment, the present invention provides compounds of Formula III, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is H; R⁶ is CH₃; and R⁷ is H.

In another embodiment, the present invention provides compounds of Formula III, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is CH₃; R⁶ is H; and R⁷ is H.

In another embodiment, the present invention provides a pharmaceutical composition comprising a compound of Formula III, or a pharmaceutically acceptable salt thereof, in admixture with at least one pharmaceutically acceptable excipient.

In another embodiment, the present invention provides a method for treating bacterial infections in a mammal, in particular humans, comprising administering to the mammal in need of such treatment a therapeutically effect amount of a compound of Formula III, or a pharmaceutically acceptable salt thereof. The methods of the present invention are particularly useful for treating Gram-negative bacterial infections including, but not limited to, infections associated with Pseudomonas aeruginosa, Acinetobacter baumannii, and Klebsiella pneumoniae.

In another embodiment, the present invention provides a method for treating nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, or central nervous system infections comprising administering to the mammal, particularly a human, in need of such treatment a therapeutically effect amount of a compound of Formula III, or a pharmaceutically acceptable salt thereof.

In another embodiment, the present invention provides the use of a compound of Formula III, or a pharmaceutically acceptable salt thereof, in the manufacture of a medicament for treating bacterial infections, particularly Gram-negative bacterial infections.

In another embodiment, the present invention provides a compound of Formula IV

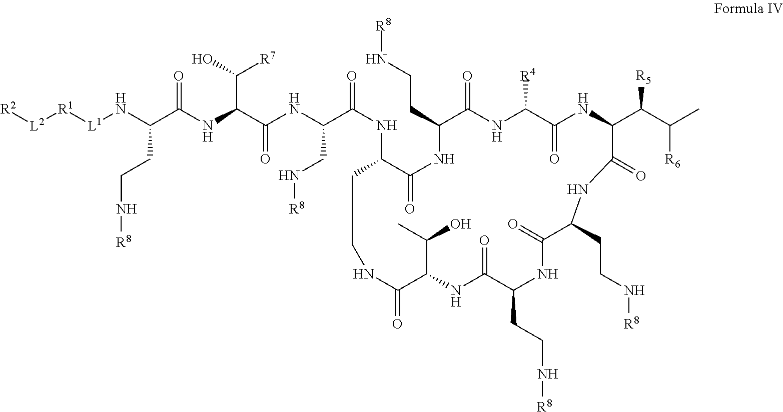

or a pharmaceutically acceptable salt thereof
wherein

L¹ is a bond, carbonyl, sulfonyl, —NHC(O)—, or —OC(O)—;

R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;

L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;

R² is absent, (C₆-C₁₀)aryl, or (C₅-C₁₂)heteroaryl;

R³ is H or (C₁-C₃)alkyl;

R⁴ is (C₁-C₆)alkyl or (C₆-C₁₀)aryl(C₁-C₆)alkyl;

R⁵ is H or CH₃;

R⁶ is H or CH₃;

wherein R⁶ is H when R⁵ is CH₃ and wherein R⁶ is CH₃ when R⁵ is H;

R⁷ is H or CH₃;

R⁸ is (C₁-C₁₀)alkylcarbonyl, (C₁-C₁₀)alkyl-O-carbonyl, (C₆-C₁₀)aryl-O-carbonyl, or —CH₂—SO₂OH;

n is 0, 1, 2, or 3; and

p is 0, 1, 2, or 3.

In one embodiment, the present invention provides compounds of Formula IV, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₆-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₆-C₁₂)heteroaryl; R³ is H or (C₁-C₃)alkyl; R⁴ is (C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₆-C₁₂)heteroaryl; R³ is H or (C₁-C₃)alkyl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n iso, 1, 2, or 3; and p is 0, 1, 2, or 3.

In another embodiment, the present invention provides compounds of Formula IV, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0; and p is 0.

In one embodiment, the present invention provides compounds of Formula IV, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₆-C₁₂)heteroaryl; R³ is H or (C₁-C₃)alkyl; R⁴ is (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0; and p is 0.

In another embodiment, the present invention provides compounds of Formula IV, or a pharmaceutically acceptable salt thereof, wherein L¹ is carbonyl; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is carbonyl; R¹ is (C₁-C₁₀)alkyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₁-C₁₀)alkyl; L² is a bond; R² is phenyl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₁-C₁₀)alkyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₁-C₁₀)alkyl; L² is a bond; R² is phenyl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is carbonyl; R¹ is (C₆-C₁₀)aryl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is CH₃; R⁷ is CH₃; R⁶ is H; and R⁸ is —CH₂—SO₂OH.

In another embodiment, the present invention provides compounds of Formula IV, or a pharmaceutically acceptable salt thereof, wherein L¹ is carbonyl; R¹ is (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH n is 0, 1, 2, or 3; and p is 0, 1, 2, or 3.

In another embodiment, the present invention provides compounds of Formula IV, or a pharmaceutically acceptable salt thereof, wherein L¹ is carbonyl; R¹ is (C₅-C₁₂)heteroaryl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₅-C₁₂)heteroaryl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₅-C₁₂)heteroaryl; L² is —(CH₂)ₙO(CH₂)ₚ—; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is CH₃; R⁸ is —CH₂—SO₂OH; n is 0; and p is 0.

In another embodiment, the present invention provides compounds of Formula IV, or a pharmaceutically acceptable salt thereof, wherein L¹ is —NHC(O)—; R¹ is (C₆-C₁₀)aryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is —NHC(O)—; R¹ is phenyl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ sulfonyl; R¹ is (C₆-C₁₀)aryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is sulfonyl; R¹ is phenyl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is —NHC(O)—; R¹ is (C₆-C₁₀)aryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is —NHC(O)—; R¹ is phenyl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ sulfonyl; R¹ is (C₆-C₁₀)aryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is sulfonyl; R¹ is phenyl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides a compound of Formula IV that is

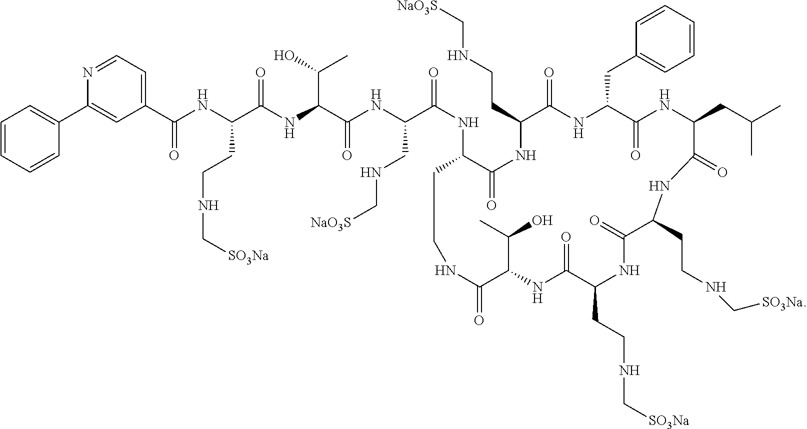

In another embodiment, the present invention provides a compound of Formula IV that is

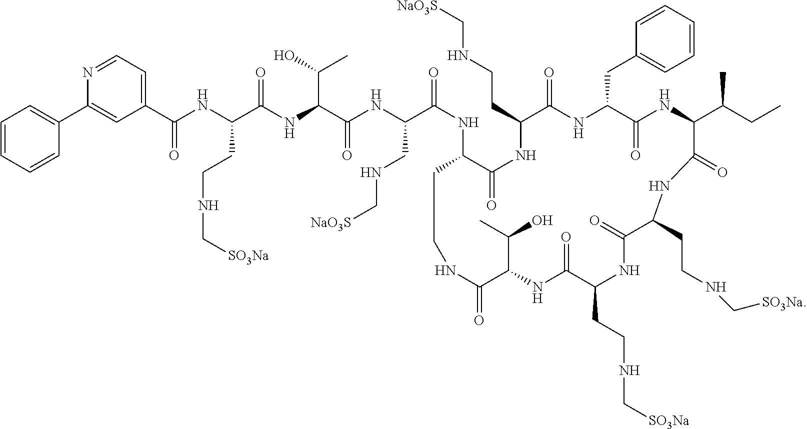

In another embodiment, the present invention provides a compound of Formula IV that is

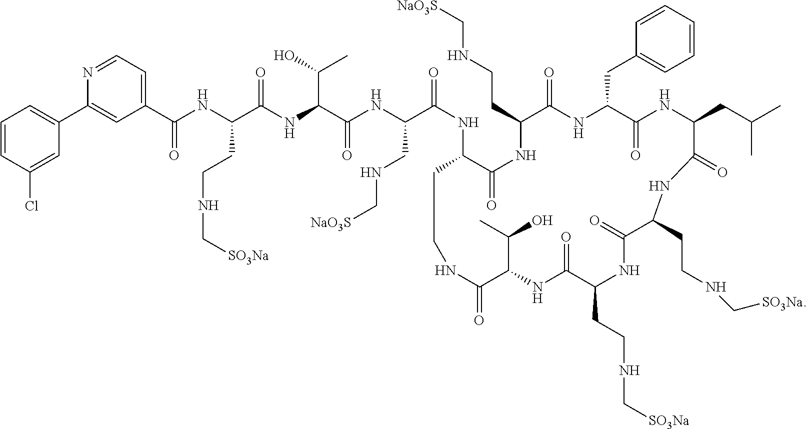

In another embodiment, the present invention provides a compound of Formula IV that is

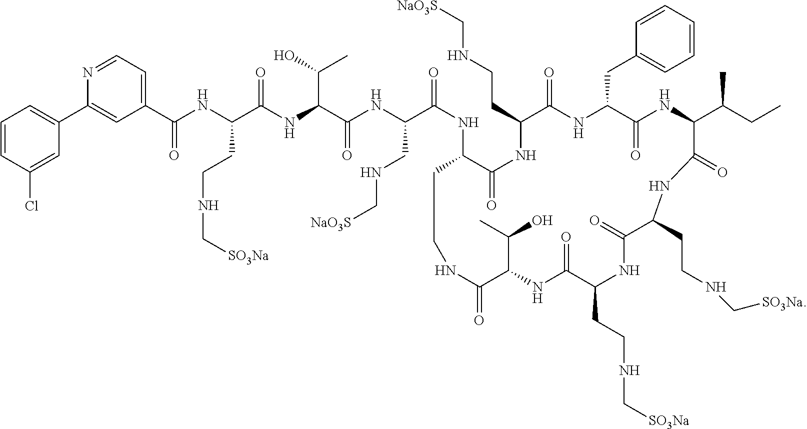

In another embodiment, the present invention provides a compound of Formula IV that is

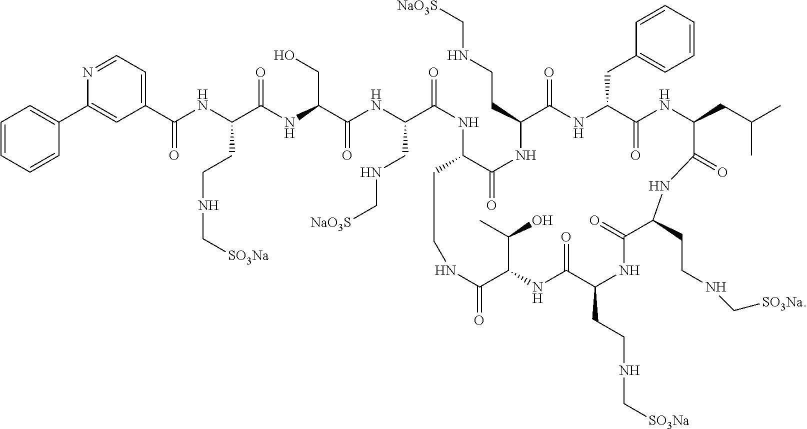

In another embodiment, the present invention provides a compound of Formula IV that is

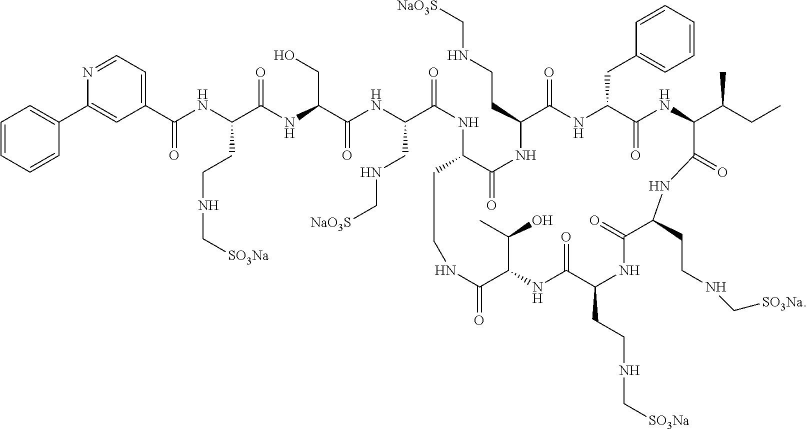

In another embodiment, the present invention provides a compound of Formula IV that is

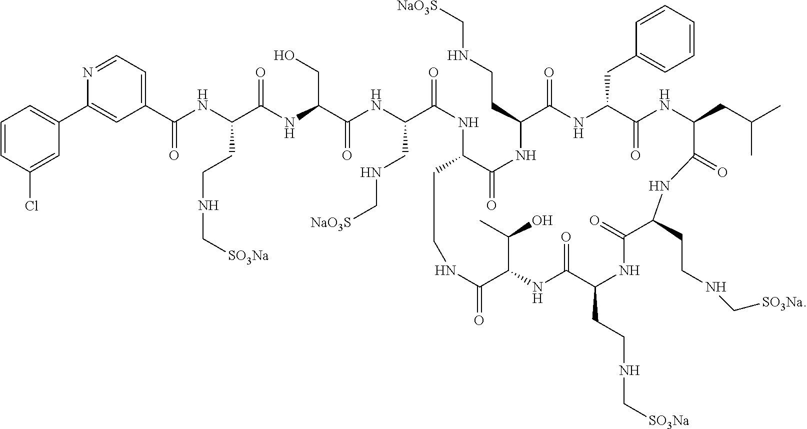

In another embodiment, the present invention provides a compound of Formula IV that is

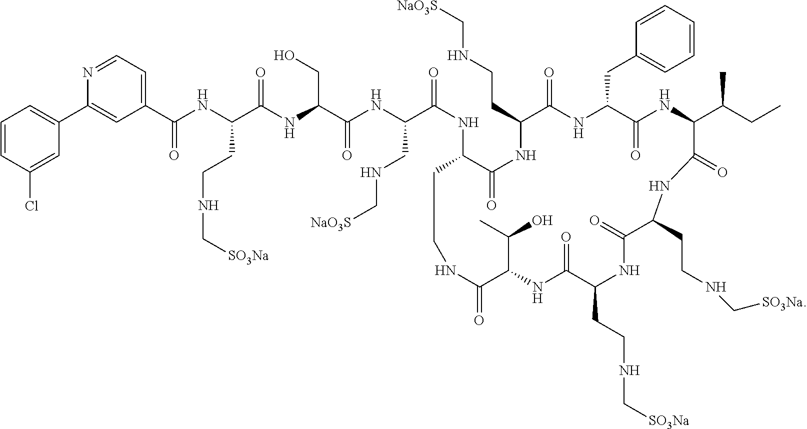

In another embodiment, the present invention provides compounds of Formula IV, or a pharmaceutically acceptable salt thereof, wherein L¹ is —OC(O)—; R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is —OC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; and R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is —OC(O)—; R¹ is (C₅-C₁₂)heteroaryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is —OC(O)—; R¹ is (C₅-C₁₂)heteroaryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is CH₃; R⁶ is H; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; and R⁸ is —CH₂—SO₂OH; n is 0, 1, 2, or 3; and p is 0, 1, 2, or 3.

In another embodiment, the present invention provides compounds of Formula IV, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula IV, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides a pharmaceutical composition comprising a compound of Formula IV, or a pharmaceutically acceptable salt thereof, in admixture with at least one pharmaceutically acceptable excipient.

In another embodiment, the present invention provides a method for treating bacterial infections in a mammal, in particular humans, comprising administering to the mammal in need of such treatment a therapeutically effect amount of a compound of Formula IV, or a pharmaceutically acceptable salt thereof. The methods of the present invention are particularly useful for treating Gram-negative bacterial infections including, but not limited to, infections associated with Pseudomonas aeruginosa, Acinetobacter baumannii, and Klebsiella pneumoniae.

In another embodiment, the present invention provides a method for treating nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, or central nervous system infections comprising administering to the mammal, particularly a human, in need of such treatment a therapeutically effect amount of a compound of Formula IV, or a pharmaceutically acceptable salt thereof.

In another embodiment, the present invention provides the use of a compound of Formula IV, or a pharmaceutically acceptable salt thereof, in the manufacture of a medicament for treating bacterial infections, particularly Gram-negative bacterial infections.

In another embodiment, the present invention provides a compound of Formula V

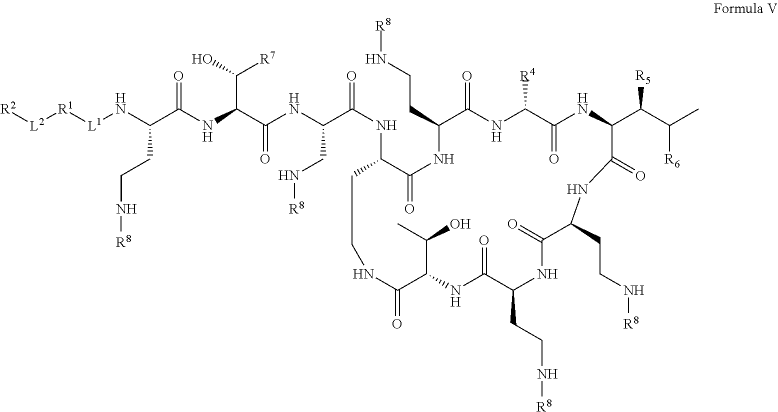

or a pharmaceutically acceptable salt thereof
wherein

L¹ is a bond, carbonyl, sulfonyl, —NHC(O)—, or —OC(O)—;

R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;

L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;

R² is absent, (C₆-C₁₀)aryl, or (C₅-C₁₂)heteroaryl;

R³ is H or (C₁-C₃)alkyl;

R⁴ is (C₁-C₆)alkyl or (C₆-C₁₀)aryl(C₁-C₆)alkyl;

R⁵ is H or CH₃;

R⁶ is H or CH₃;

wherein R⁶ is H when R⁵ is CH₃ and wherein R⁶ is CH₃ when R⁵ is H;

R⁷ is H or CH₃;

R⁸ is (C₁-C₁₀)alkylcarbonyl, (C₁-C₁₀)alkyl-O-carbonyl, (C₆-C₁₀)aryl-O-carbonyl, or —CH₂—SO₂OH;

n is 0, 1, 2, or 3; and

p is 0, 1, 2, or 3.

In one embodiment, the present invention provides compounds of Formula V, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n iso, 1, 2, or 3; and p is 0, 1, 2, or 3.

In another embodiment, the present invention provides compounds of Formula V, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is —CH₂CH(CH₃)₂; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0; and p is 0.

In one embodiment, the present invention provides compounds of Formula V, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is a bond, carbonyl, sulfonyl, or —NHC(O)—; R¹ is (C₁-C₁₀)alkyl, (C₆-C₁₀)aryl, or (C₅-C₁₂)heteroaryl; L² is absent, a bond, or —(CH₂)ₙO(CH₂)ₚ—; R² is absent, (C₆-C₁₀)aryl, or (C₅-C₁₂)heteroaryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0; and p is 0.

In another embodiment, the present invention provides compounds of Formula V, or a pharmaceutically acceptable salt thereof, wherein L¹ is carbonyl; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is carbonyl; R¹ is (C₁-C₁₀)alkyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₁-C₁₀)alkyl; L² is a bond; R² is phenyl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₁-C₁₀)alkyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₁-C₁₀)alkyl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₁-C₁₀)alkyl; L² is a bond; R² is phenyl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is carbonyl; R¹ is (C₆-C₁₀)aryl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is (C₁-C₆)alkyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl; L² is a bond; R² is (C₆-C₁₀)aryl; R⁴ is benzyl; R⁵ is CH₃; R⁶ is H; R⁷ is CH₃; and R⁵ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is (C₆-C₁₀)aryl; R⁴ is benzyl; R⁵ is CH₃; R⁶ is H; R⁷ is CH₃; and R⁵ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is (C₁-C₆)alkoxy, cyano, or halogen; L² is a bond; R² is phenyl optionally substituted with 1 substituent that is (C₁-C₆)alkyl, cyano, or halogen; R⁴ is benzyl; R⁵ is CH₃; R⁶ is H; R⁷ is CH₃; and R⁵ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl; L² is a bond; R² is (C₅-C₁₂)heteroaryl; R⁴ is benzyl; R⁵ is CH₃; R⁶ is H; R⁷ is CH₃; and R⁵ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is halogen; L² is a bond; R² is (C₅-C₁₂)heteroaryl; R⁴ is benzyl; R⁵ is CH₃; R⁶ is H; R⁷ is CH₃; and R⁵ is —CH₂—SO₂OH.

In another embodiment, the present invention provides compounds of Formula V, or a pharmaceutically acceptable salt thereof, wherein L¹ is carbonyl; R¹ is phenyl optionally substituted with 1 substituent that is halogen; L² is a bond; R² is pyridinyl optionally substituted with 1 substituent that is cyano; R⁴ is benzyl; R⁵ is CH₃; R⁷ is CH₃; R⁶ is H; and R⁵ is —CH₂—SO₂OH.

In another embodiment, the present invention provides compounds of Formula V, or a pharmaceutically acceptable salt thereof, wherein L¹ is carbonyl; R¹ is (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH n is 0, 1, 2, or 3; and p is 0, 1, 2, or 3.

In another embodiment, the present invention provides compounds of Formula V, or a pharmaceutically acceptable salt thereof, wherein L¹ is carbonyl; R¹ is (C₅-C₁₂)heteroaryl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₅-C₁₂)heteroaryl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₅-C₁₂)heteroaryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is pyridazinyl or pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is pyridinyl optionally substituted with 1 substituent that is (C₁-C₆)alkoxy; L² is a bond; R² is phenyl optionally substituted with 1 or 2 substituents that are independently (C₁-C₆)alkoxy, cyano, or halogen; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₅-C₁₂)heteroaryl; L² is —(CH₂)ₙO(CH₂)ₚ—; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is carbonyl; R¹ is pyridinyl; L² is —(CH₂)ₙO(CH₂)ₚ—; R² is (C₆-C₁₀)aryl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is CH₃; R⁸ is —CH₂—SO₂OH; n is 0; and p is 0.

In another embodiment, the present invention provides compounds of Formula V, or a pharmaceutically acceptable salt thereof, wherein L¹ is —NHC(O)—; R¹ is (C₆-C₁₀)aryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is —NHC(O)—; R¹ is phenyl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ sulfonyl; R¹ is (C₆-C₁₀)aryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is sulfonyl; R¹ is phenyl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is —NHC(O)—; R¹ is (C₆-C₁₀)aryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is —NHC(O)—; R¹ is phenyl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ sulfonyl; R¹ is (C₆-C₁₀)aryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is sulfonyl; R¹ is phenyl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides a compound of Formula V that is

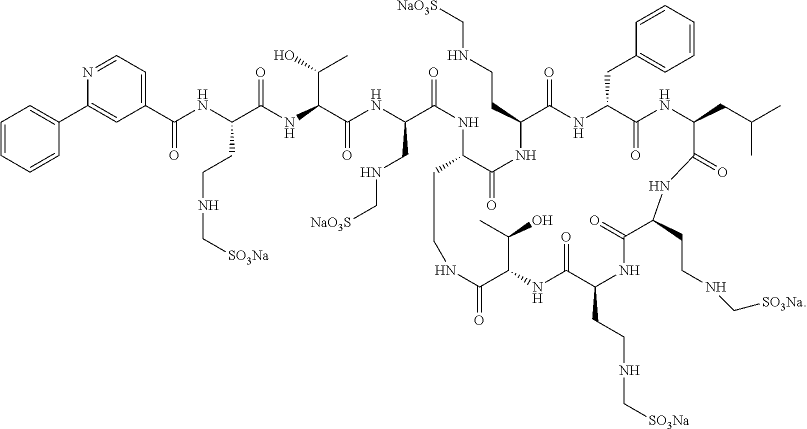

In another embodiment, the present invention provides a compound of Formula V that is

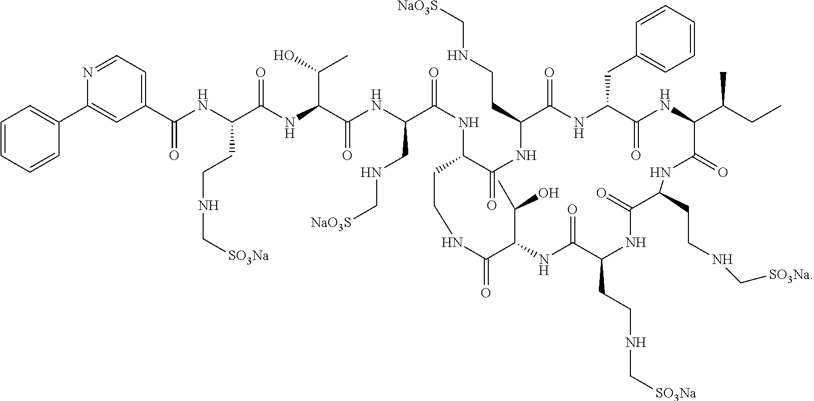

In another embodiment, the present invention provides a compound of Formula V that is

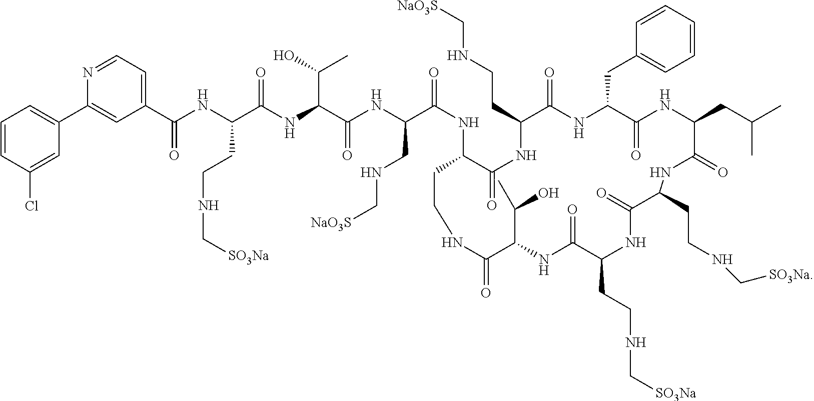

In another embodiment, the present invention provides a compound of Formula V that is

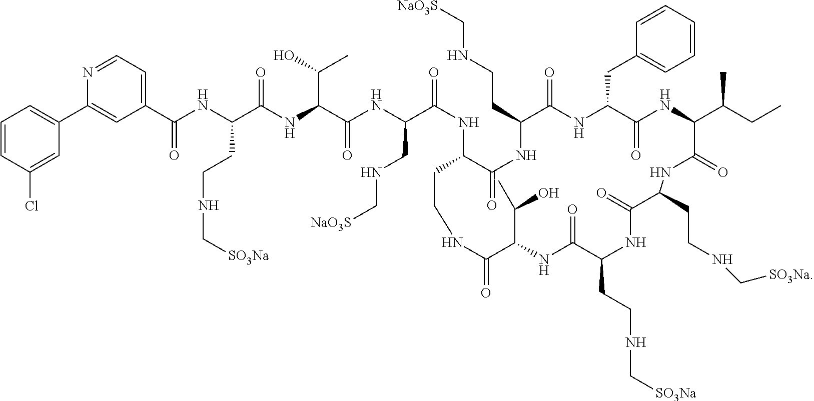

In another embodiment, the present invention provides a compound of Formula V that is

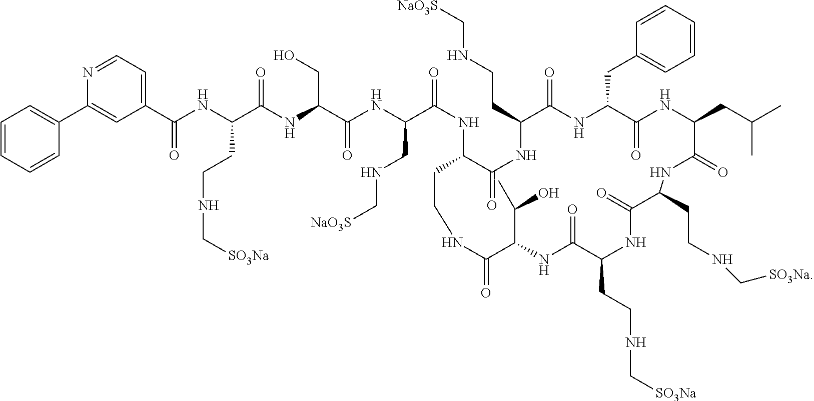

In another embodiment, the present invention provides a compound of Formula V that is

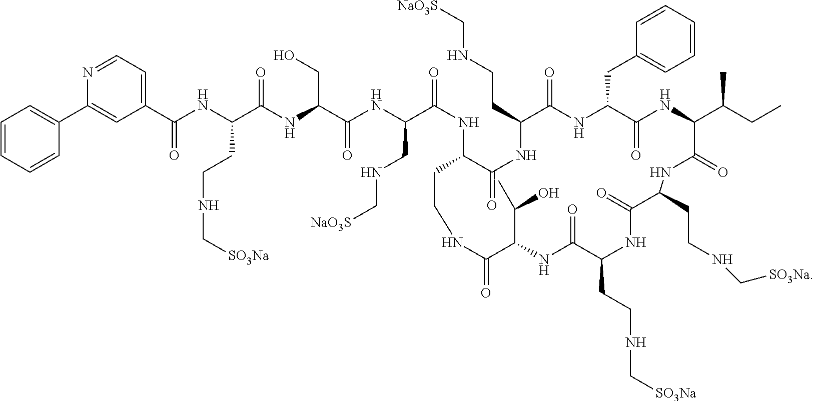

In another embodiment, the present invention provides a compound of Formula V that is

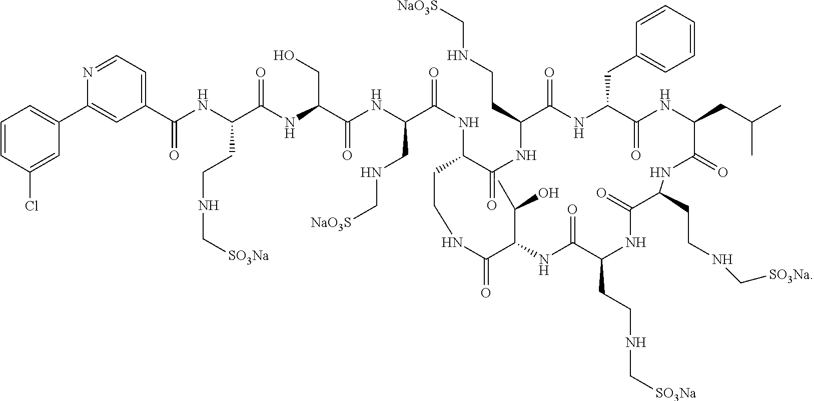

In another embodiment, the present invention provides a compound of Formula V that is

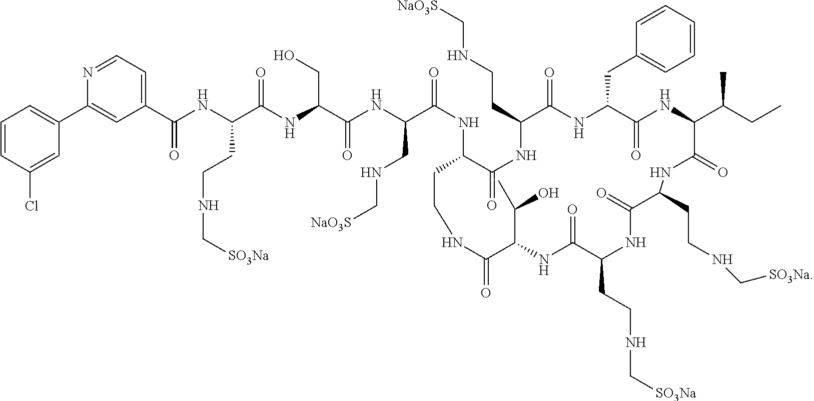

In another embodiment, the present invention provides compounds of Formula V, or a pharmaceutically acceptable salt thereof, wherein L¹ is —OC(O)—; R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is —OC(O)—; R¹ is (C₁-C₁₀)alkyl, (C₆-C₁₀)aryl, or (C₅-C₁₂)heteroaryl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is (C₁-C₆)alkyl or (C₆-C₁₀)aryl(C₁-C₆)alkyl; R⁵ is H or CH₃; R⁶ is H or CH₃, wherein R⁶ is H when R⁵ is CH₃ and wherein R⁶ is CH₃ when R⁵ is H; R⁷ is H or CH₃; and R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is —OC(O)—; R¹ is (C₁-C₁₀)alkyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is —OC(O)—; R¹ is (C₅-C₁₂)heteroaryl; L² is a bond; R² is (C₆-C₁₀)aryl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is —OC(O)—; R¹ is (C₁-C₁₀)alkyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is —OC(O)—; R¹ is (C₅-C₁₂)heteroaryl; L² is a bond; R² is (C₆-C₁₀)aryl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is —OC(O)—; R¹ is pyridinyl; L² is a bond; R² is phenyl optionally substituted with Cl; R⁴ is benzyl; R⁵ is CH₃; R⁶ is H; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl(C₂-C₄)alkenyl; L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—; R² is absent, (C₆-C₁₀)aryl, or (C₅-C₁₂)heteroaryl; R³ is H or (C₁-C₃)alkyl; R⁴ is benzyl; R⁵ is H or CH₃; R⁶ is H or CH₃, wherein R⁶ is H when R⁵ is CH₃ and wherein R⁶ is CH₃ when R⁵ is H; R⁷ is H or CH₃; and R⁸ is —CH₂—SO₂OH; n is 0, 1, 2, or 3; and p is 0, 1, 2, or 3.

In another embodiment, the present invention provides compounds of Formula V, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is H; R⁶ is CH₃; R⁷ is CH₃; and R⁸ is —CH₂—SO₂OH.

In another embodiment, the present invention provides compounds of Formula V, or a pharmaceutically acceptable salt thereof, wherein L¹ is carbonyl; R¹ is (C₆-C₁₀)aryl(C₂-C₄)alkenyl; L² is absent; R² is absent; R⁴ is benzyl; R⁵ is CH₃; R⁶ is H; R⁷ is CH₃; and R⁸ is —CH₂—SO₂OH.

In another embodiment, the present invention provides a pharmaceutical composition comprising a compound of Formula V, or a pharmaceutically acceptable salt thereof, in admixture with at least one pharmaceutically acceptable excipient.

In another embodiment, the present invention provides a method for treating bacterial infections in a mammal, in particular humans, comprising administering to the mammal in need of such treatment a therapeutically effect amount of a compound of Formula V, or a pharmaceutically acceptable salt thereof. The methods of the present invention are particularly useful for treating Gram-negative bacterial infections including, but not limited to, infections associated with Pseudomonas aeruginosa, Acinetobacter baumannii, and Klebsiella pneumoniae.

In another embodiment, the present invention provides a method for treating nosocomial pneumonia, urinary tract infections, systemic infections (bacteremia and sepsis), skin and soft tissue infections, surgical infections, intraabdominal infections, lung infections (including those in patients with cystic fibrosis), Helicobacter pylori (and relief of associated gastric complications such as peptic ulcer disease, gastric carcinogenesis, etc.), endocarditis, diabetic foot infections, osteomyelitis, or central nervous system infections comprising administering to the mammal, particularly a human, in need of such treatment a therapeutically effect amount of a compound of Formula V, or a pharmaceutically acceptable salt thereof.

In another embodiment, the present invention provides the use of a compound of Formula V, or a pharmaceutically acceptable salt thereof, in the manufacture of a medicament for treating bacterial infections, particularly Gram-negative bacterial infections.

### DEFINITIONS

As used throughout this specification and the appended claims, the following terms have the following meanings:

The term “(C₂-C₄)alkenyl” as used herein, means a straight or branched chain hydrocarbon containing from 2 to 4 carbons and containing at least one carbon-carbon double bond. Representative examples of (C₂-C₄)alkenyl include, but are not limited to, ethenyl, 2-propenyl, 2-methyl-2-propenyl, and 3-butenyl.

The term “(C₂-C₆)alkenyl” as used herein, means a straight or branched chain hydrocarbon containing from 2 to 6 carbons and containing at least one carbon-carbon double bond. Representative examples of (C₂-C₆)alkenyl include, but are not limited to, ethenyl, 2-propenyl, 2-methyl-2-propenyl, 3-butenyl, 4-pentenyl, and 5-hexenyl.

The term “(C₁-C₆)alkoxy” as used herein, means a (C₁-C₆)alkyl group, as defined herein, appended to the parent molecular moiety through an oxygen atom. Representative examples of (C₁-C₆)alkoxy include, but are not limited to, methoxy, ethoxy, propoxy, 2-propoxy, butoxy, tert-butoxy, pentyloxy, and hexyloxy.

The term “(C₁-C₆)alkoxy(C₁-C₆)alkyl” as used herein, means a (C₁-C₆)alkoxy group, as defined herein, appended to the parent molecular moiety through a (C₁-C₆)alkyl group, as defined herein. Representative examples of (C₁-C₆)alkoxy(C₁-C₆)alkyl include, but are not limited to, tert-butoxymethyl, 2-ethoxyethyl, 2-methoxyethyl, and methoxymethyl.

The term “(C₁-C₆)alkoxycarbonyl” as used herein, means a (C₁-C₆)alkoxy group, as defined herein, appended to the parent molecular moiety through a carbonyl group, as defined herein. Representative examples of (C₁-C₆)alkoxycarbonyl include, but are not limited to, methoxycarbonyl, ethoxycarbonyl, and tert-butoxycarbonyl.

The term “(C₁-C₆)alkoxysulfonyl” as used herein, means a (C₁-C₆)alkoxy group, as defined herein, appended to the parent molecular moiety through a sulfonyl group, as defined herein. Representative examples of (C₁-C₆)alkoxysulfonyl include, but are not limited to, methoxysulfonyl, ethoxysulfonyl and propoxysulfonyl.

The term “(C₁-C₃)alkyl” as used herein, means a straight or branched chain hydrocarbon containing from 1 to 3 carbon atoms. Examples of (C₁-C₃)alkyl include methyl, ethyl, n-propyl, and iso-propyl.

The term “(C₁-C₆)alkyl” as used herein, means a straight or branched chain hydrocarbon containing from 1 to 6 carbon atoms. Representative examples of (C₁-C₆)alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “(C₁-C₁₀)alkyl” as used herein, means a straight or branched chain hydrocarbon containing from 1 to 6 carbon atoms. Representative examples of (C₁-C₁₀)alkyl include, but are not limited to, methyl, ethyl, n-propyl, iso-propyl, n-butyl, sec-butyl, iso-butyl, tert-butyl, n-pentyl, isopentyl, neopentyl, n-hexyl, n-heptyl, n-octyl, n-nonyl, n-decyl, 5-methylheptyl, and 5-methylhexyl.

The term “(C₁-C₆)alkylcarbonyl” as used herein, means a (C₁-C₆)alkyl group, as defined herein, appended to the parent molecular moiety through a carbonyl group, as defined herein. Representative examples of (C₁-C₆)alkylcarbonyl include, but are not limited to, acetyl, 1-oxopropyl, 2,2-dimethyl-1-oxopropyl, 1-oxobutyl, and 1-oxopentyl.

The term “(C₁-C₆)alkylcarbonyloxy” as used herein, means a (C₁-C₆)alkylcarbonyl group, as defined herein, appended to the parent molecular moiety through an oxygen atom. Representative examples of (C₁-C₆)alkylcarbonyloxy include, but are not limited to, acetyloxy, ethylcarbonyloxy, and tert-butylcarbonyloxy.

The term “(C₁-C₆)alkylsulfinyl” as used herein, means an (C₁-C₆)alkyl group, as defined herein, appended to the parent molecular moiety through a sulfinyl group, as defined herein. Representative examples of (C₁-C₆)alkylsulfinyl include, but are not limited to, methylsulfinyl and ethylsulfinyl.

The term “(C₁-C₆)alkylsulfonyl” as used herein, means an (C₁-C₆)alkyl group, as defined herein, appended to the parent molecular moiety through a sulfonyl group, as defined herein. Representative examples of (C₁-C₆)alkylsulfonyl include, but are not limited to, methylsulfonyl and ethylsulfonyl.

The term “(C₁-C₆)alkylthio” as used herein, means a (C₁-C₆)alkyl group, as defined herein, appended to the parent molecular moiety through a sulfur atom. Representative examples of (C₁-C₆)alkylthio include, but are not limited to, methylthio, ethylthio, tert-butylthio, and hexylthio.

The term “(C₁-C₆)alkylthio(C₁-C₆)alkyl” as used herein, means a (C₁-C₆)alkylthio group, as defined herein, appended to the parent molecular moiety through a (C₁-C₆)alkyl group, as defined herein. Representative examples of (C₁-C₆)alkylthio(C₁-C₆)alkyl include, but are not limited to, methylthiomethyl and 2-(ethylthio)ethyl.

The term “(C₂-C₆)alkynyl” as used herein, means a straight or branched chain hydrocarbon group containing from 2 to 6 carbon atoms and containing at least one carbon-carbon triple bond. Representative examples of (C₂-C₆)alkynyl include, but are not limited to, to acetylenyl, 1-propynyl, 2-propynyl, 3-butynyl, 2-pentynyl, and 1-butynyl.

The term “(C₆-C₁₀)aryl” as used herein, means phenyl or naphthyl.

The (C₆-C₁₀)aryl groups of the invention are optionally substituted with 1, 2, 3, 4, or 5 groups that are independently (C₂-C₆)alkenyl, (C₁-C₆)alkoxy, (C₁-C₆)alkoxy(C₁-C₆)alkyl, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfinyl, (C₁-C₆)alkylsulfonyl, (C₁-C₆)alkylthio, (C₁-C₆)alkylthio(C₁-C₆)alkyl, (C₂-C₆)alkynyl, carboxy, carboxy(C₁-C₆)alkyl, cyano, formyl, halo(C₁-C₆)alkoxy, halo(C₁-C₆)alkyl, halogen, hydroxy, hydroxy(C₁-C₆)alkyl, mercapto, nitro, —NZ¹Z², (NZ¹Z²)carbonyl, (NZ¹Z²)carbonyloxy, or (NZ¹Z²)sulfonyl. Representative examples of substituted aryl include, but are not limited to, 2-chlorophenyl, 3-chlorophenyl, 4-chlorophenyl, 2-cyanophenyl, 3-cyanophenyl, 4-cyanophenyl, 4-cyano-3-fluorophenyl, 2-fluorophenyl, 3-fluorophenyl, 4-fluorophenyl, 3-isopropylphenyl, 2-methoxyphenyl, 3-methoxyphenyl, 4-methoxyphenyl, 2-methylphenyl, 3-methylphenyl, and 4-methylphenyl.

The term “(C₆-C₁₀)aryl(C₂-C₄)alkenyl” as used herein, means a (C₆-C₁₀)aryl group, as defined herein, appended to the parent molecular moiety through a (C₂-C₄)alkenyl group, as defined herein. Representative examples of (C₆-C₁₀)aryl(C₂-C₄)alkenyl include, but are not limited to, styrene, 3-phenylprop-1-en-yl, 4-phenylbut-1-en-1-yl, and 4-phenylbut-3-en-1-yl.

The term “(C₆-C₁₀)aryl(C₁-C₆)alkyl” as used herein, means a (C₆-C₁₀)aryl group, as defined herein, appended to the parent molecular moiety through a (C₁-C₆)alkyl group, as defined herein. Representative examples of (C₆-C₁₀)aryl(C₁-C₆)alkyl include, but are not limited to, benzyl, 2-phenylethyl, and 4-phenylbutyl.

The term “(C₆-C₁₀)arylcarbonyl” as used herein, means a (C₆-C₁₀)aryl group, as defined herein, appended to the parent molecular moiety through a carbonyl group, as defined herein. Representative examples of (C₆-C₁₀)arylcarbonyl include, but are not limited to, acetyl, 1-oxopropyl, 2,2-dimethyl-1-oxopropyl, 1-oxobutyl, and 1-oxopentyl.

The term “benzyl” as used herein, means a —CH₂-Phenyl group.

The term “carbonyl” as used herein, means a —C(O)— group.

The term “carboxy” as used herein, means a —CO₂H group.

The term “carboxy(C₁-C₆)alkyl” as used herein, means a carboxy group, as defined herein, is attached to the parent molecular moiety through a (C₁-C₆)alkyl group.

The term “cyano” as used herein, means a —CN group.

The term “formyl” as used herein, means a —C(O)H group.

The term “halo” or “halogen” as used herein, means —Cl, —Br, —I or —F.

The term “halo(C₁-C₃)alkoxy” as used herein, means at least one halogen, as defined herein, appended to the parent molecular moiety through a (C₁-C₃)alkoxy group, as defined herein. Representative examples of halo(C₁-C₃)alkoxy include, but are not limited to, chloromethoxy, 2-fluoroethoxy, trifluoromethoxy, and pentafluoroethoxy.

The term “halo(C₁-C₆)alkyl” as used herein, means at least one halogen, as defined herein, appended to the parent molecular moiety through a (C₁-C₆)alkyl group, as defined herein. Representative examples of halo(C₁-C₆)alkyl include, but are not limited to, chloromethyl, 2-fluoroethyl, trifluoromethyl, pentafluoroethyl, and 2-chloro-3-fluoropentyl.

The term “(C₅-C₁₂)heteroaryl” as used herein, means a monocyclic heteroaryl or a bicyclic heteroaryl. The monocyclic heteroaryl is a 5 or 6 membered ring. The 5 membered ring consists of two double bonds and one, two, three or four nitrogen atoms and/or optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothienyl, benzoxadiazolyl, benzoxazolyl, benzothiazolyl, furopyridinyl, indolyl, isoquinolinyl, naphthyridinyl, phthalazinyl, pyrrolopyridinyl, quinazolinyl, quinolinyl, quinoxalinyl, and thienopyridinyl.

The (C₅-C₁₂)heteroaryl groups of the invention are optionally substituted with 1, 2, 3, or 4 groups that are independently (C₂-C₆)alkenyl, (C₁-C₆)alkoxy, (C₁-C₆)alkoxy(C₁-C₆)alkyl, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfinyl, (C₁-C₆)alkylsulfonyl, (C₁-C₆)alkylthio, (C₁-C₆)alkylthio(C₁-C₆)alkyl, (C₂-C₆)alkynyl, carboxy, carboxy(C₁-C₆)alkyl, cyano, formyl, halo(C₁-C₆)alkoxy, halo(C₁-C₆)alkyl, halogen, hydroxy, hydroxy(C₁-C₆)alkyl, mercapto, nitro, —NZ¹Z², (NZ¹Z²)carbonyl, (NZ¹Z²)carbonyloxy, or (NZ¹Z²)sulfonyl. Heteroaryl groups of the invention that are substituted may be as tautomers. The present invention encompasses all tautomers including non-aromatic tautomers.

The term “(C₅-C₁₂)heteroaryl(C₂-C₄)alkenyl” as used herein means, a (C₅-C₁₂)heteroaryl group, as defined herein, appended to the parent molecular moiety through a (C₂-C₄)alkenyl group, as defined herein. Representative examples of (C₅-C₁₂)heteroaryl(C₂-C₄)alkenyl include, but are not limited to, (E)-2-(pyridin-4-yl)vinyl, (E)-2-(pyridin-2-yl)vinyl, and (E)-2-(pyridin-2-yl)vinyl.

The term “hydroxy” as used herein, means an —OH group.

The term “hydroxy(C₁-C₆)alkyl” as used herein, means at least one hydroxy group, as defined herein, is appended to the parent molecular moiety through a (C₁-C₆)alkyl group, as defined herein.

The term “mercapto” as used herein, means a —SH group.

The term “nitro” as used herein, means a —NO₂ group.

The term “NZ¹Z²” as used herein, means two groups, Z¹ and Z², which are appended to the parent molecular moiety through a nitrogen atom. Z¹ and Z² are each independently H, (C₁-C₆)alkyl, (C₁-C₆)alkylcarbonyl, or formyl. Representative examples of NZ¹Z² include, but are not limited to, amino, methylamino, acetylamino, acetylmethylamino, butylamino, diethylamino, dimethylamino, ethylmethylamino, and formylamino.

The term “(NZ¹Z²)carbonyl” as used herein, means a NZ¹Z² group, as defined herein, appended to the parent molecular moiety through a carbonyl group, as defined herein. Representative examples of (NZ¹Z²)carbonyl include, but are not limited to, aminocarbonyl, (methylamino)carbonyl, (dimethylamino)carbonyl, and (ethylmethylamino)carbonyl.

The term “(NZ¹Z²)carbonyloxy” as used herein, means a (NZ¹Z²)carbonyl group, as defined herein, appended to the parent molecular moiety through an oxygen atom.

The term “(NZ¹Z²)sulfonyl” as used herein, means a NZ¹Z² group, as defined herein, appended to the parent molecular moiety through a sulfonyl group, as defined herein. Representative examples of (NZ¹Z²)sulfonyl include, but are not limited to, aminosulfonyl, (methylamino)sulfonyl, (dimethylamino)sulfonyl, and (ethylmethylamino)sulfonyl.

The term “sulfinyl” as used herein, means a —S(O)— group.

The term “sulfonyl” as used herein, means a —SO₂— group.

The phrase “therapeutically effective amount” means an amount of a compound of Formula I or Formula II that, when administered to a patient, provides the desired effect; i.e., lessening in the severity of the symptoms associated with a bacterial infection, decreasing the number of bacteria in the affected tissue, and/or preventing bacteria in the affected tissue from increasing in number (localized or systemic).

The term “patient” means a warm blooded animals such as for example, livestock, guinea pigs, mice, rats, gerbils, cats, rabbits, dogs, monkeys, chimpanzees, and humans.

The term “treat” means the ability of the compounds to relieve, alleviate or slow the progression of the patient's bacterial infection (or condition) or any tissue damage associated with the disease.

The phrase “pharmaceutically acceptable” indicates that the substance or composition must be compatible chemically and/or toxicologically, with the other ingredients comprising a formulation, and/or the mammal being treated therewith.

The term “isomer” means “stereoisomer” and “geometric isomer” as defined below.

The term “stereoisomer” means compounds that possess one or more chiral centers and each center may exist in the (R) or (S) configuration. Stereoisomers include all diastereomeric, enantiomeric and epimeric forms as well as racemates and mixtures thereof.

The term “geometric isomer” means compounds that may exist in cis, trans, anti, entgegen (E), and zusammen (Z) forms as well as mixtures thereof.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above. Preferred salts of the present invention are trifluoroacetate (CF₃CO₂H), tosylate (CH₃C₆H₄SO₂OH), sulfate (H₂SO₄), and hydrochloride (HCl).

The invention also relates to base addition salts of the compounds of the invention. The chemical bases that may be used as reagents to prepare these pharmaceutically acceptable base salts are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Suitable base salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art.

Certain of the compounds of the formula (I) may exist as geometric isomers. The compounds of the formula (I) may possess one or more asymmetric centers, thus existing as two or more stereoisomeric forms. The present invention includes all the individual stereoisomers and geometric isomers of the compounds of formula (I) and mixtures thereof. Individual enantiomers can be obtained by chiral separation or using the relevant enantiomer in the synthesis.

In addition, the compounds of the present invention can exist in unsolvated as well as solvated forms with pharmaceutically acceptable solvents such as water, ethanol and the like. In general, the solvated forms are considered equivalent to the unsolvated forms for the purposes of the present invention. The compounds may also exist in one or more crystalline states, i.e. polymorphs, or they may exist as amorphous solids. All such forms are encompassed by the claims.

The invention also relates to prodrugs of the compounds of the invention. Thus certain derivatives of compounds of the invention which may have little or no pharmacological activity themselves can, when administered into or onto the body, be converted into compounds of the invention having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

This invention also encompasses compounds of the invention containing protective groups. One skilled in the art will also appreciate that compounds of the invention can also be prepared with certain protecting groups that are useful for purification or storage and can be removed before administration to a patient. The protection and deprotection of functional groups is described in “Protective Groups in Organic Chemistry”, edited by J. W. F. McOmie, Plenum Press (1973) and “Protective Groups in Organic Synthesis”, 3rd edition, T. W. Greene and P. G. M. Wuts, Wiley-Interscience (1999).

The present invention also includes isotopically-labeled compounds, which are identical to those recited in Formula I or Formula II, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as, but not limited to, ²H, ³H, ¹³C, ¹⁴C, ¹⁵N, ¹⁷O, ¹⁸O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of this invention and prodrugs thereof can generally be prepared by carrying out the procedures disclosed in the Schemes and/or in the Examples below, by substituting a readily available isotopically-labeled reagent for a non-isotopically-labeled reagent.

### Medical and Veterinary Uses

The compounds may be used for the treatment or prevention of infectious disorders, especially those caused by susceptible and multi-drug resistant (MDR) Gram-negative bacteria. Examples of such Gram-negative bacteria include Acinetobacter baumannii, Acinetobacter spp., Achromobacter spp., Aeromonas spp., Bacteroides fragilis, Bordetella spp., Borrelia spp., Brucella spp., Campylobacter spp., Citrobacter diversus (koseri), Citrobacter freundii, Enterobacter aerogenes, Enterobacter cloacae, Escherichia coli, Francisella tularensis, Fusobacterium spp., Haemophilus influenzae (β-lactamase positive and negative), Helicobacter pylori, Klebsiella oxytoca, Klebsiella pneumoniae (including those encoding extended-spectrum β-lactamases (hereinafter “ESBLs”), Legionella pneumophila, Moraxella catarrhalis (β-lactamase positive and negative), Morganella morganii, Neisseria gonorrhoeae, Neisseria meningitidis, Proteus vulgaris, Porphyromonas spp., Prevotella spp., Mannheimia haemolyticus, Pasteurella spp., Proteus mirabilis, Providencia spp., Pseudomonas aeruginosa, Pseudomonas spp., Salmonella spp., Shigella spp., Serratia marcescens, Treponema spp., Burkholderia cepacia, Vibrio spp., Yersinia spp., and Stenotrophomonas mulophilia. Examples of other gram negative organisms include members of the Enterobacteriaceae that express ESBLs; KPCs, CTX-M, metallo-β-lactamases (such as NDM-1, for exmple), and AmpC-type beta-lactamases that confer resistance to currently available cephalosporins, cephamycins, carbapenems, and beta-lactam/beta-lactamase inhibitor combinations.

In a more specific embodiment, the Gram-negative bacteria are selected from the group consisting of Acinetobacter baumannii, Acinetobacter spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia, Pseudomonas aeruginosa and members of the Enterobacteriaceae and Pseudomonas that express ESBLs, KPCs, CTX-M, metallo-β-lactamases, and AmpC-type beta-lactamases that confer resistance to currently available cephalosporins, cephamycins, carbapenems, and beta-lactam/beta-lactamase inhibitor combinations.

Examples of infections that may be treated with the compounds of Formula I or Formula II include nosocomial pneumonia, urinary tract infections, systemic infections (bacteremia and sepsis), skin and soft tissue infections, surgical infections, intraabdominal infections, lung infections in patients with cystic fibrosis, patients suffering from lung infections, endocarditis, diabetic foot infections, osteomyelitis, and central nervous system infections.

In addition, the compounds can be used to treat Helicobacter pylori infections in the GI tract of humans (and other mammals). Elimination of these bacteria is associated with improved health outcomes including fewer dyspeptic symptoms, reduced peptic ulcer recurrence and rebleeding, reduced risk of gastric cancer, etc. A more detailed discussion of eradicating H. pylori and its impact on gastrointestinal illness may be found at: www.informahealthcare.com, Expert Opin. Drug Saf. (2008) 7(3).

In order to exhibit this anti-infective activity, the compounds of Formula I or Formula II need to be administered in a therapeutically effective amount. A “therapeutically effective amount” is meant to describe a sufficient quantity of the compound to treat the infection, at a reasonable benefit/risk ratio applicable to any such medical treatment. It will be understood, however, that the attending physician, within the scope of sound medical judgment, will decide the total daily dosage of the compound. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. As a general guideline however, the total daily dose will typically range from about 0.1 mg/kg/day to about 5000 mg/kg/day in single or in divided doses. Typically, dosages for humans will range from about 10 mg to about 3000 mg per day, in a single or multiple doses.

Any route typically used to treat infectious illnesses, including oral, parenteral, topical, rectal, transmucosal, and intestinal, can be used to administer the compounds. Parenteral administrations include injections to generate a systemic effect or injections directly into to the afflicted area. Examples of parenteral administrations are subcutaneous, intravenous, intramuscular, intradermal, intrathecal, and intraocular, intranasal, intravetricular injections or infusions techniques. Topical administrations include the treatment of areas readily accessibly by local application, such as, for example, eyes, ears including external and middle ear infections, vaginal, open wound, skin including the surface skin and the underneath dermal structures, or lower intestinal tract. Transmucosal administration includes nasal aerosol or inhalation applications.

### Formulations

Compounds of the invention can be formulated for administration in any way for use in human or veterinary medicine, by analogy with other bioactive agents such as antibiotics. Such methods are known in the art and are summarized below.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, ophthalmic ointments/drops and otic drops, impregnated dressings and aerosols, and may contain appropriate conventional additives such as preservatives, solvents to assist drug penetration and emollients, etc. Such topical formulations may also contain conventional carriers, such as cream or ointment bases and ethanol or oleyl alcohol for lotions. Such carriers may be present, for example, from about 1% up to about 98% of the formulation.

Tablets and capsules for oral administration may be in unit dose presentation form, and may contain conventional excipients such as binding agents, for example acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being typical. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, to about 100% by weight, of the active material, depending on the method of administration. Where the compositions comprise dosage units, each unit will contain, for example, from about 0.5-1000 mg of the active ingredient. The dosage as employed for adult human treatment will range, for example, from about 10 to 3000 mg per day, depending on the route and frequency of administration.

If desired, the compounds of the invention may be administered in combination with one or more additional anti-bacterial agents (“the additional active agent”). Such use of compounds of the invention in combination with an additional active agent may be for simultaneous, separate or sequential use.

Compounds of the present invention, described by Formula I as either an individual compound or as its leucine/isoleucine combination, may be administered in the form of liposomes that are generally derived from phospholipids or other lipid substances. Any non-toxic, physiologically acceptable and metabolizable lipid capable of forming liposomes may be used. Liposomes of the compounds of the present invention may be prepared using similar procedures as described in Alipour et al., International Journal of Pharmaceutics, 355, 293-298 (2008), incorporated by reference. For example, a compound of Formula I, or its leucine/isoleucine combination, may be combined in a lipid mixture (90 micromoles) of either DPPC or POPC and Chol in a molar ratio of 2:1 using similar methods as described in Omri, A., et al., Biochem. Pharmacol. 64, 1407-1413, incorporated by reference. The chloroform used to dissolve the lipids is removed under vacuum at 51° C. using a rotary evaporator. To the thin dry lipid film, 6 ml of an aqueous solution of the compound of Formula I (or its leucine/isoleucine combination) at a concentration of 10 mg/mL is added. The lipid suspensions, are submerged in an ice-bath and either sonicated for 5 min or extruded twice through a double-stacked 100 nm pore-size polycarbonate membranes (Nucleopore Corp. Pleasanton, Calif., USA) in an extruder (Lipex Biomembranes Inc., Vancouver, BC, Canada). Sonicated samples are freeze-dried overnight for preservation (Labconco model 77540, USA). Dehydrated liposomes are rehydrated in saline above the phase transition temperature of lipids for 2 h and unencapsulated drug is washed off twice by centrifugation. The size of sonicated or extruded liposomal suspensions is determined with the use of a coulter N4SD particle-size analyzer (Coulter Electronics of Canada, Burlington, ON, Canada) and may have a mean diameter of 343±28 nm for sonicated liposomes and 172.5±22 nm for extruded liposomes.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Synthetic Methodology

### Abbreviations:

AA: amino acid
ACN: acetonitrile
Chol: cholesterol
CTC: 2-chlorotrityl chloride
Dab: (S)-2,4-diaminobutanoic acid
Dap: (S)-2,3-diaminopropanoic acid
D-Dap: (R)-2,3-diaminopropionic acid
D-Dap-3: D-Dap as the 3^{rd} amino acid residue of the polymyxin skeleton
Boc: tert-butoxycarbonyl
tBu: tert-butyl
Dde: 1-(4,4-dimethyl-2,6-dioxo-cyclohexylidene)-ethyl
Fmoc: 9H-fluoren-9-ylmethoxycarbonyl
Fmoc-Dab(Boc)-OH: (S)-4-Boc-amino-2-Fmoc-amino-butyric acid
Fmoc-Dab(Dde)-OH: (S)-4-Dde-amino-2-Fmoc-amino-butyric acid
Fmoc-Dap(Boc)-OH: (S)-3-Boc-amino-2-Fmoc-amino-propionic acid

### Phe-OH: L-phenylalanine

### Fmoc-Phe-OH: Fmoc-L-phenylalanine

### DPhe-OH: D-phenylalanine

### Fmoc-D-Phe-OH: Fmoc-D-phenylalanine

### Leu-OH: L-leucine

### Ile: L-isoleucine

Ile-7: Ile as the 7^{th} amino acid residue of the polymyxin skeleton

### Fmoc-Leu-OH: Fmoc-L-leucine

### Thr-OH: L-threonine

### Fmoc-Thr(tBu)-OH: N-Fmoc-O-tert-Butyl-L-threonine

### Ser: L-serine

Ser-2: Ser as the 2^{nd} amino acid residue of the polymyxin skeleton
DCC: N,N′-dicyclohexylcarbodiimide
DCM: dichloromethane
DIC: diisopropyl carbodiimide
DIPEA: diisopropylethylamine
DMF: dimethyl formamide
DMSO: dimethyl sulfoxide
DPPC: 1,2-dipalmitoyl-sn-glycero-3-phosphocholine
EDC-HCl: 1-ethyl-3-(3-dimethylaminopropyl)carbodiimide hydrochloride
HATU: 2-(7-aza-1H-benzotriazole-1-yl)-1,1,3,3-tetramethyluronium hexafluorophosphate

### HOBt: N-hydroxybenzotriazole

LCMS: liquid chromatography mass spectrometry
MeOH: methyl alcohol
NMM: N-methyl morpholine

### NMP: N-methylpyrrolidinone

PG: protecting group
POPC: 1-palmitoyl-2-oleoyl-sn-glycero-3-phosphocholine
Solution F: 95% TFA, 2.5% water, 2.5% triisopropyl silane
t-BME: tert-butyl methyl ether
TEA: triethylamine
TFA: trifluoroacetic acid
RT: room or ambient temperature Z: benzyloxycarbonyl

### ZOSuc: N-(Benzyloxycarbonyloxy)succinimide

### Analytical instrumentation

MS and LCMS: Hewlett-Packard HP1100 or HP1090, Agilent, LCQ Advantage

NMR: Bruker 500 MHz

Optical rotation: Perkin-Elmer (PE) Polarimeter 341

HRMS: All data were collected on an Agilent (Wilmington, Del.) 6220 Accurate Mass TOF LC/MS operating in the electrospray ionization mode. The chromatography system consisted of an Agilent (Wilmington, Del.) 1200 binary pumping system with the addition of an extra isocratic system and a dynamic splitter for dilution of samples as they passed between the UV detector and the mass spectrometer. The binary pump operated at 1.1 mL/min of A=10 mM ammonium formate adjusted to pH 3.5 in water and B=50:50 acetonitrile:methanol. Sample injections were typically 0.5 μL. Separations were effected using an Agilent (Wilmington, Del.) Zorbax Eclipse Plus C-18 (3.0×50 mm, 1.8 micron) column operating at 60° C. As samples eluted the UV detector, they were diluted in the dynamic splitter with an isocratic pump flowing at 0.5 mL/min and using a solvent of 50:50 methanol:water. Samples were diluted by factors ranging from 33:1 to 100:1. Data was processed using the MassHunter software that was provided with the instrument.

Compounds of Formula I can be prepared by a variety of methods. Reaction schemes and Examples below are representative methods for preparing compounds of Formulae I-V. Modifications of these methods should be readily apparent to those skilled in the art.

### Synthesis

Carboxylic acids used for the assembly of polymyxin derivatives were secured either via commercial sources or prepared using methods known to those skilled in the art. Experimental details of the following carboxylic acids serve as representative examples for the synthesis of similar acid intermediates used in the synthesis of the compounds of the present invention.

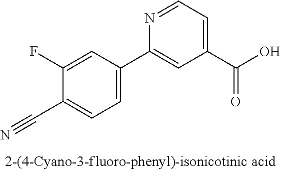

To a mixture of 4-cyano-3-fluoro-phenylboronic acid (612 mg, 3.71 mmol, 1.5 equiv.) and 2-bromo-isonicotinic acid (500 mg, 2.48 mmol, 1.0 equiv.) in dioxane (5 mL) was added 2 M Na₂CO₃ (4.34 mL, 8.68 mmol, 3.5 equiv.) and Pd(PPh₃)₄ (286 mg, 0.248 mmol, 0.1 equiv.) in a 35-mL Q-tube. The mixture was stirred at 180° C. for 20 min. Crude mixture was filtered through Celite. Celite was rinsed several times with water (2×5 mL). The mixture was poured into a separatory funnel and extracted with heptane (3×20 mL) and then with EtOAc (1×20 mL) to remove impurities. The aqueous layer was acidified to pH ˜2 with 1N HCl and the product precipitated out. The solid was collected via filtration and kept under high vacuum for 16 h to afford 420 mg crude product, which was purified via HPLC on a Phenomenex HILIC (Diol) 250×21.1 mm 5 micron column using heptane/ethanol gradient solvent system to afford 220 mg desired product (37% yield). ¹H NMR (400 MHz, DMSO-d₆) δ 7.90 (dd, J=4.98, 1.46 Hz, 1H), 8.07 (dd, J=8.20, 6.83 Hz, 1H), 8.21 (dd, J=8.20, 1.56 Hz, 1H), 8.28 (dd, J=11.03, 1.46 Hz, 1H), 8.47 (s, 1H), 8.93 (d, J=4.88 Hz, 1H), 13.89 (br. s., 1H).

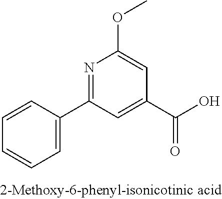

Step 1: Citrazinic acid (2.00 g, 12.9 mmol, 1.0 eq) and phosphorous oxybromide (11.19 g, 3.0 eq) were heated at 180° C. under nitrogen for 2 h. The reaction mixture was cooled to RT and ice water added cautiously and mixture was left at RT for 12 h. The mixture was extracted with EtOAc (4×100 mL). Combined organic layer was dried over sodium sulfate, filtered, and concentrated to dryness to afford 3.00 g (83%) of 2,6-dibromo-isonicotinic acid as a light brown solid.

Step 2: 2,6-Dibromo-isonicotinic acid (3.00 g, 1 eq) and sodium methoixde (2.88 g, 5 eq) were dissolved in anhydrous MeOH (21 mL) and heated to reflux for 48 h. Mixture was cooled to RT, dissolved in 20 mL water and adjusted to pH˜3 with 3 N HCl. This was then extracted with EtOAc (3×50 mL). Combined organic layer was dried over sodium sulfate, filtered, and concentrated to dryness to afford 2.3 g (93%) of 2-bromo-6-methoxy-isonicotinic acid as a light brown solid.

Step 3: To a mixture of phenylboronic acid (394 mg, 1.5 eq) and crude acid of step 2 (500 mg, 1.0 eq) in dioxane (5 mL) was added 2 M Na₂CO₃ (3.76 mL, 3.5 eq) and Pd(PPh₃)₄ (248 mg, 0.1 eq) in a 35-mL Q-tube. The mixture was stirred at 180° C. for 20 min. Crude product was filtered through Celite. Celite was rinsed several times with water (2×5 mL). The filtrate was poured into a separatory funnel and extracted with heptane (3×20 mL) and then with EtOAc (1×20 mL) to remove some of the unwanted organic materials. The aqueous layer was acidified to pH ˜2 with 1 N HCl and extracted with EtOAc (3×20 mL). Combined EtOAc layers were dried over anhydrous sodium sulfate, filtered and concentrated to dryness to afford 412 mg of crude yellow solid. The crude product was purified by flash chromatography using an Analogix SF25-40 g column with a gradient of 0-5% of MeOH/DCM. Relevant fractions were combined and concentrated to dryness to afford 200 mg (41%) of desired product as a white solid. ¹H NMR (400 MHz, DMSO-d₆) δ 13.69 (br. s., 1H), 8.06-8.21 (m, 2H), 7.90 (d, J=1.17 Hz, 1H), 7.38-7.62 (m, 3H), 7.16 (d, J=0.98 Hz, 1H), 4.01 (s, 3H).

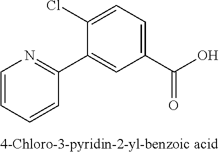

Step 1: 5-Ethoxycarbonyl-2-chlorophenyl boronic acid (2.05 g, 8.87 mmol, 1.0 equiv.) was dissolved in a solution of degassed 1,4-dioxane/water (5:1, 49.0 mL) followed by 2-chloropyridine (0.917 mL, 9.69 mmol, 1.1 equiv.), potassium carbonate (3.65 g, 26.4 mmol, 3.0 equiv.) and Pd(PPh₃)₄ (1.04 g, 0.881 mmol, 0.10 equiv.) the reaction was stirred at 83° C. for 3.0 h. The mixture was cooled to room temperature. Water (30.0 mL) and ethyl acetate (150.0 mL) were added. The organics were separated and the aqueous is extracted with ethyl acetate (3×80.0 mL). The combined organics were dried over sodium sulfate, filtered, and concentrated. The crude product was purified by column chromatography (Analogix SF-65 400 g column) using 8-50% EtOAc/heptane gradient to yield the desired product (1.01 g, 3.86 mmol, 43.9%).

Step 2: The ethyl ester (1.01 g, 3.86 mmol, 1.0 equiv.) was dissolved in a solution of water:THF (1:2, 60.0 mL) and treated with LiOH hydrate (0.277 g, 11.6 mmol, 3.0 equiv.). The solution was stirred at RT for 4.0 h, then extracted with heptanes (2×20 mL). The mixture was then saturated with sodium chloride, and pH was adjusted stepwise from 5 to 1 with 1 N HCl. This mixture was subsequently extracted with EtOAc (9×50 mL). The combined EtOAc layer was dried over sodium sulfate, filtered, and concentrated to yield the desired product 4-chloro-3-pyridin-2-yl-benzoic acid (0.650 g, 62.5%). ¹H NMR (400 MHz, DMSO-d₆) δ ppm 13.26 (br. S, 1H), 8.55-8.85 (m, 1H), 7.87-8.17 (m, 3H), 7.63-7.78 (m, 2H), 7.44 (ddd, J=7.61, 4.88, 1.17 Hz, 1H).

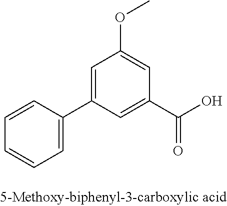

Step 1: A mixture of 1,3-dibromo-5-methoxy-benzene (5 g, 18.8 mmol), Pd(dppf)Cl₂ (4 g, 5.47 mmol) and Et₃N (3.8 g, 37.55 mmol) in MeOH/DMF (75 mL/75 mL) was stirred at 50° C. under 50 psi CO for 16 h. TLC (petroleum ether/EtOAc 10/1) indicated little starting material remained. The mixture was cooled to RT and filtered. The filtrate was concentrated and the residue was dissolved in water (100 mL), extracted with EtOAc (3×100 mL). The combined organic layer was washed with brine, dried over sodium sulfate, filtrated and concentrated. The residue was purified via flash column chromatography to give an oil product 3-bromo-5-methoxy-benzoic acid methyl ester (1.2 g, 24.5%).

Step 2: A mixture of 3-bromo-5-methoxy-benzoic acid methyl ester (830 mg, 3.39 mmol), phenylboronic acid (573 mg, 4.07 mmol), Pd(PPh₃)₄ (200 mg, 0.173 mmol) and sodium carbonate (900 mg, 8.49 mmol) in DME/water (20/20 mL) was heated to reflux with stirring for 16 h. under N2. TLC (petroleum ether/EtOAc 20/1) showed the reaction was complete. The reaction mixture was filtered and the filtrate was extracted with EtOAc (50 mL×4). The combined organic layer was washed with brine, dried over sodium sulfate and concentrated to give 5-methoxy-biphenyl-3-carboxylic acid methyl ester (1.3 g), which was purified via flash chromatography to give the product as an oil (600 mg, 73.2%).

Step 3: To a mixture of 5-methoxy-biphenyl-3-carboxylic acid methyl ester (600 mg, 2.49 mmol) in THF/water (30 mL/30 mL) was added LiOH hydrate (312 mg, 7.43 mmol) at 0-5° C. After the addition, the mixture was stirred at room temperature for 16 hrs. TLC (petroleum ether/EtOAc 10/1) showed the reaction was complete. The mixture was concentrated and the residue was dissolved in water (150 mL) and extracted with ether (3×150 mL). The aqueous layer was acidified pH to 2-3 with 1M HCl. Extracted with EtOAc (6×50 mL) and the combined organic layer was dried, filtered and concentrated to give 5-methoxy-biphenyl-3-carboxylic acid (540 mg, 95.6%) as a white solid. ¹H NMR (400 MHz, DMSO-d₆) δ ppm 13.14 (br. s, 1H), 7.70-7.77 (m, 3H), 7.39-7.49 (m, 5H), 3.88 (s, 3H).

Peptide total synthesis was carried out using standard methods to generate the selectively protected intermediate compound A, H-Dab(Boc)-Thr(tBu)-Dap(Boc)-cyclo[Dab-Dab(Boc)-DPhe-Leu-Dab(Boc)-Dab(Boc)-Thr(tBu)], from Preparation 1, which was subsequently further modified to yield the final polymyxin analogs as described below.

### Preparation of compound A: H-Dab(Boc)-Thr(tBu)-Dap(Boc)-cyclo[Dab-Dab(Boc)-DPhe-Leu-Dab (Boc)-Dab(Boc)-Thr(tBu)]

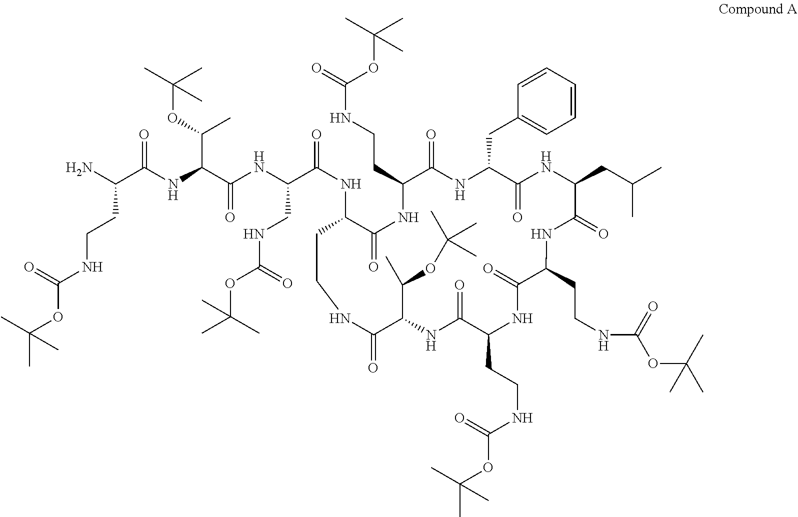

### Step 1: Fmoc-Leu-CTC Resin

CTC resin (950 g, 988 mmol) was suspended in DCM (8.0 L) in a peptide synthesis vessel equipped with a mechanical stirrer. To the suspension was added Fmoc-Leu-OH (349 g, 988 mmol) and DIPEA (863 mL, 4940 mmol). The mixture was stirred gently at RT for 3 h. To the reaction was added 0.95 L MeOH. The resulting mixture was stirred at RT for 0.5 h and then filtered. Solid was washed with DCM (3×15 L) and subsequently MeOH (2×10 L). The resulting solid was dried under vacuum overnight to furnish 1250 g of Fmoc-Leu-CTC resin.

Loading level of the resin product thus obtained was established to be 0.46 mmol/g via standard UV absorption method (Shimadzu UV-1601, wavelength 289.5 nm) upon Fmoc cleavage of small aliquots of the product resin.

### Step 2: Fmoc-DPhe-Leu-CTC Resin

Fmoc-Leu-CTC resin (152 g, 0.46 mmol/g, 70 mmol) was suspended in DMF (1.0 L) at RT overnight in a peptide synthesis vessel equipped with a mechanical stirrer. DMF was then removed via filtration. To the solid was added 20% piperidine in DMF (1.0 L) and the resulting mixture was stirred at 18° C. for 0.5 h. Mixture was then filtered. Solid resin H-Leu-CTC was washed with DMF (6×1 L).

Fmoc-DPhe-OH (54.2 g, 140 mmol) and HOBt (18.9 g, 140 mmol) were dissolved in DMF (0.25 L) at 0° C. To the mixture was added DIC (22.1 mL, 140 mmol) at 0° C. in one portion and mixture was kept at 0° C. for 15 min. The activated AA solution was then added into the H-Leu-CTC resin and the mixture was stirred at 18° C. for 1 h, at which point Kaiser ninhydrin test indicated reaction completion. The mixture was filtered and solid was washed with DMF (5×1 L). The Fmoc-DPhe-Leu-CTC resin product was used in subsequent step without further treatment.

Step 3: Fmoc-Dab(Boc)-DPhe-Leu-CTC resin

To the Fmoc-DPhe-Leu-CTC resin in a peptide synthesis vessel equipped with a mechanical stirrer, obtained from step 2, was added 20% piperidine in DMF (1.0 L) and the resulting mixture was stirred gently at 20° C. for 0.5 h. The mixture was then filtered. Solid resin H-DPhe-Leu-CTC was washed with DMF (6×1.0 L).

Fmoc-Dab(Boc)-OH (61.6 g, 140 mmol) and HOBt (18.9 g, 140 mmol) were dissolved in DMF (0.25 L) at 0° C. To the mixture was added DIC (22.1 mL, 140 mmol) at 0° C. in one portion and the mixture was kept at 0° C. for 15 min. The activated AA solution was then added into H-DPhe-Leu-CTC resin and the mixture was stirred at 19° C. for 1 h, at which point Kaiser ninhydrin test indicated reaction completion. The mixture was filtered and solid was washed with DMF (5×1 L). The resulting Fmoc-Dab(Boc)-DPhe-Leu-CTC resin product was used in subsequent step without further treatment.

Step 4: Fmoc-Dab(Dde)-Dab(Boc)-DPhe-Leu-CTC resin

To the Fmoc-Dab(Boc)-DPhe-Leu-CTC resin in a peptide synthesis vessel equipped with a mechanical stirrer, obtained from step 3, was added 20% piperidine in DMF (1.0 L) and the resulting mixture was stirred at 19° C. for 0.5 h. The mixture was then filtered. Solid H-Dab(Boc)-DPhe-Leu-CTC resin was washed with DMF (6×1.0 L).

Fmoc-Dab(Dde)-OH (70.4 g, 140 mmol) and HOBt (18.9 g, 140 mmol) were dissolved in DMF (0.25 L) at 0° C. To the mixture was added DIC (22.1 mL, 140 mmol) at 0° C. in one portion and the mixture was kept at 0° C. for 15 min. The activated AA solution was then added into H-Dab(Boc)-DPhe-Leu-CTC resin and the mixture was stirred at 21° C. for 1 h, at which point Kaiser ninhydrin test indicated reaction completion. The mixture was filtered and solid was washed with DMF (5×1 L). The resulting Fmoc-Dab(Dde)-Dab(Boc)-DPhe-Leu-CTC resin product was used in subsequent step without further treatment.

Step 5: Fmoc-Dap(Boc)-Dab(Dde)-Dab(Boc)-DPhe-Leu-CTC resin

To the resin Fmoc-Dab(Dde)-Dab(Boc)-DPhe-Leu-CTC in a peptide synthesis vessel equipped with a mechanical stirrer, obtained from the step 4, was added 20% piperidine in DMF (1.5 L) and the resulting mixture was stirred at 22° C. for 0.5 h. Mixture was then filtered. Solid H-Dab(Dde)-Dab(Boc)-DPhe-Leu-CTC resin was washed with DMF (6×1.5 L).

Fmoc-Dap(Boc)-OH (59.6 g, 140 mmol) and HOBt (18.9 g, 140 mmol) were dissolved in DMF (0.25 L) at 0° C. To the mixture was added DIC (22.1 mL, 140 mmol) at 0° C. in one portion and the mixture was kept at 0° C. for 15 minutes. The activated AA solution was then added into H-Dab(Dde)-Dab(Boc)-DPhe-Leu-CTC resin and mixture was stirred at 22° C. for 1 h, at which point Kaiser ninhydrin test indicated reaction completion. The mixture was filtered and the solid was washed with DMF (5×1 L). The resulting Fmoc-Dap(Boc)-Dab(Dde)-Dab(Boc)-DPhe-Leu-CTC resin product was confirmed by peptide cleavage and subsequent HPLC and LCMS analysis outlined below.

A small portion of the resin product (1 mL) was washed with MeOH (3×5 mL) and then dried overnight under vacuum. The resin was then treated with 5 mL of solution F at RT for 2.5 h. The reaction mixture was filtered and the filtrate was quenched with cold ether (50 mL). A white solid formed and this solid was collected via removal of the solvent after the mixture was centrifuged. The solid residue was washed with cold ether (2×50 mL), dried under vacuum overnight and analyzed using an HP1090 system connected to a SepaxGP-C18 (5 micron, 120 A, 4.6×150 mm) reversed-phase HPLC column. Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 13.8 min., 78.1% by UV220; ESI-MS (Thermo LCQ advantage): (m/z)=951.4([M+H]⁺), 476.4 ([M+2H]²⁺/2).

Step 6: Fmoc-Thr(tBu)-Dap(Boc)-Dab(Dde)-Dab(Boc)-DPhe-Leu-CTC resin

To the resin Fmoc-Dap(Boc)-Dab(Dde)-Dab(Boc)-DPhe-Leu-CTC in a peptide synthesis vessel equipped with a mechanical stirrer, obtained from step 5, was added 20% piperidine in DMF (1.5 L) and the resulting mixture was stirred kept at 21° C. for 0.5 h. The mixture was then filtered. The solid H-Dap(Boc)-Dab(Dde)-Dab(Boc)-DPhe-Leu-CTC resin was washed with DMF (6×1.5 L).

Fmoc-Thr(tBu)-OH (55.6 g, 140 mmol) and HOBt (18.9 g, 140 mmol) were dissolved in DMF (0.25 L) at 0° C. To the mixture was added DIC (22.1 mL, 140 mmol) at 0° C. in one portion and the mixture was kept at 0° C. for 15 minutes. The activated AA solution was then added into H-Dap(Boc)-Dab(Dde)-Dab(Boc)-DPhe-Leu-CTC resin and the mixture was stirred for 1 h, at which point Kaiser ninhydrin test indicated reaction completion. The mixture was filtered and the solid was washed with DMF (5×1 L). The resulting Fmoc-Thr(tBu)-Dap(Boc)-Dab(Dde)-Dab(Boc)-DPhe-Leu-CTC resin product was confirmed by peptide cleavage and subsequent HPLC and LCMS analysis outlined in step 5.

Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 19.3 min., 89.6% by UV220; ESI-MS (Thermo LCQ advantage): (m/z)=1052.5([M+H]⁺), 527.0 ([M+2H]⁺/2).

Step 7: Fmoc-Dab(Boc)-Thr(tBu)-Dap(Boc)-Dab(Dde)-Dab(Boc)-DPhe-Leu-CTC resin

To the resin Fmoc-Thr(tBu)-Dap(Boc)-Dab(Dde)-DPhe-Leu-CTC in a peptide synthesis vessel equipped with a mechanical stirrer, obtained from step 6, was added 20% piperidine in DMF (1.5 L) and the resulting mixture was stirred at 21° C. for 0.5 h. The mixture was then filtered and the resin H-Thr(tBu)-Dap(Boc)-Dab(Dde)-Dab(Boc)-DPhe-Leu-CTC was washed with DMF (6×1.5 L).

Fmoc-Dab(Boc)-OH (61.6 g, 140 mmol) and HOBt (18.9 g, 140 mmol) were dissolved in DMF (0.25 L) at 0° C. To the mixture was added DIC (22.1 mL, 140 mmol) at 0° C. in one portion and the mixture was kept at 0° C. for 15 minutes. The activated AA solution was then added into H-Thr(tBu)-Dap(Boc)-Dab(Dde)-Dab(Boc)-DPhe-Leu-CTC resin and the mixture was stirred at 22° C. for 1 h, at which point Kaiser ninhydrin test indicated reaction completion. The mixture was filtered and the solid was washed with DMF (5×1 L). The Fmoc-Dab(Boc)-Thr(tBu)-Dap(Boc)-Dab(Dde)-Dab(Boc)-DPhe-Leu-CTC resin product was confirmed by peptide cleavage and subsequent HPLC and LCMS analysis outlined in step 5.

Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 17.5 min., 78.7% by UV220; ESI-MS (Thermo LCQ advantage): (m/z)=1152.5([M+H]⁺), 577.9 ([M+2H]⁺/2).

Step 8: Z-Dab(Boc)-Thr(tBu)-Dap(Boc)-Dab(Dde)-Dab(Boc)-DPhe-Leu-CTC resin

To the resin Fmoc-Dab(Boc)-Thr(tBu)-Dap(Boc)-Dab(Dde)-DPhe-Leu-CTC in a peptide synthesis vessel equipped with a mechanical stirrer, obtained from the step 7, was added into 20% piperidine in DMF (2.0 L) and the resulting mixture was stirred at RT for 0.5 h. The mixture was then filtered and the solid H-Dab(Boc)-Thr(tBu)-Dap(Boc)-Dab(Dde)-Dab(Boc)-DPhe-Leu-CTC resin was washed with DMF (5×2.0 L). To the resin was then added a solution of Z—OSu (52.3 g, 210 mmol) and NMM (46.2 mmol, 420 mmol) in DMF (0.50 L). The resulting mixture was stirred for 1 h. Kaiser ninhydrin test indicated reaction completion. The mixture was filtered after stirred at RT for another 50 minutes. Solid was washed with DMF (5×2 L) and the product, Z-Dab(Boc)-Thr(tBu)-Dap(Boc)-Dab(Dde)-Dab(Boc)-DPhe-Leu-CTC resin product was confirmed by peptide cleavage and subsequent HPLC and LCMS analysis outlined in step 5.

Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 11.7 min., 73.7% by UV220; ESI-MS (Thermo LCQ advantage): (m/z)=1064.5([M+H]⁺), 533.0 ([M+2H]⁺/2).

Step 9: Z-Dab(Boc)-Thr(tBu)-Dap(Boc)-Dab[Thr(tBu)-Fmoc]-Dab(Boc)-DPhe-Leu-CTC resin

To the resin Z-Dab(Boc)-Thr(tBu)-Dap(Boc)-Dab(Dde)-Dab(Boc)-DPhe-Leu-CTC in a peptide synthesis vessel equipped with a mechanical stirrer, obtained from step 8, was added a solution of 3% hydrazine in DMF (2.0 L). The resulting mixture was stirred at 20° C. for 0.5 h. The mixture was then filtered and the solid Z-Dab(Boc)-Thr(tBu)-Dap(Boc)-Dab[-H]-Dab(Boc)-DPhe-Leu-CTC resin was washed with DMF (5×2.0 L).

Fmoc-Thr(tBu)-OH (55.6 g, 140 mmol) and HOBt (18.9 g, 140 mmol) were dissolved in DMF (0.30 L) at 0° C. To the mixture was added DIC (22.1 mL, 140 mmol) at 0° C. in one portion and the mixture was kept at 0° C. for 15 minutes. The activated AA solution was then added into the resin Z-Dab(Boc)-Thr(tBu)-Dap(Boc)-Dab[-H]-Dab(Boc)-DPhe-Leu-CTC and the mixture was stirred at 18° C. for 1 h at which point Kaiser ninhydrin test indicated reaction completion. The mixture was stirred for 1 more hour and then filtered. The solid was washed with DMF (5×1 L) and the resin product Z-Dab(Boc)-Thr(tBu)-Dap(Boc)-Dab[Thr(tBu)-Fmoc]-DPhe-Leu-CTC was confirmed by peptide cleavage and subsequent HPLC and LCMS analysis outlined in step 5.

Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 13.8 min., 91.1% by UV220; ESI-MS (Thermo LCQ advantage): (m/z)=1223.5([M+H]⁺), 612.6 ([M+2H]⁺/2).

Step 10: Z-Dab(Boc)-Thr(tBu)-Dap(Boc)-Dab[Thr(tBu)-Dab(Boc)-Fmoc]-Dab(Boc)-DPhe-Leu-CTC resin

To the resin Z-Dab(Boc)-Thr(tBu)-Dap(Boc)-Dab[Thr(tBu)-Fmoc]-Dab(Boc)-DPhe-Leu-CTC in a peptide synthesis vessel equipped with a mechanical stirrer, obtained from step 9, was added 20% piperidine in DMF (2.0 L) and the resulting mixture was stirred at 19° C. for 0.5 h. Mixture was then filtered and the solid Z-Dab(Boc)-Thr(tBu)-Dap(Boc)-Dab[Thr(tBu)-H)]-Dab(Boc)-DPhe-Leu-CTC resin was washed with DMF (5×2.0 L).

Fmoc-Dab(Boc)-OH (61.6 g, 140 mmol) and HOBt (18.9 g, 140 mmol) were dissolved in DMF (0.30 L) at 0° C. To the mixture was added DIC (22.1 mL, 140 mmol) at 0° C. in one portion and the mixture was kept at 0° C. for 15 min. The activated AA solution was then added into Z-Dab(Boc)-Thr(tBu)-Dap(Boc)-Dab[Thr(tBu)-H]-Dab(Boc)-DPhe-Leu-CTC and the mixture was stirred at 19° C. for 1 h at which point Kaiser ninhydrin test indicated reaction completion. The mixture was stirred for 1 more hour and then filtered. The solid was washed with DMF (5×2 L). The resin product Z-Dab(Boc)-Thr(tBu)-Dap(Boc)-Dab[Thr(tBu)-Dab(Boc)-Fmoc]-Dab(Boc)-DPhe-Leu-CTC was confirmed by peptide cleavage and subsequent HPLC and LCMS analysis outlined in step 5.

Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 16.3 min., 79.2% by UV220; ESI-MS (Thermo LCQ advantage): (m/z)=1323.5([M+H]⁺), 662.5 ([M+2H]⁺/2).

Step 11: Z-Dab(Boc)-Thr(tBu)-Dap(Boc)-Dab[Thr(tBu)-Dab(Boc)-Dab(Boc)-Fmoc]-Dab(Boc)-DPhe-Leu-CTC resin

To the resin Z-Dab(Boc)-Thr(tBu)-Dap(Boc)-Dab[Thr(tBu)-Dab(Boc)-Fmoc]-Dab(Boc)-DPhe-Leu-CTC in a peptide synthesis vessel equipped with a mechanical stirrer, obtained from step 10, was added 20% piperidine in DMF (2.0 L) and the resulting mixture was stirred at 21° C. for 0.5 h. Mixture was then filtered. Solid Z-Dab(Boc)-Thr(tBu)-Dap(Boc)-Dab[Thr(tBu)-Dab(Boc)-H)]-Dab(Boc)-DPhe-Leu-CTC resin was washed with DMF (5×2.0 L).

Fmoc-Dab(Boc)-OH (61.6 g, 140 mmol) and HOBt (18.9 g, 140 mmol) were dissolved in DMF (0.30 L) at 0° C. To the mixture was added DIC (22.1 mL, 140 mmol) at 0° C. in one portion and the mixture was kept at 0° C. for 15 minutes. The activated AA solution was then added into Z-Dab(Boc)-Thr(tBu)-Dap(Boc)-Dab[Thr(tBu)-Dab(Boc)-H]-Dab(Boc)-DPhe-Leu-CTC and mixture was stirred at 22° C. for 1 h at which point Kaiser ninhydrin test indicated reaction completion. The mixture was stirred for 1 more hour and then filtered. The solid was washed with DMF (5×2 L) and the Z-Dab(Boc)-Thr(tBu)-Dap(Boc)-Dab[Thr(tBu)-Dab(Boc)-Dab(Boc)-Fmoc]-Dab(Boc)-DPhe-Leu-CTC was confirmed by peptide cleavage and subsequent HPLC and LCMS analysis outlined in step 5.

Gradient method: 15% to 75% solvent B over 20 minutes at a flow rate of 1.0 mL/min (solvent A: 0.1% TFA in water; solvent B: 0.09% TFA, 20% water and 79.91% ACN). Major peak with retention time 14.9 min., 84.2% by UV220; ESI-MS (Thermo LCQ advantage): (m/z)=1423.6([M+H]⁺), 712.5 ([M+2H]⁺/2).

### Step 12: Z-Dab(Boc)-Thr(tBu)-Dap(Boc)-cyclo[Dab-Dab(Boc)-DPhe-Leu-Dab(Boc)-Dab(Boc)-Thr(tBu)]

To the resin Z-Dab(Boc)-Thr(tBu)-Dap(Boc)-Dab[Thr(tBu)-Dab(Boc)-Dab(Boc)-Fmoc]-Dab(Boc)-DPhe-Leu-CTC in a peptide synthesis vessel equipped with a mechanical stirrer, obtained from step 11, was added 20% piperidine in DMF (2.0 L) and the resulting mixture was stirred at 23° C. for 0.5 h. The mixture was then filtered. The solid resin Z-Dab(Boc)-Thr(tBu)-Dap(Boc)-Dab[Thr(tBu)-Dab(Boc)-Dab(Boc)-H]-Dab(Boc)-DPhe-Leu-CTC was washed with DMF (5×2.0 L).

To the resin was then added 6 L 1.5% TFA in DCM and the resulting mixture was stirred at RT for 0.5 h. The mixture was then filtered. The filtrate was neutralized with the addition of DIPEA (˜180 mL) to a pH around 7. The mixture was then concentrated to remove DCM. The residue was dissolved in 35 L DMF to make up a solution of the cleaved acyclic peptide in 0.002 M concentration. To this solution was added DIPEA (61.3 mL, 350 mmol) and EDC-HCl (26.8 g, 140 mmol). The resulting mixture was stirred at RT. LCMS indicated cyclization reaction completed after overnight stirring. DMF was removed on the rotary evaporator with water bath temperature no higher than 40° C. The residue was dissolved in DCM (1.5 L) and the DCM solution was washed with water (4×0.70 L). DCM layer was concentrated and dried under high vacuum to furnish 130 g of the desired cyclic peptide Z-Dab(Boc)-Thr(tBu)-Dap(Boc)-cyclo[Dab-Dab(Boc)-DPhe-Leu-Dab(Boc)-Dab(Boc)-Thr(tBu)]as a light yellow solid.

HPLC and LCMS analysis: gradient method: 70% to 100% B over 20 min. at a flow rate of 1.0 mL·min. (A: 0.1% TFA in water, B: 0.1% TFA in 80% ACN and 20% water). Major peak with retention time 17.24 min., 70.5% by UV220; ESI-MS (Thermo LCQ advantage): (m/z)=1795.7([M+H]⁺), 898.6 ([M+2H]⁺/2). High resolution mass spectrometry result indicated the presence of M+1, M+2, M+3 isotope distribution pattern consistent with the desired monocyclic peptide product.

### Step 13: H-Dab(Boc)-Thr(tBu)-Dap(Boc)-cyclo[Dab-Dab(Boc)-DPhe-Leu-Dab(Boc)-Dab(Boc)-Thr(tBu)]

To a stirred solution of Z-Dab(Boc)-Thr(tBu)-Dap(Boc)-cyclo[Dab-Dab(Boc)-DPhe-Leu-Dab(Boc)-Dab(Boc)-Thr(tBu)] (obtained from step 12, 130 g, assuming 70 mmol) in DCM/MeOH (1.5 L/1.5 L) in a round-bottom-flask was added concentrated ammonium hydroxide (10 mL). This mixture was then purged with N₂ and 10% Pd/C (dry, 15 g) was added under N₂. The reaction mixture was stirred at 15° C. under 1.1 atmospheric pressure H₂ for 4 days. Hydrogen source was removed and the system was purged with N₂. The mixture was then filtered through a pad of celite and filtrate was concentrated in vacuum to furnish a yellow solid, 123 g (crude yield 94%).

HPLC and LCMS analysis: gradient method: 70% to 100% solvent B over 20 minutes at a flow rate of 1.0 mL/min (solvent A: 0.1% TFA in water; solvent B: 0.09% TFA, 20% water and 79.91% ACN). Major peak with retention time 13.4 min., 78.7% by UV220; ESI-MS (Thermo LCQ advantage) (m/z)=1662.6([M+H]⁺), 831.43 ([M+2H]⁺/2).

Compound A or the decapeptide template, H-Dab(Boc)-Thr(tBu)-Dap(Boc)-cyclo[Dab-Dab(Boc)-DPhe-Leu-Dab(Boc)-Dab(Boc)-Thr(tBu)], from Preparation 1 was elaborated into compounds of Formula I via standard synthetic procedures, e.g. amide coupling, urea formation, sulfonamide formation, or reductive amination followed with removal of Boc and tBu groups via global deprotection.

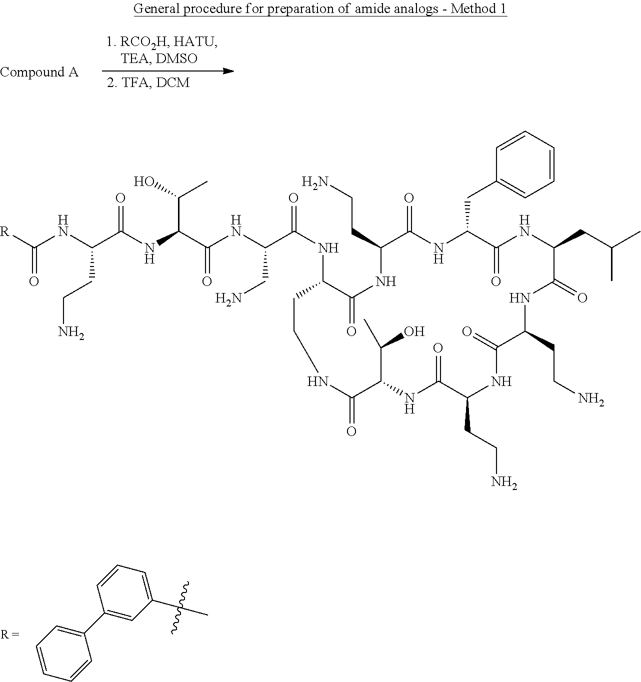

Step 1: A mixture of compound A or the decapeptide template (200 mg, 120 mmol), carboxylic acid (1.5 equiv.), HATU (2 equiv.) and TEA (4 equiv.) in DMSO (1.6 mL) was heated at 35° C. for 20 hr. LCMS indicated desired amide intermediate with mass confirmation of [M+2H]/2. The reaction mixture was purified by preparative HPLC using a Kromasil Eternity-5-C18 column (150×30 mm, 5 um), eluting with 55% to 88% B in 12 min. at a flow rate of 30 mL/min. (B: ACN with 0.1% TFA, A: H₂O with 0.1% TFA). The relevant fractions were then lyophilized to furnish the desired intermediate.
Step 2: To a solution of the intermediate (obtained in step 1) in DCM (40 mg in 4 mL) was added TFA (0.2 mL) and the mixture was stirred at RT for 4 to 5 h. LCMS indicated complete deprotection. The reaction mixture was concentrated to remove half of DCM, and then t-BME (10 mL) was added into the mixture. A white precipitate formed and was collected via filtration. The solid was dried under vacuum to afford the TFA salt of final amide target analogs, from 18 mg to 40 mg.

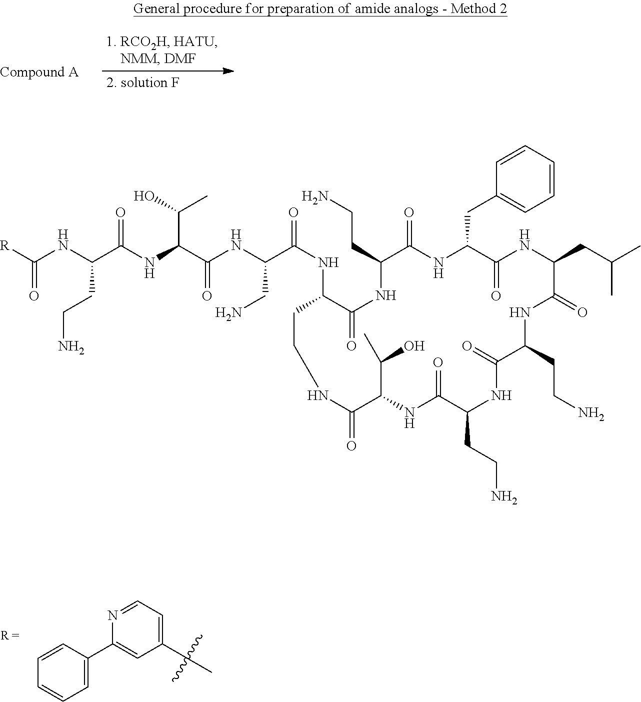

Step 1: A solution of compound A or the decapeptide template (127 g, 76.5 mmol), the acid (15.2 g, 76.5 mmol), HATU (35 g, 91.8 mmol), NMM (25.3 mL, 230 mmol) in DMF (1700 mL) in a 3 L round-bottom flask was stirred at RT overnight. TLC indicated complete consumption of the carboxylic acid starting material (DCM/MeOH/AcOH:40/1/0.5). MS confirmed reaction complete as well. The mixture was concentrated in vacuum to remove most of DMF. The residue was poured into H₂O (9 L). White solid formed. The solid was collected via filtration. The filter cake was washed with H₂O (2 L) and dried under vacuum to give 125 g of the amide intermediate as a yellow solid (89% yield), which was used in the next step without further purification.
Step 2: Into a 3 L round-bottom flask at 0° C. was charged with the yellow solid amide intermediate (125 g, 76.8 mmol) and solution F (1000 mL). The mixture was stirred at 0° C. for 0.5 h and RT for 2 h. The reaction mixture was quenched with cold ether (10 L), white solid precipitate formed and was collected by removing the supernatent after the mixture was centrifuged. This solid crude product was then purified by reversed-phase preparative HPLC to give the desired final amide target analog as a white solid TFA salt. The TFA salt thus obtained was converted to the sulfate salt by loading the TFA salt on to a Novasep-C18 10 micron 100 A 50×450 mm column and eluted, at a flow rate of 400 mL/min, initially with 90% A and 10% B for 3 column volumes, then 10% B and 90% C for 3 column volumes, and finally 40% B and 60% C for 1.5 column volumes (A: 0.2% H₂SO₄ in H₂O, B: 100% ACN, C: 100% H₂O). The fractions containing product were combined and lyophilized to furnish 24 g of a white solid (overall yield 29%). The number of equivalents of the TFA or H₂SO₄ salt was established via typical methods known to those skilled in the art of chemistry.

The carboxylic acids used in the preparation of amide analogs were either secured via commercial sources or custom synthesized using strategies and tactics known to those skilled in the art of organic synthesis.

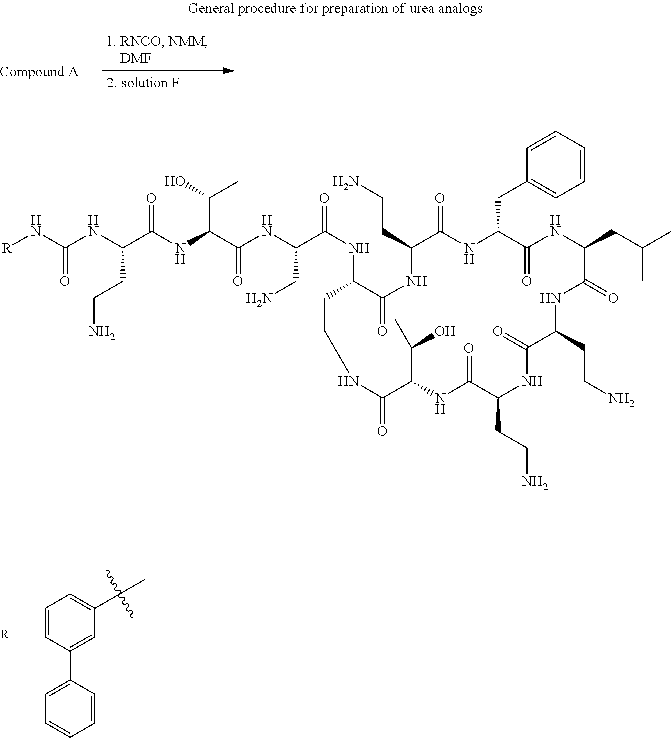

Step 1: A solution of compound A or the decapeptide template (425 mg, 0.256 mmol), the isocyanate (50 mg, 0.256 mmol), NMM (0.078 mL, 0.768 mmol) in DMF (10 mL) was stirred at RT for 4 h. MS indicated the reaction was complete. The mixture was concentrated in vacuum. The residue was dissolved in DCM (10 mL) and H₂O (10 mL). The mixture was concentrated to remove DCM. White precipitate formed and the solid was collected, dried under high vacuum to give a yellow solid (300 mg, yield 64%).
Step 2: A solution of the yellow solid (300 mg, 0.162 mmol) in F solution (3 mL) was stirred at RT for 2 h. The reaction mixture was quenched with cold ether (30 mL). White precipitate formed and the solid was isolated upon removal of the supernatant after the mixture was centrifuged. The solid was then purified by preparative HPLC to give pure product (62 mg, yield 30%) as a white solid. Preparative HPLC method: crude product dissolved in ACN/water (5/95), loaded onto a 50×250 mm 10 micron Luna C18 column and purified using a 25-50% B in 60 minutes at a flow rate of 60 mL/min (A: 0.1% TFA in water, B: ACN with 0.1% TFA). Product thus obtained was 85% pure and was re-subjected to preparative HPLC purification again using an isocratic 34% B method and the desired product was obtained in >97% purity. Analytical method: 32% B to 42% B in 20 minutes at a flow rate of 1.0 mL/minute on a SepaxGP-C18, 3 micron, 120 A, 4.6×150 mm column using UV 220 nm (Solvent A: 0.1% TFA in water, B 0.1% TFA, 20% water, 80% ACN). Final product retention time 10.5 min.; ESI-MS. (m/z)=1243.92 (100%, [M+H]⁺).

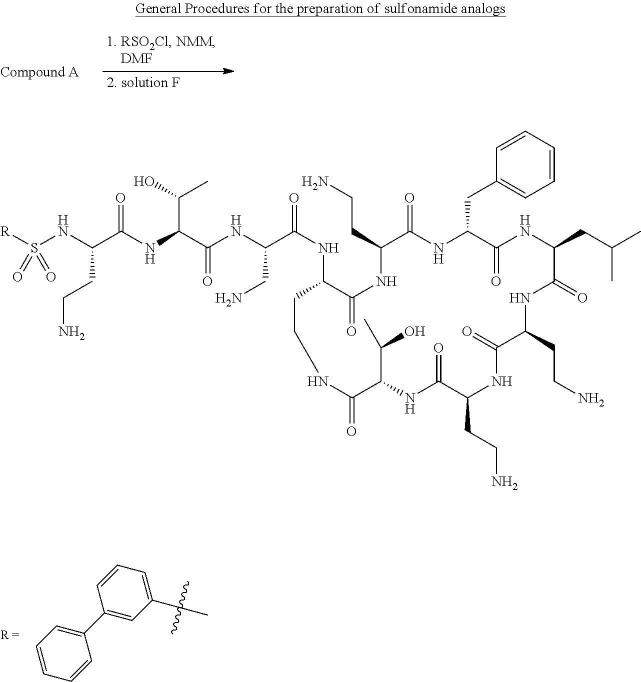

Step 1: A solution of compound A or the decapeptide template, H-Dab(Boc)-Thr(tBu)-Dab(Boc)-cyclo(Dab-Dab(Boc)-Dphe-Leu-Dab(Boc)-Dab(Boc)-Thr(tBu)) (4.37 g, 2.63 mmol), biphenyl-3-sulfonyl chloride (1.00 mg, 3.95 mmol), NMM (0.44 mL, 4.0 mmol) in DMF (40 mL) was stirred at room temperature overnight. MS spectrum indicated the reaction was complete. The mixture was poured into water (400 mL), white solid formed. The solid was collected via filtration, washed with water (50 mL×3), dried under high vacuum to give crude material as a white solid, 4.51 g (92% yield), which was used in the next step without purification.
Step 2: A solution of the white solid (4.51 g, 2.4 mmol) in F solution (40 mL) was stirred at RT for 2 hours. The reaction mixture was quenched with cold ether (400 mL), and white solid precipitate formed. The solid was isolated by removal of the supernatant after the mixture was centrifuged. The solid was dried and purified by preparative HPLC to give pure desired product as a white solid (1.394 g, yield 45%). Preparative HPLC method: crude product dissolved in ACN/water (5/95), loaded onto a 50×250 mm 10 micron Luna C18 column and purified using a 20-55% B in 60 minutes at a flow rate of 60 mL/min (A: 0.1% TFA in water, B: ACN with 0.1% TFA). Product thus obtained was 80% pure and was re-subjected to preparative HPLC purification again using an isocratic 40% B and the desired product was obtained in 97% purity. Analytical method: 33% B to 43% B in 20 minutes at a flow rate of 1.0 mL/minute on a SepaxGP-C18, 3 micron, 120 A, 4.6×150 mm column using UV 220 nm (Solvent A: 0.1% TFA in water, B 0.1% TFA, 20% water, 80% ACN). Final product retention time 7.77 min.; ESI-MS. (m/z)=633.8 (100%, [M+2H]⁺/2).

Table 1, below, lists compounds of Formula II that were prepared using synthetic procedures and/or conditions similar to those described herein.

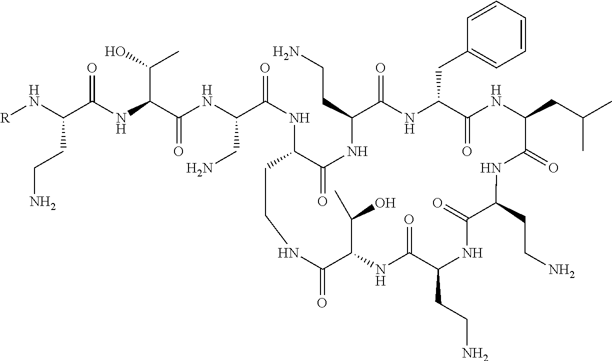

TABLE 1

| Ex. | | | |
|---|---|---|---|
| No. | R | HRMS | NMR |
| 1 | 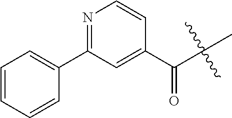 | (M + 2H)⁺² 615.8399, Calcd 615.8407. | ¹H NMR (500 MHz, D₂O) δ 8.71 (d, J = 5.20 Hz, 1H), 8.10 (d, J = 0.63 Hz, 1H), 7.89 (d, J = 7.72 Hz, 2H), 7.69 (d, J = 5.20 Hz, 1H), 7.50-7.60 (m, 3H), 7.34 (t, J = 7.30 Hz, 2H), 7.29 (t, J = 6.90 Hz, 1H), 7.21 (d, J = 7.57 Hz, 2H), 4.77-4.81 (m, 2H), 4.42-4.49 (m, 3H), 4.30-4.37 (m, 1H), 4.18-4.30 (m, 4H), 4.13 (dd, J = 3.39, 11.43 Hz, 1H), 4.09 (d, J = 4.57 Hz, 1H), 3.52 (dd, J = 4.97, 13.32 Hz, 1H), 3.28-3.39 (m, 2H), 2.99-3.21 (m, 8H), 2.89-2.99 (m, 2H), 2.81-2.89 (m, 1H), 2.32-2.43 (m, 1H), 2.10-2.32 (m, 5H), 1.89- 2.10 (m, 3H), 1.76-1.89 (m, 1H), 1.36-1.48 (m, 1H), 1.24-1.36 (m, 1H), 1.20 (d, J = 6.31 Hz, 3H), 1.13 (d, J = 6.46 Hz, 3H), 0.67 (d, J = 6.15 Hz, 3H), 0.59 (d, J = 5.67 Hz, 3H), 0.50-0.57 (m, 1H) |
| 2 | 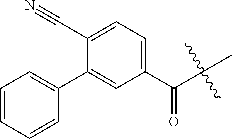 | (M + 2H)⁺² 627.8407, Calcd 627.8407. (M + 2Na)⁺² 649.8219, Calcd 649.8227. | 1H NMR (500 MHz, D₂O) δ 8.00 (d, J = 8.12 Hz, 1H), 7.96 (d, J = 1.28 Hz, 1H), 7.90 (dd, J = 1.67, 8.11 Hz, 1H), 7.65 (dd, J = 1.58, 7.87 Hz, 2H), 7.61-7.53 (m, 3H), 7.37 (t, J = 7.23 Hz, 2H), 7.32 (t, J = 7.26 Hz, 1H), 7.24 (d, J = 7.08 Hz, 2H), 4.87-4.76 (m, 2H), 4.56 (t, J = 8.25 Hz, 1H), 4.51-4.44 (m, 2H), 4.36-4.17 (m, 6H), 4.15 (d, J = 4.54 Hz, 1H), 3.51 (dd, J = 4.97, 13.43 Hz, 1H), 3.37-3.24 (m, 2H), 3.18 (t, J = 7.75 Hz, 2H), 3.14-2.99 (m, 7H), 2.91-2.83 (m, 1H), 2.82-2.72 (m, 1H), 2.41-2.29 (m, 1H), 2.29-2.08 (m, 5H), 2.07-1.77 (m, 4H), 1.53-1.44 (m, 1H), 1.43-1.33 (m, 1H), 1.20 (d, J = 6.40 Hz, 3H), 1.15 (d, J = 6.40 Hz, 3H), 0.88- 0.77 (m, 1H), 0.75 (d, J = 6.25 Hz, 3H), 0.67 (d, J = 6.19 Hz, 3H). |
| 3 | 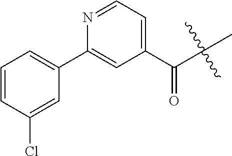 | (M + 2H)⁺² 632.8220, Calcd 632.8213. (M + 2Na)⁺² 654.8033, Calcd 654.8032. | 1H NMR (500 MHz, D₂O) δ 8.82 (d, J = 5.60 Hz, 1H), 8.31 (d, J = 0.73 Hz, 1H), 7.96 (dd, J =1.55, 5.567 Hz, 1H), 7.93 (t, J = 1.69 Hz, 1H), 7.86- 7.79 (m, 1H), 7.65-7.59 (m, 1H), 7.57 (t, J = 7.86 Hz, 1H), 7.37 (t, J = 7.23 Hz, 2H), 7.32 (t, J = 7.29 Hz, 1H), 7.24 (d, J = 7.07 Hz, 2H), 4.85- 4.78 (m, 2H), 4.55 (t, J = 8.27 Hz, 1H), 4.51- 4.45 (m, 2H), 4.38-4.12 (m, 7H), 3.52 (dd, J = 4.97, 13.44 Hz, 1H), 3.39-3.27 (m, 2H), 3.25- 2.99 (m, 9H), 2.94-2.83 (m, 1H), 2.83-2.73 (m, 1H), 2.43-2.32 (m, 1H), 2.31-2.08 (m, 5H), 2.08-1.79 (m, 4H), 1.52-1.42 (m, 1H), 1.42-1.33 (m, 1H), 1.23 (d, J = 6.40 Hz, 3H), 1.15 (d, J = 6.41 Hz, 3H), 0.84-0.76 (m, 1H), 0.74 (d, J = 5.92 Hz, 3H), 0.67 (d, J = 6.03 Hz, 3H). |
| 4 | 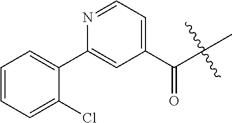 | (M + 2H)⁺² 632.8214, Calcd 632.8213. (M + 2Na⁾⁺² 654.8023, Calcd 654.8032. | 1H NMR (500 MHz, D₂O) δ 8.86 (dd, J = 0.58, 5.55 Hz, 1H), 8.19-8.11 (m, 1H), 7.99 (dd, J = 1.67, 5.55 Hz, 1H), 7.69-7.61 (m, 1H), 7.60- 7.48 (m, 3H), 7.37 (t, J = 7.23 Hz, 2H), 7.32 (t, J = 7.29 Hz, 1H), 7.24 (d, J = 7.07 Hz, 2H), 4.85- 4.76 (m, 2H), 4.55 (t, J = 8.26 Hz, 1H), 4.52- 4.44 (m, 2H), 4.36-4.12 (m, 7H), 3.51 (dd, J = 4.98, 13.43 Hz, 1H), 3.38-3.26 (m, 2H), 3.24- 2.99 (m, 9H), 2.92-2.83 (m, 1H), 2.82-2.73 (m, 1H), 2.41-2.30 (m, 1H), 2.30-2.09 (m, 5H), 2.09-1.79 (m, 4H), 1.52-1.43 (m, 1H), 1.43-1.34 (m, 1H), 1.21 (d, J = 6.40 Hz, 3H), 1.16 (d, J = 6.40 Hz, 3H), 0.86-0.76 (m, 1H), 0.74 (d, J = 6.12 Hz, 3H), 0.67 (d, J = 6.11 Hz, 3H). |
| 5 | 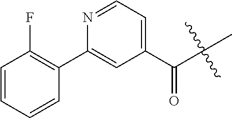 | (M + 2H)⁺² 624.8374, Calcd 624.8360. (M + 2Na)⁺² 646.8190, Calcd 646.8180. | 1H NMR (500 MHz, D₂O) δ 8.89 (d, J = 5.73 Hz, 1H), 8.33 (s, 1H), 8.05 (dd, J = 1.62, 5.69 Hz, 1H), 7.76 (td, J = 1.58, 7.75 Hz, 1H), 7.70-7.61 (m, 1H), 7.43 (td, J = 0.90, 7.66 Hz, 1H), 7.40- 7.35 (m, 3H), 7.32 (t, J = 7.30 Hz, 1H), 7.24 (d, J = 7.10 Hz, 2H), 4.86-4.76 (m, 2H), 4.55 (t, J = 8.26 Hz, 1H), 4.52-4.45 (m, 2H), 4.37-4.12 (m, 7H), 3.51 (dd, J = 4.92, 13.44 Hz, 1H), 3.38- 3.27 (m, 2H), 3.25-3.00 (m, 9H), 2.93-2.83 (m, 1H), 2.83-2.73 (m, 1H), 2.41-2.31 (m, 1H), 2.31-2.08 (m, 5H), 2.08-1.79 (m, 4H), 1.52-1.43 (m, 1H), 1.43-1.52 (m, 1H), 1.22 (d, J = 6.40 Hz, 3H), 1.16 (d, J = 6.41 Hz, 3H), 0.88- 0.76 (m, 1H), 0.74 (d, J = 5.98 Hz, 3H), 0.67 (d, J = 6.04 Hz, 3H). |
| 6 | 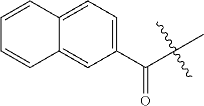 | (M + 2H)⁺² 602.3358, Calcd 602.3353. (M + Na)⁺ 1225.6447, Calcd 1225.6452. | 1H NMR (500 MHz, D₂O) δ 8.41 (s, 1H), 8.09- 8.03 (m, 2H), 8.01 (d, J = 8.03 Hz, 1H), 7.85 (dd, J = 1.62, 8.63 Hz, 1H), 7.74-7.61 (m, 2H), 7.37 (t, J = 7.25 Hz, 2H), 7.32 (t, J = 7.29 Hz, 1H), 7.24 (d, J = 7.15 Hz, 2H), 4.84-4.77 (m, 2H), 4.55 (t, J = 8.24 Hz, 1H), 4.51-4.43 (m, 2H), 4.38-4.30 (m, 1H), 4.30-4.22 (m, 3H), 4.22- 4.15 (m, 2H), 4.13 (d, J = 4.53 Hz, 1H), 3.52 (dd, J = 4.96, 13.43 Hz, 1H), 3.39-3.26 (m, 2H), 3.26-2.98 (m, 9H), 2.93-2.82 (m, 1H), 2.81- 2.72 (m, 1H), 2.45-2.34 (m, 1H), 2.34-2.06 (m, 5H), 2.06-1.78 (m, 4H), 1.52-1.43 (m, 1H), 1.42-1.34 (m, 1H), 1.22 (d, J = 6.39 Hz, 3H), 1.12 (d, J = 6.39 Hz, 3H), 0.91-0.76 (m, 1H), 0.75 (d, J = 6.28 Hz, 3H), 0.67 (d, J = 6.19 Hz, 3H). |
| 7 | 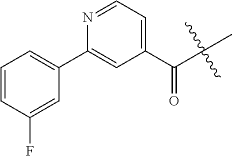 | (M + 2H)⁺² 624.8352, Calcd 624.8360. (M + 2Na)⁺² 646.8184, Calcd 646.8180. | 1H NMR (500 MHz, D₂O) δ 8.85 (dd, J = 0.55, 5.67 Hz, 1H), 8.38 (d, J = 0.84 Hz, 1H), 8.02 (dd, J = 1.63, 5.67 Hz, 1H), 7.77-7.70 (m, 1H), 7.70- 7.59 (m, 2H), 7.43-7.34 (m, 3H), 7.32 (t, J = 7.30 Hz, 1H), 7.24 (d, J = 7.06 Hz, 2H), 4.86- 4.77 (m, 2H), 4.55 (t, J = 8.27 Hz, 1H), 4.51- 4.45 (m, 2H), 4.37-4.11 (m, 7H), 3.51 (dd, J = 4.98, 13.42 Hz, 1H), 3.39-3.26 (m, 2H), 3.26- 2.98 (m, 9H), 2.94-2.83 (m, 1H), 2.83-2.72 (m, 1H), 2.42-2.32 (m, 1H), 2.32-2.08 (m, 5H), 2.08-1.79 (m, 4H), 1.52-1.42 (m, 1H), 1.42-1.32 (m, 1H), 1.22 (d, J = 6.41 Hz, 3H), 1.16 (d, J = 6.42 Hz, 3H), 0.85-0.75 (m, 1H), 0.74 (d, J = 5.61 Hz, 3H), 0.67 (d, J = 5.91 Hz, 3H). |
| 8 | 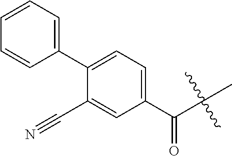 | (M + 2H)⁺² 627.8409, Calcd 627.8407. (M + 2Na)⁺² 649.8210, Calcd 649.8227. | 1H NMR (500 MHz, D₂O) δ 8.29 (d, J = 1.78 Hz, 1H), 8.13 (dd, J = 1.91, 8.21 Hz, 1H), 7.74 (d, J = 8.24 Hz, 1H), 7.65 (dd, J = 1.75, 7.73 Hz, 2H), 7.62-7.55 (m, 3H), 7.36 (t, J = 7.21 Hz, 2H), 7.31 (t, J = 7.26 Hz, 1H), 7.23 (d, J = 7.07 Hz, 2H), 4.85-4.77 (m, 2H), 4.55 (t, J = 8.24 Hz, 1H), 4.51-4.44 (m, 2H), 4.38-4.12 (m, 7H), 3.52 (dd, J = 5.00, 13.43 Hz, 1H), 3.38-3.26 (m, 2H), 3.25-2.99 (m, 9H), 2.94-2.83 (m, 1H), 2.83-2.72 (m, 1H), 2.42-2.31 (m, 1H), 2.31- 2.08 (m, 5H), 2.08-1.79 (m, 4H), 1.52-1.42 (m, 1H), 1.42-1.33 (m, 1H), 1.22 (d, J = 6.38 Hz, 3H), 1.15 (d, J = 6.41 Hz, 3H), 0.90-0.76 (m, 1H), 0.74 (d, J = 6.24 Hz, 3H), 0.67 (d, J = 6.18 Hz, 3H). |
| 9 | 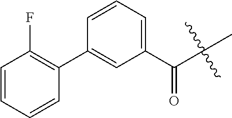 | (M + 2H)⁺² 624.3391, Calcd 624.3384. (M + Na)⁺ 1269.6523, Calcd 1269.6515. | 1H NMR (500 MHz, D₂O) δ 7.99 (d, J = 1.26 Hz, 1H), 7.87-7.80 (m, 2H), 7.64 (t, J = 7.81 Hz, 1H), 7.55 (td, J = 1.67, 7.90 Hz, 1H), 7.49-7.42 (m, 1H), 7.37 (t, J = 7.23 Hz, 2H), 7.35-7.26 (m, 3H), 7.24 (d, J = 7.08 Hz, 2H), 4.83-4.77 (m, 2H), 4.56 (t, J = 8.25 Hz, 1H), 4.50-4.43 (m, 2H), 4.38-4.29 (m, 1H), 4.29-4.16 (m, 5H), 4.15 (d, J = 4.57 Hz, 1H), 3.52 (dd, J = 5.00, 13.45 Hz, 1H), 3.38-3.25 (m, 2H), 3.24-2.99 (m, 9H) , 2.93-2.81 (m, 1H), 2.81-2.70 (m, 1H), 2.41-2.30 (m, 1H), 2.30-2.06 (m, 5H), 2.06-1.77 (m, 4H), 1.52-1.43 (m, 1H), 1.43- 1.32 (m, 1H), 1.21 (d, J = 6.41 Hz, 3H), 1.14 (d, J = 6.40 Hz, 3H), 0.87-0.77 (m, 1H), 0.75 (d, J = 6.37 Hz, 3H), 0.68 (d, J = 6.26 Hz, 3H). |
| 10 | 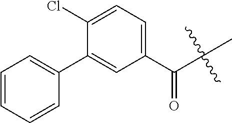 | (M + 2H)⁺² 632.3241, Calcd 632.3236. (M + 2Na)⁺² 654.3070, Calcd 654.3056. | 1H NMR (500 MHz, D₂O) δ 7.80 (d, J = 2.17 Hz, 1H), 7.77 (dd, J = 2.26, 8.37 Hz, 1H), 7.68 (d, J = 8.36 Hz, 1H), 7.56-7.46 (m, 5H), 7.37 (t, J = 7.23 Hz, 2H), 7.32 (t, J = 7.28 Hz, 1H), 7.24 (d, J = 7.09 Hz, 2H), 4.83-4.76 (m, 2H), 4.56 (t, J = 8.26 Hz, 1H), 4.51-4.43 (m, 2H), 4.36-4.17 (m, 6H), 4.15 (d, J = 4.56 Hz, 1H), 3.51 (dd, J = 4.98, 13.44 Hz, 1H), 3.38-3.25 (m, 2H), 3.22- 2.99 (m, 9H), 2.92-2.82 (m, 1H), 2.82-2.71 (m, 1H), 2.39-2.28 (m, 1H), 2.28-2.07 (m, 5H), 2.07-1.77 (m, 4H), 1.53-1.43 (m, 1H), 1.43-1.33 (m, 1H), 1.19 (d, J = 6.41 Hz, 3H), 1.15 (d, J = 6.40 Hz, 3H), 0.89-0.77 (m, 1H), 0.75 (d, J = 6.32 Hz, 3H), 0.68 (d, J = 6.22 Hz , 3H). |
| 11 | 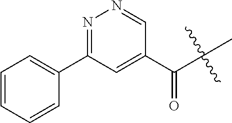 | (M + 2H)⁺² 616.3385, Calcd 616.3384. (M + 2Na)⁺² 638.3199, Calcd 638.3203. | ¹H NMR (500 MHz, D₂O) δ 9.40 (d, J = 1.98 Hz, 1H), 8.44 (d, J = 1.99 Hz, 1H), 8.04-7.90 (m, 2H), 7.65-7.53 (m, 3H), 7.32 (t, J = 7.24 Hz, 2H), 7.27 (t, J = 7.27 Hz, 1H), 7.19 (d, J = 7.09 Hz, 2H), 4.79-4.72 (m, 2H), 4.51 (t, J = 8.25 Hz, 1H), 4.45-4.39 (m, 2H), 4.31-4.06 (m, 7H), 3.47 (dd, J = 4.99, 13.41 Hz, 1H), 3.32-3.21 (m, 2H), 3.20-2.93 (m, 9H), 2.87-2.77 (m, 1H), 2.77-2.67 (m, 1H), 2.239-2.27 (m, 1H), 2.27- 2.02 (m, 5H), 2.02-1.74 (m, 4H), 1.48-1.38 (m, 1H), 1.38-1.29 (m, 1H), 1.17 (d, J = 6.40 Hz, 3H), 1.10 (d, J = 6.40 Hz, 3H), 0.83-0.72 (m, 1H), 0.70 (d, J = 6.37 Hz, 3H), 0.62 (d, J = 6.25 Hz, 3H). |
| 12 | 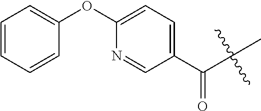 | (M + 2H)⁺² 623.8382, Calcd 623.8382. (M + Na)⁺ 1268.6505, Calcd 1268.6511. | 1H NMR (500 MHz, D₂O) δ 8.51 (d, J = 2.12 Hz, 1H), 8.22 (dd, J = 2.50, 8.76 Hz, 1H), 7.51 (t, J = 7.98 Hz, 2H), 7.42-7.28 (m, 4H), 7.25 (d, J = 7.09 Hz, 2H), 7.20 (d, J = 7.66 Hz, 2H), 7.11 (d, J = 8.77 Hz, 1H), 4.84-4.78 (m, 2H), 4.56 (t, J = 8.27 Hz, 1H), 4.48 (d, J = 5.47, 8.93 Hz, 1H), 4.45 (d, J = 3.86 Hz, 1H), 4.36-4.11 (m, 7H), 3.51 (dd, J = 4.94, 13.43 Hz, 1H), 3.37-3.25 (m, 2H), 3.22-2.98 (m, 9H), 2.93-2.82 (m, 1H), 2.82-2.72 (m, 1H), 2.40-2.28 (m, 1H), 2.27- 2.09 (m, 5H), 2.07-1.78 (m, 4H), 1.54-1.43 (m, 1H), 1.43-1.34 (m, 1H), 1.20 (d, J = 6.40 Hz, 3H), 1.17 (d, J = 6.40 Hz, 3H), 0.87-0.77 (m, 1H), 0.75 (d, J = 6.16 Hz, 3H), 0.67 (d, J = 6.15 Hz, 3H). |
| 13 | 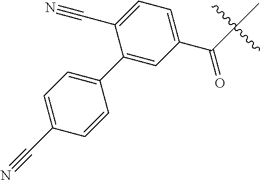 | (M + 2H)⁺² 640.3384, Calcd 640.3384. (M + 2Na)⁺² 662.3206, Calcd 662.3203. | 1H NMR (500 MHz, D₂O) δ 8.03 (d, J = 8.06 Hz, 1H), 8.00-7.94 (m, 2H), 7.92 (d, J = 8.61 Hz, 2H), 7.78 (d, J = 8.55 Hz, 2H), 7.37 (t, J = 7.21 Hz, 2H), 7.32 (t, J = 7.28 Hz, 1H), 7.24 (d, J = 7.04 Hz, 2H), 4.83-4.76 (m, 2H), 4.55 (t, J = 8.26 Hz, 1H), 4.51-4.43 (m, 2H), 4.36-4.11 (m, 7H), 3.51 (dd, J = 4.99, 13.43 Hz, 1H), 3.39- 3.25 (m, 2H), 3.24-2.98 (m, 9H), 2.93-2.83 (m, 1H), 2.83-2.73 (m, 1H), 2.40-2.29 (m, 1H), 2.30-2.09 (m, 5H), 2.07-1.76 (m, 4H), 1.53-1.42 (m, 1H), 1.42-1.33 (m, 1H), 1.20 (d, J = 6.41 Hz, 3H), 1.15 (d, J = 6.42 Hz, 3H), 0.85- 0.76 (m, 1H), 0.74 (d, J = 5.89 Hz, 3H), 0.66 (d, J = 6.01 Hz, 3H). |
| 14 | 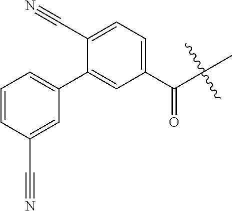 | (M + 2H)⁺² 640.3389, Calcd 640.3384. (M + Na)⁺ 1301.6494, Calcd 1301.6514. | 1H NMR (500 MHz, D₂O) δ 8.03 (d, J = 7.91 Hz, 1H), 8.00-7.87 (m, 5H), 7.71 (t, J = 7.86 Hz, 1H), 7.37 (t, J = 7.27 Hz, 2H), 7.32 (t, J = 7.21 Hz, 1H), 7.24 (d, J = 7.17 Hz, 2H), 4.83-4.77 (m, 2H), 4.55 (t, J = 8.23 Hz, 1H), 4.51-4.44 (m, 2H), 4.36-4.11 (m, 7H), 3.51 (dd, J = 4.99, 13.43 Hz, 1H), 3.39-3.24 (m, 2H), 3.23-2.98 (m, 9H), 2.93-2.82 (m, 1H), 2.82-2.72 (m, 1H), 2.40-2.30 (m, 1H), 2.30-2.08 (m, 5H), 2.08-1.75 (m, 4H), 1.53-1.43 (m, 1H), 1.43- 1.34 (m, 1H), 1.21 (d, J = 6.39 Hz, 3H), 1.15 (d, J = 6.38 Hz, 3H), 0.87-0.77 (m, 1H), 0.75 (d, J = 6.25 Hz, 3H), 0.67 (d, J = 6.18 Hz, 3H). |
| 15 | 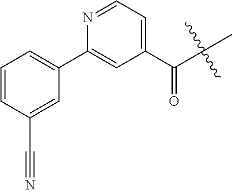 | (M + 2H)⁺² 628.3388, Calcd 628.3384. (M + Na)⁺ 1277.6511, Calcd 1277.6514. | 1H NMR (500 MHz, D₂O) δ 8.80 (dd, J = 0.43, 5.27 Hz, 1H), 8.25 (s, 1H), 8.22-8.16 (m, 2H), 7.89 (d, J = 7.83 Hz, 1H), 7.83 (dd, J = 1.52, 5.28 Hz, 1H), 7.71 (t, J =7.89 Hz, 1H), 7.37 (t, J = 7.22 Hz, 2H), 7.32 (t, J = 7.28 Hz, 1H), 7.23 (d, J = 7.06 Hz, 2H), 4.85-4.77 (m, 2H), 4.55 (t, J = 8.25 Hz, 1H), 4.51-4.45 (m, 2H), 4.38-4.10 (m, 7H), 3.52 (dd, J = 4.99, 13.43 Hz, 1H), 3.37- 3.25 (m, 2H), 3.25-2.98 (m, 9H), 2.93-2.83 (m, 1H), 2.83-2.72 (m, 1H), 2.42-2.32 (m, 1H), 2.32-2.09 (m, 5H), 2.09-1.79 (m, 4H), 1.52-1.43 (m, 1H), 1.43-1.33 (m, 1H), 1.23 (d, J = 6.40 Hz, 3H), 1.15 (d, J = 6.41 Hz, 3H), 0.86- 0.76 (m, 1H), 0.74 (d, J = 6.10 Hz, 3H), 0.67 (d, J = 6.11 Hz, 3H). |
| 16 | 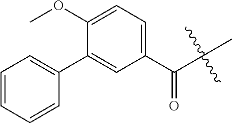 | (M + 2H)⁺² 630.3493, Calcd 630.3484. (M + Na)⁺ 1281.6722, Calcd 1281.6715. | 1H NMR (500 MHz, D₂O) δ 7.87 (dd, J = 2.40, 8.69 Hz, 1H), 7.78 (d, J = 2.39 Hz, 1H), 7.57- 7.47 (m, 4H), 7.47-7.41 (m, 1H), 7.37 (t, J = 7.23 Hz, 2H), 7.32 (t, J = 7.30 Hz, 1H), 7.24 (d, J = 8.64 Hz, 3H), 4.84-4.77 (m, 2H), 4.56 (t, J = 8.25 Hz, 1H), 4.51-4.41 (m, 2H), 4.37-4.29 (m, 1H), 4.29-4.23 (m, 3H), 4.23-4.16 (m, 2H), 4.15 (d, J = 4.58 Hz, 1H), 3.87 (s, 3H), 3.51 (dd, J = 4.99, 13.44 Hz, 1H), 3.40-3.26 (m, 2H), 3.23-2.98 (m, 9H), 2.93-2.82 (m, 1H), 2.82- 2.71 (m, 1H), 2.39-2.29 (m, 1H), 2.29-2.07 (m, 5H), 2.07-1.79 (m, 4H), 1.53-1.43 (m, 1H), 1.43-1.34 (m, 1H), 1.20 (d, J = 6.41 Hz, 3H), 1.14 (d, J = 6.40 Hz, 3H), 0.89-0.77 (m. 1H), 0.75 (d, J = 6.32 Hz, 3H), 0.68 (d, J = 6.22 Hz, 3H). |
| 17 | 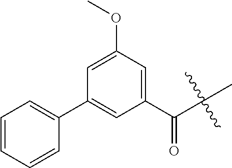 | (M + 2H)⁺² 630.3494, Calcd 630.3484. (M + 2Na)⁺² 652.3317, Calcd 652.3303. | 1H NMR (500 MHz, D₂O) δ 7.71 (d, J = 7.21 Hz, 2H), 7.68-7.65 (m, 1H), 7.53 (t, J = 7.51 Hz, 2H),l 7.50-7.44 (m, 2H), 7.40-7.34 (m, 3H), 7.32 (t, J = 7.28 Hz, 1H), 7.24 (d, J = 7.08 Hz, 2H), 4.83-4.76 (m, 2H), 4.55 (t, J = 8.25 Hz, 1H), 4.49-4.43 (m, 2H), 4.38-4.30 (m, 1H), 4.30-4.22 (m, 3H), 4.22-4.15 (m, 2H), 4.13 (d, J = 4.51 Hz, 1H), 3.93 (s, 3H), 3.52 (dd, J = 5.02, 13.43 Hz, 1H), 3.37-3.26 (m, 2H), 3.23-2.98 (m, 9H), 2.92-2.83 (m, 1H), 2.83-2.73 (m, 1H), 2.42-2.31 (m, 1H), 2.31-2.08 (m, 5H), 2.07-1.78 (m, 4H), 1.51-1.42 (m, 1H), 1.42- 1.33 (m, 1H), 1.22 (d, J = 6.41 Hz, 3H), 1.13 (d, J = 6.41 Hz, 3H), 0.85-0.76 (m, 1H), 0.74 (d, J = 6.02 Hz, 3H), 0.67 (d, J = 6.05 Hz, 3H). |
| 18 | 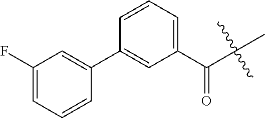 | (M + 2H)⁺² 624.3381, Calcd 624.3384. (M + Na)⁺ 1269.6480, Calcd 1269.6515. | 1H NMR (500 MHz, D₂O) δ 8.05 (s, 1H), 7.91 (d, J = 7.80 Hz, 1H), 7.82 (d, J = 8.28 Hz, 1H), 7.63 (t, J = 7.80 Hz, 1H), 7.56-7.49 (m, 2H), 7.47 (d, J = 10.38 Hz, 1H), 7.37 (t, J = 7.23 Hz, 2H), 7.32 (t, J = 7.28 Hz, 1H), 7.24 (d, J = 7.09 Hz, 2H), 7.22-7.15 (m, 1H), 4.83-4.76 (m, 2H), 4.56 (t, J = 8.24 Hz, 1H), 4.51-4.42 (m, 2H), 4.38- 4.29 (m, 1H), 4.30-4.16 (m, 5H), 4.14 (d, J = 4.54 Hz, 1H), 3.52 (dd, J = 5.00, 13.43 Hz, 1H), 3.39-03.25 (m, 2H), 3.25-2.99 (m, 9H), 2.92- 2.82 (m, 1H), 2.82-2.71 (m, 1H), 2.42-2.31 (m, 1H), 2.31-2.06 (m, 5H), 2.06-1.77 (m, 4H), 1.53-1.43 (m, 1H), 1.43-1.33 (m, 1H), 1.21 (d, J = 6.41 Hz, 3H), 1.14 (d, J = 6.40 Hz, 3H), 0.89-0.77 (m, 1H), 0.75 (d, J = 6.35 Hz, 3H), 0.67 (d, J = 6.25 Hz, 3H). |
| 19 | 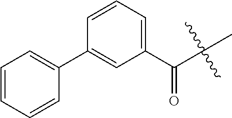 | (M + 2H)⁺² 615.3439, Calcd 615.3431. (M + Na)⁺ 1251.6610, Calcd 1251.6609. | 1H NMR (500 MHz, D₂O) δ 8.08-8.04 (m, 1H), 7.95-7.89 (m, 1H), 7.83-7.78 (m, 1H), 7.76- 7.70 (m, 2H), 7.63 (t, J = 7.81 Hz, 1H), 7.55 (t, J = 7.59 Hz, 2H), 7.47 (t, J = 7.39 Hz, 1H), 7.37 (t, J = 7.22 Hz, 2H), 7.32 (t, J = 7.30 Hz, 1H), 7.24 (d, J = 7.06 Hz, 2H), 4.83-4.76 (m, 2H), 4.56 (t, J = 8.25 Hz, 1H), 4.50-4.42 (m, 2H), 4.38- 4.29 (m, 1H), 4.29-4.16 (m, 5H), 4.14 (d, J = 4.55 Hz, 1H), 3.52 (dd, J = 4.99, 13.43 Hz, 1H), 3.38-3.25 (m, 2H), 3.24-2.98 (m, 9H), 2.92- 2.82 (m, 1H), 2.81-2.71 (m, 1H), 2.42-2.31 (m, 1H), 2.31-2.07 (m, 5H), 2.06-1.77 (m, 4H), 1.52-1.43 (m, 1H), 1.43-1.34 (m, 1H), 1.21 (d, J = 6.42 Hz, 3H), 1.14 (d, J = 6.40 Hz, 3H), 0.88-0.77 (m, 1H), 0.75 (d, J = 6.36 Hz, 3H), 0.68 (d, J = 6.26 Hz, 3H). |
| 20 | 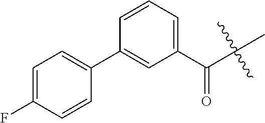 | (M + 2H)⁺² 624.3391, Calcd 624.3384. (M + Na)⁺ 1269.6508, Calcd 1269.6515. | 1H NMR (500 MHz, D₂O) δ 8.02 (s, 1H), 7.88 (d, J = 7.79 Hz, 1H), 7.79 (d, J = 8.17 Hz, 1H), 7.74- 7.67 (m, 2H), 7.62 (t, J = 7.80 Hz, 1H), 7.37 (t, J = 7.24 Hz, 2H), 7.32 (t, J = 7.28 Hz, 1H), 7.29- 7.21 (m, 4H), 4.84-4.76 (m, 2H), 4.56 (t, J = 8.24 Hz, 1H), 4.51-4.42 (m, 2H), 4.37-4.29 (m, 1H), 4.29-4.16 (m, 5H), 4.14 (d, J = 4.55 Hz, 1H), 3.52 (dd, J = 4.98, 13.43 Hz, 1H), 3.38- 3.25 (m, 2H), 3.25-2.98 (m, 9H), 2.92-2.82 (m, 1H), 2.81-2.71 (m, 1H), 2.42-2.31 (m, 1H), 2.31-2.06 (m, 5H), 2.06-1.77 (m, 4H), 1.52-1.43 (m, 1H), 1.43-1.34 (m, 1H), 1.21 (d, J = 6.41 Hz, 3H), 1.14 (d, J = 6.40 Hz, 3H), 0.88- 0.77 (m, 1H), 0.75 (d, J = 6.35 Hz, 3H), 0.67 (d, J = 6.25 Hz, 3H). |
| 21 | 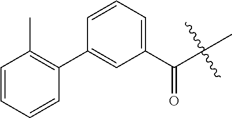 | (M + 2H)⁺² 622.3513, Calcd 622.3509. (M + Na)⁺ 1265.6761, Calcd 1265.6765. | 1H NMR (500 MHz, D₂O) δ 7.84-7.79 (m, 1H), 7.75 (s, 1H), 7.65-7.58 (m, 2H), 7.42- 7.28 (m, 7H), 7.24 (d, J = 7.09 Hz, 2H), 4.83- 4.77 (m, 2H), 4.56 (t, J = 8.25 Hz, 1H), 4.50- 4.43 (m, 2H), 4.36-4.17 (m, 6H), 4.16 (d, J = 4.59 Hz, 1H), 3.51 (dd, J = 4.97, 13.45 Hz, 1H), 3.37-3.26 (m, 2H), 3.25-2.99 (m, 9H), 2.93- 2.82 (m, 1H), 2.81-2.70 (m, 1H), 2.40-2.29 (m, 1H), 2.29-2.06 (m, 8H), 2.06-1.78 (m, 4H), 1.53-1.43 (m, 1H), 1.43-1.34 (m, 1H), 1.20 (d, J = 6.41 Hz, 3H), 1.15 (d, J = 6.39 Hz, 3H), 0.89-0.78 (m, 1H), 0.75 (d, J = 6.38 Hz, 3H), 0.68 (d, J = 6.27 Hz, 3H). |
| 22 | 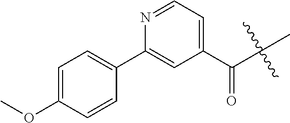 | (M + 2H)⁺² 630.8468, Calcd 630.8460. (M + Na)⁺ 1282.6658, Calcd 1282.6667. | 1H NMR (500 MHz, D₂O) δ 8.81 (d, J = 6.16 Hz, 1H), 8.54 (d, J = 1.00 Hz, 1H), 8.14 (dd, J = 1.68, 6.19 Hz, 1H), 7.92 (d, J = 8.96 Hz, 2H), 7.37 (t, J = 7.23 Hz, 2H), 7.32 (t, J = 7.28 Hz, 1H), 7.27- 7.20 (m, 4H), 4.88-4.78 (m, 2H), 4.55 (t, J = 8.25 Hz, 1H), 4.52-4.46 (m, 2H), 4.37-4.14 (m, 7H), 3.93 (s, 3), 3.51 (dd, J = 4.95, 13.44 Hz, 1H), 3.38-3.26 (m, 2H), 3.25-2.98 (m, 9H), 2.93-2.83 (m, 1H), 2.82-2.73 (m, 1H), 2.42-2.09 (m, 6H), 2.08-1.80 (m, 4H), 1.53- 1.42 (m, 1H), 1.42-1.33 (m, 1H), 1.22 (d, J = 6.32 Hz, 3H), 1.16 (d, J = 6.41 Hz, 3H), 0.85- 0.75 (m, 1H), 0.74 (d, J = 5.75 Hz, 3), 0.67 (d, J = 5.94 Hz, 3H). |
| 23 | 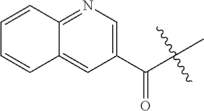 | (M + 2H)⁺² 602.8334, Calcd 602.8329. (M + 2Na)⁺² 624.8160, Calcd 624.8149. | 1H NMR (500 MHz, D₂O) δ 9.39 (d, J = 2.02 Hz, 1H), 9.29 (s, 1H), 8.30 (d, J = 8.16 Hz, 1H), 8.24 (d, J = 8.56 Hz, 1H), 8.19-8.12 (m, 1H), 7.95 (t, J = 7.33 Hz, 1H), 7.37 (t, J = 7.23 Hz, 2H), 7.32 (t, J = 7.32 Hz, 1H), 7.24 (d, J = 7.06 Hz, 2), 4.88-4.80 (m, 2H), 4.56 (t, J = 8.26 Hz, 1H), 4.52-4.45 (m, 2H), 4.37-4.10 (m, 7H), 3.52 (dd, J = 4.97, 13.45 Hz, 1H), 3.38-3.27 (m, 2H), 3.23 (t, J = 7.83 Hz, 2H), 3.18-2.99 (m, 7H), 2.93-2.82 (m, 1H), 2.83-2.71 (m, 1H), 2.45- 2.07 (m, 6H), 2.07-1.79 (m, 4H), 1.53-1.43 (m, 1H), 1.43-1.32 (m, 1H), 1.23 (d, J = 6.41 Hz, 3H), 1.15 (d, J = 6.41 Hz, 3H), 0.88-0.77 (m, 1H), 0.75 (d, J = 6.24 Hz, 3H), 0.68 (d, J = 6.19 Hz, 3H). |
| 24 | 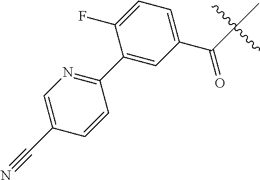 | (M + 2H)⁺² 637.3340, Calcd 637.3337. (M + 2Na)⁺² 659.8167, Calcd 659.8170. | 1H NMR (500 MHz, D₂O) δ 9.01 (dd, J = 0.81,2.07 Hz, 1H), 8.33 (dd, J = 2.13, 8.33 Hz, 1H), 8.27 (dd, J = 2.35, 7.14 Hz, 1H), 8.06-7.94 (m, 2H), 7.44 (dd, J = 8.78, 10.51 Hz, 1H), 7.37 (t, J = 7.22 Hz, 2H), 7.32 (t, J = 7.28 Hz, 1H), 7.24 (d, J = 7.06 Hz, 2H), 4.84-4.78 (m, 2H), 4.56 (t, J = 8.25 Hz, 1H), 4.51-4.43 (m, 2H), 4.37-4.12 (m, 7H), 3.51 (dd, J = 4.96, 13.46 Hz, 1H), 3.39- 3.26 (m, 2H), 3.24-2.97 (m, 9H), 2.93-2.82 (m, 1H), 2.82-2.72 (m, 1H), 2.41-2.30 (m, 1H), 2.30-2.07 (m, 5H), 2.07-1.79 (m, 4H), 1.52-1.43 (m, 1H), 1.43-1.34 (m, 1H), 1.21 (d, J = 6.41 Hz, 3H), 1.16 (d, J = 6.41 Hz, 3H), 0.87- 0.77 (m, 1), 0.75 (d, J = 6.31 Hz, 3H), 0.67 (d, J = 6.22 Hz, 3H). |
| 25 | 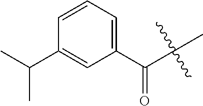 | (M + 2H)⁺² 598.3516, Calcd 598.3509. (M + Na)⁺ 1217.6771, Calcd 1217.6765. | 1H NMR (500 MHz, D₂O) δ 7.70 (s, 1H), 7.62 (d, J = 7.77 Hz, 1H), 7.57 (d, J = 7.79 Hz, 1H), 7.47 (t, J = .70 Hz, 1H), 7.38 (t, J = 7.26 Hz, 2H), 7.33 (t, J = 7.31 Hz, 1H), 7.25 (d, J = 7.12 Hz, 2H), 4.85-4.78 (m, 2H), 4.56 (t, J = 8.25 Hz, 1H), 4.51-4.42 (m, 2H), 4.38-4.12 (m, 7H), 3.51 (dd, J = 4.94, 13.44 Hz, 1H), 3.38-3.26 (m, 2H), 3.23-2.93 (m, 10H), 2.92-2.82 (m, 1H), 2.83-2.72 (m, 1H), 2.40-2.29 (m, 1H), 2.29- 2.07 (m, 5H), 2.07-1.79 (m, 4H), 1.53-1.43 (m, 1H), 1.43-1.34 (m, 1H), 1.24 (d, J = 6.95 Hz, 6H), 1.20 (d, J = 6.40 Hz, 3H), 1.16 (d, J = 6.38 Hz, 3H), 0.88-0.78 (m, 1H), 0.75 (d, J = 6.32 Hz, 3H), 0.68 (d, J = 6.22 Hz, 3H). |
| 26 | 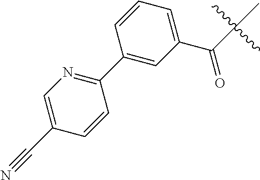 | (M + 2H)⁺² 628.3386, Calcd 628.3384. (M + Na)⁺ 1277.6495, Calcd 1277.6514. | 1H NMR (500 MHz, D₂O) δ 8.96 (d, J = 1.32 Hz, 1H), 8.37 (s, 1H), 8.29 (dd, J = 2.09, 8.36 Hz, 1H), 8.17 (d, J = 7.92 Hz, 1H), 8.04 (d, J = 8.36 Hz, 1H), 7.96 (d, J = 7.99 Hz, 1H), 7.70 (t, J = 7.83 Hz, 1H), 7.37 (t, J = 7.23 Hz, 2H), 7.32 (t, J = 7.26 Hz, 1H), 7.23 (d, J = 7.13 Hz, 2H), 4.85- 4.77 (m, 2H), 4.55 (t, J = 8.24 Hz, 1H), 4.49- 4.43 (m, 2H), 4.38-4.11 (m, 7H), 3.52 (dd, J = 4.095, 13.45 Hz, 1H), 3.39-3.26 (m, 2H), 3.25- 2.98 (m, 9H), 2.92-2.83 (m, 1H), 2.82-2.72 (m, 1H), 2.42-2.31 (m, 1H), 2.32-2.08 (m, 5H), 2.07-1.78 (m, 4H), 1.53-1.43 (m, 1H), 1.43-1.33 (m, 1H), 1.22 (d, J = 6.40 Hz, 3H), 1.15 (d, J = 6.39 Hz, 3H), 0.88-0.76 (m, 1H), 0.74 (d, J = 6.22 Hz, 3H), 0.67 (d, J = 6.16 Hz, 3H). |
| 27 | 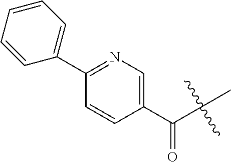 | (M + 2H)⁺² 615.8411, Calcd 615.8407. (M + Na)⁺ 1252.6555 Calcd 1252.6561. | 1H NMR (500 MHz, D₂O) δ 9.08 (d, J = 1.56 Hz, 1H), 8.61 (dd, J = 2.17, 8.46 Hz, 1H), 8.19 (d, J = 8.50 Hz, 1H), 7.94 (dd, J = 1.70, 7.84 Hz, 2H), 7.72-7.59 (m, 3H), 7.37 (t, J = 7.22 Hz, 2H), 7.32 (t, J = 7.29 Hz, 1H), 7.24 (d, J = 7.05 Hz, 2H), 4.87-4.76 (m, 2H), 4.55 (t, J = 8.27 Hz, 1H), 4.52-4.45 (m, 2H), 4.39-4.13 (m, 7H), 3.52 (dd, J = 4.98, 13.46 Hz, 1H), 3.38-3.27 (m, 2H), 3.27-2.98 (m, 9H), 2.94-2.82 (m, 1H), 2.83-2.72 (m, 1H), 2.42-2.32 (m, 1H), 2.32- 2.08 (m, 5H), 2.08-1.78 (m, 4H), 1.53-1.43 (m, 1H), 1.43-1.33 (m, 1H), 1.23 (d, J = 6.41 Hz, 3H), 1.16 (d, J = 6.41 Hz, 3H), 0.87-0.77 (m, 1H), 0.74 (d, J = 6.18 Hz, 3H), 0.67 (d, J = 6.14 Hz, 3H). |
| 28 | 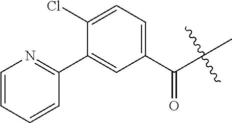 | (M + 2H)⁺² 632.8213, Calcd 632.8213. (M + 2Na)⁺² 655.3051, Calcd 655.3046. | 1H NMR (500 MHz, D₂O) δ 88.3 (d, J = 5.44 Hz, 1H), 8.58 (t, J = 7.89 Hz, 1H), 8.11 (d, J = 7.91 Hz, 1H), 8.07-7.96 (m, 3H), 7.81 (d, J = 8.30 Hz, 1H), 7.38 (t, J = 7.26 Hz, 2H), 7.32 (t, J = 7.28 Hz, 1H), 7.25 (d, J = 7.10 Hz, 2H), 4.86- 4.79 (m, 2H), 4.56 (t, J = 8.28 Hz, 1H), 4.52- 4.42 (m, 2H), 4.35-4.12 (m, 7H), 3.50 (dd, J = 4.95, 13.46 Hz, 1H), 3.39-3.26 (m, 2H), 3.24- 2.99 (m, 9H), 2.94-2.82 (m, 1H), 2.82-2.72 (m, 1H), 2.39-2.28 (m, 1H), 2.28-2.08 (m, 5H), 2.07-1.77 (m, 4H), 1.53-1.43 (m, 1H), 1.43-1.33 (m, 1H), 1.20 (d, J = 6.34 Hz, 3H), 1.17 (d, J = 6.40 Hz, 3H), 0.85-0.77 (m, 1H), 0.74 (d, J = 6.04 Hz, 3H), 0.67 (d, J = 6.08 Hz, 3H). |
| 29 | 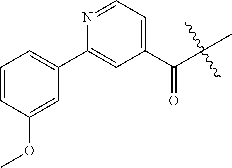 | (M + 2H)⁺² 630.8471, Calcd 630.8460. | 1H NMR (500 MHz, D₂O) δ 8.82 (d, J = 5.48 Hz, 1H), 8.35 (s, 1H), 7.97 (dd, J = 1.48, 5.62 Hz, 1H), 7.62-7.47 (m, 3H), 7.37 (t, J = .21 Hz, 2H), 7.32 (t, J = 7.22 Hz, 1H), 7.24 (d, J = 7.07 Hz, 3H), 4.86-4.77 (m, 2H), 4.55 (t, J = 8.26 Hz, 1H), 4.51-4.4 (m, 2), 4.37-4.12 (m, 7H), 3.92 (s, 3H), 3.51 (dd, J = 4.95, 13.47 Hz, 1H), 3.37-3.26 (m, 2H), 3.24-2.99 (m, 9H), 2.93- 2.82 (m, 1H), 2.82-2.72 (m, 1H), 2.41-2.31 (m, 1H), 2.31-2.08 (m, 5H), 2.07-1.77 (m, 4H), 1.53-1.43 (m, 1H), 1.43-1.33 (m, 1H), 1.22 (d, J = 6.38 Hz, 3H), 1.15 (d, J = 6.41 Hz, 3H), 0.87-0.77 (m, 1H), 0.75 (d, J = 6.26 Hz, 3H), 0.67 (d, J = 6.20 Hz, 3H). |
| 30 | 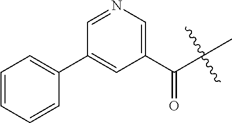 | (M + 2H)⁺² 615.8405, Calcd 615.8047. (M + Na)⁺ 12526556, Calcd 1252.6561. | 1H NMR (500 MHz, D₂O) δ 9.12 (d, J = 2.00 Hz, 1H), 9.04 (d, J = 1.84 Hz, 1H), 8.89-8.81 (m, 1H), 7.78 (d, J = 6.82 Hz, 2H), 7.66-7.52 (m, 3H), 7.37 (t, J = 7.24 Hz, 2H), 7.32 (t, J = 7.29 Hz, 1H), 7.24 (d, J = 7.09 Hz, 2H), 4.85-4.77 (m, 2H), 4.55 (t, J = 8.26 Hz, 1H), 4.52-4.45 (m, 2H), 4.37-4.13 (m, 7H), 3.51 (dd, J = 4.98, 13.45 Hz, 1H), 3.38-3.26 (m, 2H), 3.26-2.98 (m, 9H), 2.92-2.82 (m, 1H), 2.82-2.72 (m, 1H), 2.42-2.08 (s, 6H), 2.07-1.79 (m, 4H), 1.53-1.43 (m, 1H), 1.43-1.34 (m, 1H), 1.22 (d, J = 6.40 Hz, 3H), 1.16 (d, J = 6.41 Hz, 3H), 0.85- 0.77 (m, 1H), 0.75 (d, J = 6.17 Hz, 3H), 0.67 (d, J = 6.13 Hz, 3H). |
| 31 | 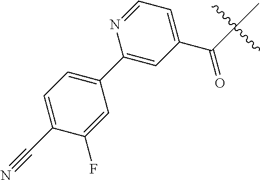 | (M + 2H)⁺² 637.3340, Calcd 637.3337. (M + 2Na)⁺² 659.3171, Calcd 649.3156. | 1H NMR (500 MHz, D₂O) δ 8.85 (dd, J = 0.60, 5.34 Hz, 1H), 8.28 (d, J = 0.69 Hz, 1H), 7.96- 7.90 (m, 2H), 7.87 (d, J = 9.15 Hz, 2H), 7.37 (t, J = 7.19 Hz, 2H), 7.3`1 (t, J = 7.28 Hz, 1H), 7.23 (d, J = 7.01 Hz, 2H), 4.84-4.78 (m, 2H), 4.55 (t, J = 8.25 Hz, 1H), 4.51-4.45 (m, 2H), 4.36-4.10 (m, 7H), 3.51 (dd, J = 5.00, 13.44 Hz, 1H), 3.36- 3.26 (m, 2H), 3.23-2.98 (m, 9H), 2.93-2.82 (m, 1H), 2.82-2.73 (m, 1H), 2.41-2.31 (m, 1H), 2.31-2.07 (m, 5H), 2.07-1.79 (m, 4H), 1.52-1.42 (m, 1H), 1.42-1.32 (m, 1H), 1.22 (d, J = 6.41 Hz, 3H), 1.15 (d, J = 6.42 Hz, 3H), 0.85- 0.76 (m, 1H), 0.74 (d, J = 6.07 Hz, 3H), 0.67 (d, J = 6.07 Hz, 3H). |
| 32 | 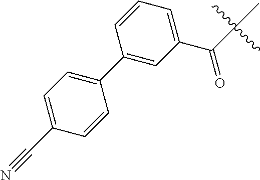 | (M + 2H)⁺² 627.8407, Calcd 627.8407. (M + Na)⁺ 1276.6559, Calcd 1276.6561. | 1H NMR (500 MHz, D₂O) δ 8.09-8.06 (m, 1H), 7.95-7.91 (m, 1H), 7.89-7.80 (m, 5H), 7.65 (t, J = 7.82 Hz, 1H), 7.37 (t, J = 7.21 Hz, 2H), 7.32 (t, J = 7.27 Hz, 1H), 7.23 (d, J = 7.04 Hz, 2H), 4.83-4.77 (m, 2H), 4.55 (t, J = 8.25 Hz, 1H), 4.51-4.43 (m, 2H), 4.38-4.30 (m, 1H), 4.29- 4.16 (m, 5H), 4.14 (d, J = 4.55 Hz, 1H), 3.52 (dd, J = 4.98, 13.44 Hz, 1H), 3.36-3.25 (m, 2H), 3.23-2.97 (m, 9H), 2.92-2.81 (m, 1H), 2.81- 2.72 (m, 1H), 2.41-2.31 (m, 1H), 2.31-2.07 (m, 5H), 2.07-1.78 (m, 4H), 1.52-1.42 (m, 1H), 1.42-1.33 (m, 1H), 1.21 (d, J = 6.41 Hz, 3H), 1.14 (d, J = 6.41 Hz, 3H), 0.88-0.77 (m, 1H), 0.75 (d, J = 6.34 Hz, 3H), 0.67 (d, J = 6.24 Hz, 3H). |
| 33 | 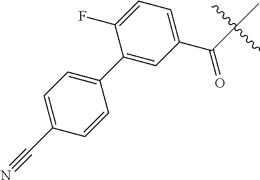 | (M + 2H)⁺² 636.8370, Calcd 636.8360. (M + Na)⁺ 1294.6484, Calcd 1294.6467. | 1H NMR (500 MHz, D₂O) δ 7.97 (dd, J = 2.13, 7.22 Hz, 1H), 7.92-7.83 (m, 3H), 7.76 (d, J = 7.80 Hz, 2H), 7.42-7.27 (m, 4H), 7.23 (d, J = 7.15 Hz, 2H), 4.84-4.77 (m, 2H), 4.55 (t, J = 8.24 Hz, 1H), 4.50-4.42 (m, 2H), 4.35-4.11 (m, 7H), 3.51 (dd, J = 4.90, 13.48 Hz, 1H), 3.37- 3.25 (m, 2H), 3.25-2.97 (m, 9H), 2.93-2.82 (m, 1H), 2.82-2.72 (m, 1H), 2.40-2.29 (m, 1H), 2.29-2.08 (m, 5H), 2.07-1.77 (m, 4H), 1.52-1.42 (m, 1H), 1.42-1.32 (m, 1H), 1.20 (d, J = 8.20 Hz, 3H), 1.15 (d, JU = 6.38 Hz, 3H), 0.89- 0.76 (m, 1H), 0.74 (d, J = 6.06 Hz, 3H), 0.67 (d, J = 6.05 Hz, 3H). |
| 34 | 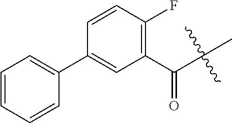 | (M + 2H)⁺² 624.3392, Calcd 624.3384. (M + 2Na)⁺² 1269.6516, Calcd 1269.6515 | 1H NMR (500 MHz, D₂O) δ 7.93 (dd, J = 2.33, 6.75 Hz, 1H), 7.90-7.84 (m, 1H), 7.68 (d, J = 7.45 Hz, 2H), 7.53 (t, J = 7.60 Hz, 2H), 7.45 (t, J = 7.38 Hz, 1H), 7.40-7.29 (m, 4H), 7.24 (d, J = 7.14 Hz, 2H), 4.83-4.76 (m, 2H), 4.55 (t, J = 8.22 Hz, 1H), 4.51-4.43 (m, 2H), 4.37-4.30 (m, 1H), 4.30-4.16 (m, 5H), 4.15 (d, J = 4.52 Hz, 1H), 3.52 (dd, J = 4.97, 13.47 Hz, 1H), 3.37- 3.25 (m, 2H), 3.25-2.98 (m, 9H), 2.92-2.82 (m, 1H), 2.81-2.71 (m, 1H), 2.41-2.30 (m, 1H), 2.30-2.06 (m, 5H), 2.06-1.79 (m, 4H), 1.52-1.43 (m, 1H), 1.423-1.33 (m, 1H), 1.23 (d, J = 6.38 Hz, 3H), 1.14 (d, J = 6.39 Hz, 3H), 0.87- 0.77 (m, 1H), 0.75 (d, J = 6.26 Hz, 3H), 0.67 (d, J = 6.18 Hz, 3H). |
| 35 | 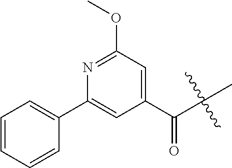 | (M + 2H)⁺² 630.8465, Calcd 630.8460. (M + Na)⁺ 1282.6654, Calcd 1282.6667. | 1H NMR (500 MHz, D₂O) δ 8.01 (dd, J = 1.51, 7.94 Hz, 2H), 7.75 (d, J = 0.97 Hz, 1H), 7.59- 7.50 (m, 3H), 7.37 (t, J = 7.24 Hz, 2H), 7.32 (t, J = 7.26 Hz, 1H), 7.24 (d, J = 7.10 Hz, 2H), 7.14 (d, J = 0.97 Hz, 1H), 4.83-4.77 (m, 2H), 4.55 (t, J = 8.24 Hz, 1H), 4.50-4.43 (m, 2H), 4.37-4.29 (m, 1H), 4.29-4.16 (m, 5H), 4.14 (d, J = 4.53 Hz, 1H), 4.05 (s, 3H), 3.52 (dd, J = 5.01, 13.44 Hz, 1H), 3.38-3.25 (m, 2H), 3.23-2.99 (m, 9H), 2.91-2.82 (m, 1H), 2.81-2.72 (m, 1H), 2.41-2.30 (m, 1H), 2.30-2.06 (m, 5H), 2.05- 1.78 (m, 4H), 1.52-1.42 (m, 1H), 1.42-1.33 (m, 1H), 1.22 (d, J = 6.40 Hz, 3H), 1.14 (d, J = 6.41 Hz, 3H), 0.86-0.77 (m, 1H), 0.74 (d, J = 6.31 Hz, 3H), 0.67 (d, J = 6.21 Hz, 3H). |
| 36 | 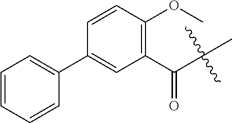 | (M + 2H)⁺² 630.3481, Calcd 630.3484. (M + 2Na⁾⁺² 652.3315, Calcd 652.3303. | 1H NMR (500 MHz, D₂O) δ 8.02 (d, J = 2.39 Hz, 1H), 7.87 (dd, J = 2.42, 8.71 Hz, 1H), 7.68 (d, J = 7.43 Hz, 2H), 7.52 (t, J = 7.71 Hz, 2H), 7.45- 7.27 (m, 5H), 7.24 (d, J = 7.15 Hz, 2H), 4.81- 4.75 (m, 2H), 4.56 (t, J = 8.24 Hz, 1H), 4.51- 4.44 (m, 2H), 4.38-4.11 (m, 7H), 3.99 (s, 3H), 3.53 (dd, J = 5.07, 13.46 Hz, 1H), 3.38-3.24 (m, 2H), 3.23-2.96 (m, 9H), 2.90-2.81 (m, 1H), 2.81-2.71 (m, 1H), 2.42-2.31 (m, 1H), 2.29- 2.05 (m, 5H), 2.05-1.79 (m, 4H), 1.53-1.44 (m, 1H), 1.43-1.34 (m, 1H), 1.23 (d, J = 6.39 Hz, 3H), 1.13 (d, J = 6.40 Hz, 3H), 0.87-0.77 (m, 1H), 0.75 (d, J = 6.21 Hz, 3H), 0.68 (d, J = 6.13 Hz, 3H). |
| 37 | 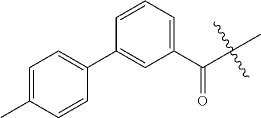 | (M + 2H)⁺² 622.3506, Calcd 622.3509. | 1H NMR (500 MHz, D₂O) δ 8.05 (s, 1H), 7.91 (d, J = 7.58 Hz, 1H), 7.78 (d, J = 7.77 Hz, 1H), 7.66- 7.59 (m, 3H), 7.37 (t, J = 8.19 Hz, 4H), 7.32 (t, J = 7.28 Hz, 1H), 7.24 (d, J = 7.06 Hz, 2H), 4.83- 4.76 (m, 2H), 4.57 (t, J = 8.24 Hz, 1H), 4.50- 4.41 (m, 2H), 4.37-4.29 (m, 1H), 4.29-4.16 (m, 5H), 4.14 (d, J = 4.52 Hz, 1H), 3.51 (dd, J = 5.01, 13.45 Hz, 1H), 3.37-3.24 (m, 2H), 3.24- 2.98 (m, 9H), 2.89-2.80 (m, 1H), 2.79-2.70 (m, 1H), 2.42-2.31 (m, 4H), 2.31-2.06 (m, 5H), 2.05-1.76 (m, 4H), 1.52-1.43 (m, 1H), 1.43-1.35 (m, 1H), 1.21 (d, J = 6.39 Hz, 3H), 1.13 (d, J = 6.37 Hz, 3H), 0.93-0.79 (m, 1H), 0.75 (d, J = 6.47 Hz, 3H), 0.68 (d, J = 6.37 Hz, 3H). |
| 38 | 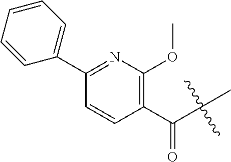 | (M + 2H)⁺² 630.8463, Calcd 630.8460. (M + Na)⁺ 1282.6665, Calcd 1282.6667. | 1H NMR (500 MHz, D₂O) δ 8.25 (d, J = 7.92 Hz, 1H), 8.13-8.07 (m, 2H), 7.61 (d, J = 7.95 Hz, 1H), 7.57-7.50 (m, 3H), 7.37 (t, J = 7.24 Hz, 2H), 7.32 (t, J = 7.29 Hz, 1H), 7.24 (d, J = 7.07 Hz, 2H), 4.83-4.76 (m, 2H), 4.55 (t, J = 8.25 Hz, 1H), 4.51-4.44 (m, 2H), 4.37-4.09 (m, 10H), 3.53 (dd, J = 5.03, 13.42 Hz, 1H), 3.38-3.26 (m, 2H), 3.22-2.96 (m, 9H), 2.93-2.82 (m, 1H), 2.82-2.72 (m, 1H), 2.43-2.31 (m, 1H), 2.30- 2.06 (m, 5H), 2.06-1.79 (m, 4H), 1.52-1.42 (m, 1H), 1.42-1.33 (m, 1H), 1.23 (d, J = 6.42 Hz, 3H), 1.14 (d, J = 6.41 Hz, 3H), 0.85-0l.76 (m, 1H), 0.74 (d, J = 6.24 Hz, 3H), 0.67 (d, J = 6.15 Hz, 3H). |
| 39 | 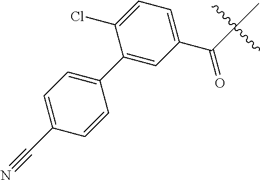 | (M + 2H)⁺² 644.8216, Calcd 644.8213. | 1H NMR (500 MHz, D₂O) δ 7.87 (d, J = 8.40 Hz, 2H), 7.84-7.79 (m, 2H), 7.70 (d, J = 8.94 Hz, 1H), 7.67 (d, J = 8.40 Hz, 2H), 7.37 (t, J = 7.25 Hz, 2H), 7.32 (t, J = 7.27 Hz, 1H), 7.24 (d, J = 7.09 Hz, 2H), 4.83-4.76 (m, 2H), 4.56 (t, J = 8.24 Hz, 1H), 4.50-4.41 (m, 2H), 4.36-4.17 (m, 6H), 4.15 (d, J = 4.55 Hz, 1H), 3.51 (dd, J = 4.96, 13.44 Hz, 1H), 3.36-3.24 (m, 2H), 3.23- 2.99 (m, 9H), 2.92-2.82 (m, 1H), 2.82-2.72 (m, 1H), 2.39-2.28 (m, 1H), 2.28-2.08 (m, 5H), 2.06-1.76 (m, 4H), 1.51-1.42 (m, 1H), 1.43-1.33 (m, 1H), 1.21-1.17 (m, 3H), 1.15 (d, J = 6.41 Hz, 3H), 0.91-0.77 (m, 1H), 0.75 (d, J = 6.33 Hz, 3H), 0.67 (d, J = 6.24 Hz, 3H). |
| 40 | 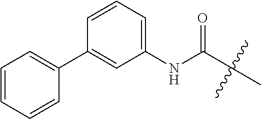 | (M + 2H)⁺² 622.8486, Calcd 622.8486. (M + 2Na⁾⁺² 644.8296, Calcd 644.8305. | ¹H NMR (500 MHz, D₂O) δ 7.66-7.60 (m, 2H), 7.57 (s, 1H), 7.47 (t, J = 7.61 Hz, 2H), 7.44- 7.36 (m, 3H), 7.32 (t, J = 7.22 Hz, 2H), 7.29- 7.24 (m, 2H), 7.19 (d, J = 7.10 Hz, 2H), 4.73- 4.71 (m, 1H), 4.50 (t, J = 8.24 Hz, 1H), 4.44- 4.36 (m, 3H), 4.32-4.25 (m, 1H), 4.24-4.11 (m, 5H), 4.10 (d, J = 4.60 Hz, 1H), 3.39 (dd, J = 4.97, 13.43 Hz, 1H), 3.30-3.16 (m, 2H), 3.16- 2.94 (m, 9H), 2.85-2.75 (m, 1H), 2.74-2.64 (m, 1H), 2.28-1.99 (m, 6H), 1.99-1.71 (m, 4H), 1.47-1.37 (m, 1), 1.38-1.28 (m, 1H), 1.17 (d, J = 6.41 Hz, 3H), 1.08 (d, J = 6.38 Hz, 3H), 0.84-0.72 (m, 1H), 0.70 (d, J = 6.38 Hz, 3H), 0.63 (d, J = 6.26 Hz, 3H). |
| 41 | 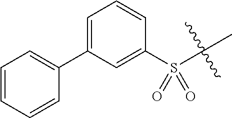 | (M + 2H)⁺² 633.3266, Calcd 633.3266. (M + Na)⁺ 1287.6269, Calcd 1287.6279. | 1H NMR (500 MHz, D₂O) δ 8.12-8.09 (m, 1H), 8.03-7.98 (m, 1H), 7.88-7.83 (m, 1H), 7.77- 7.68 (m, 3H), 7.56 (t, J = 7.53 Hz, 2H), 7.49 (t, J = 7.37 Hz, 1H), 7.37 (t, J = 7.24 Hz, 2H), 7.32 (t, J = 7.28 Hz, 1H), 7.24 (d, J = 7.06 Hz, 2H), 4.74- 4.70 (m, 1H), 4.55 (t, J = 8.26 Hz, 1H), 4.47 (dd, J = 5.46, 8.89 Hz, 1H), 4.31-4.13 (m, 6H), 4.10 (dd, J = 4.66, 9.67 Hz, 1H), 4.03 (d, J = 3.65 Hz, 1H), 3.94-3.86 (m, 1H), 3.47 (dd, J = 4.95, 13.43 Hz, 1H), 3.33-3.19 (m, 2H), 3.18-2.96 (m, 9H), 2.91-2.80 (m, 1H), 2.79-2.69 (m, 1H), 2.30-2.05 (m, 5H), 2.04-1.75 (m, 5H), 1.53-1.43 (m, 1H), 1.43-1.34 (m, 1H), 1.16 (d, J = 6.41 Hz, 3H), 0.85 (d, J = 6.42 Hz, 3H), 0.83- 0.77 ( m, 1H), 0.75 (d, J = 6.32 Hz, 3H), 0.68 (d, J = 6.22 Hz, 3H). |
| 42 | 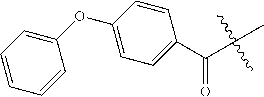 | (M + 2H)⁺² 623.3412, Calcd 623.3406. | ¹H NMR (500 MHz, D₂O) δ 7.76 (d, J = 8.86 Hz, 2H), 7.44-7.38 (m, 2H), 7.32 (t, J = 7.23 Hz, 2H), 7.29-7.16 (m, 4H), 7.12-7.07 (m, 2H), 7.05 (d, J = 8.86 Hz, 2H), 4.76-4.62 (m, 2H), 4.50 (t, J = 8.25 Hz, 1H), 4.44-4.37 (m, 2H), 4.31-4.08 (m, 7H), 3.44 (dd, J = 4.95, 13.43 Hz, 1H), 3.30-3.21 (m, 2H), 3.14-2.96 (m, 9H), 2.87-2.77 (m, 1H), 2.76-2.66 (m, 1H), 2.33- 2.22 (m, 1H), 2.22-2.01 (m, 5H), 2.01-1.72 (m, 4H), 1.47-1.37 (m, 1H), 1.37-1.28 (m, 1H), 1.14 (d, J = 6.41 Hz, 3H), 1.10 (d, J = 6.40 Hz, 3H), 0.83-0.71 (m, 1H), 0.69 (d, J = 6.33 Hz, 3H), 0.62 (d, J = 6.24 Hz, 3H). |
| 43 | 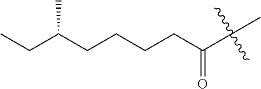 | (M + 2H)⁺² 595.3749, Calcd¹ 595.3744. (M + 2Na)⁺² 617.3563, Calcd 617.3564. | ¹H NMR (500 MHz, D₂O) δ 7.33 (t, J = 7.23 Hz, 2H), 7.28 (t, J = 7.27 Hz, 1H), 7.20 (d, J = 7.09 Hz, 2H), 4.79-4.73 (m, 1H), 4.51 (t, J = 8.24 Hz, 1H), 4.48-4.41 (m, 2H), 4.36 (d, J = 3.81 Hz, 1H), 4.28-4.11 (m, 7H), 3.45 (dd, J = 4.97, 13.49 Hz, 1H), 3.33-3.22 (m, 2H), 3.13-2.96 (m, 9H), 2.86-2.77 (m, 1H), 2.7-2.67 (m, 1H), 2.27 (t, J = 7.35 Hz, 2H), 2.23-1.75 (m, 10H), 1.59-1.47 (m, 2H), 1.47-1.39 (m, 1H), 1.38-1.29 (m, 1H), 1.30-1.17 (m, 5H), 1.17- 1.10 (m, 6H), 1.10-0.99 (m, 2H), 0.80-0.73 (m, 7H), 0.70 (d, J = 6.37 Hz, 3H), 0.63 (d, J = 6.27 Hz, 3H). |
| 44 | 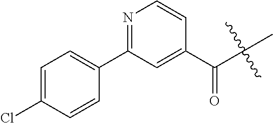 | (M + 2H)⁺² 632.8212. Calcd¹ 632.8213. | ¹H NMR (500 MHz, D₂O) δ 8.80 (d, J = 5.52 Hz, 1H), 8.28 (s, 1H), 7.93-7.84 (m, 3H), 7.62 (d, J = 8.55 Hz, 2H), 7.42-7.28 (m, 3H), 7.24 (d, J = 7.15 Hz, 2H), 4.86-4.77 (m, 2H), 4.55 (t, J = 8.23 Hz, 1H), 4.51-4.44 (m, 2H), 4.37-4.11 (m, 7H), 3.51 (dd, J = 4.97, 13.43 Hz, 1H), 3.38- 3.26 (m, 2H), 3.25-2.99 (m, 9H), 2.93-2.81 (m, 1H), 2.81-2.71 (m, 1H), 2.42-2.31 (m, 1H), 2.31-2.07 (m, 5H), 2.06-1.78 (m, 4H), 1.53-1.43 (m, 1H), 1.42-1.34 (m, 1H), 1.22 (d, J = 6.40 Hz, 3H), 1.15 (d, J = 6.39 Hz, 3H), 0.88- 0.77 (m, 1H), 0.75 (d, J = 6.29 Hz, 3H), 0.67 (d, J = 6.20 Hz, 3H). |
| 45 | 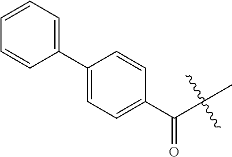 | (M + 2H)⁺² 615.343. Calcd¹ 615.3431. | ¹H NMR (500 MHz, D₂O) δ 7.89-7.83 (m, 2H), 7.80-7.75 (m, 2H), 7.73-7.67 (m, 2H), 7.53- 7.47 (m, 2H), 7.46-7.40 (m, 1H), 7.36-7.30 (m, 2H), 7.29-7.25 (m, 1H), 7.19 (d, J = 6.77 Hz, 2H), 4.78-4.73 (m, 2H), 4.51 (t, J = 8.22 Hz, 1H), 4.45-4.39 (m, 2H), 4.32-4.25 (m, 1H), 4.25-4.12 (m, 5H), 4.10 (d, J = 4.50 Hz, 1H), 3.47 (dd, J = 5.06, 13.48 Hz, 1H), 3.32-3.21 (m, 2H), 3.20-2.95 (m, 9H), 2.86-2.76 (m, 1H), 2.76-2.66 (m, 1H), 2.37-2.27 (m, 1H), 2.26- 2.01 (m, 5H), 2.01-1.74 (m, 4H), 1.47-1.38 (m, 1H), 1.38-1.29 (m, 1), 1.17 (d, J = 6.40 Hz, 3H), 1.09 (d, J = 6.35 Hz, 3H), 0.84-0.73 (m, 1H), 0.70 (d, J = 6.44 Hz, 3H), 0.63 (d, J = 6.30 Hz, 3H). |

¹Calcd means calculated.

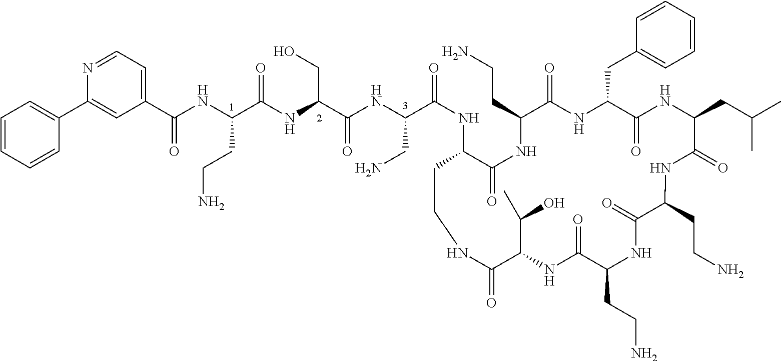

### Example 46

Example 46 was prepared using a similar procedure as described for Example 1 except that L-serine was used instead of L-threonine at the two (2) position. ¹H NMR (500 MHz, D₂O) δ 8.84 (d, J=5.69 Hz, 1H), 8.42 (d, J=0.86 Hz, 1H), 8.03 (dd, J=1.62, 5.74 Hz, 1H), 7.93 (dd, J=1.83, 7.66 Hz, 2H), 7.70-7.60 (m, 3H), 7.42-7.28 (m, 3H), 7.24 (d, J=7.10 Hz, 2H), 4.85-4.78 (m, 2H), 4.60-4.50 (m, 2H), 4.47 (dd, J=5.47, 8.89 Hz, 1H), 4.32-4.12 (m, 6H), 3.98 (dd, J=5.43, 11.37 Hz, 1H), 3.89 (dd, J=5.72, 11.38 Hz, 1H), 3.53 (dd, J=5.02, 13.43 Hz, 1H), 3.39-3.25 (m, 2H), 3.25-2.99 (m, 9H), 2.92-2.82 (m, 1H), 2.81-2.72 (m, 1H), 2.42-2.30 (m, 1H), 2.30-2.06 (m, 5H), 2.06-1.78 (m, 4H), 1.52-1.43 (m, 1H), 1.43-1.33 (m, 1H), 1.15 (d, J=6.39 Hz, 3H), 0.89-0.77 (m, 1H), 0.75 (d, J=6.26 Hz, 3H), 0.68 (d, J=6.18 Hz, 3H); HRMS: (M+2H)⁺² 608.8331; Calcd 608.8329.

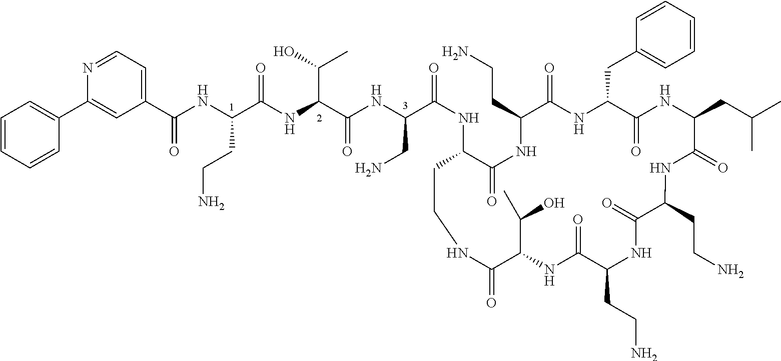

### Example 47

Example 47 was prepared using a similar procedure as described for Example 1 except that D-Dap was used instead of L-Dap at the three (3) position. ¹H NMR (500 MHz, D₂O) δ 8.78 (d, J=5.70 Hz, 1H), 8.40-8.29 (m, 1H), 7.96 (dd, J=1.64, 5.70 Hz, 1H), 7.92-7.81 (m, 2H), 7.65-7.54 (m, 3H), 7.33-7.21 (m, 3H), 7.18-7.10 (m, 2H), 4.79-4.72 (m, 2H), 4.50 (t, J=8.18 Hz, 1H), 4.41 (d, J=3.68 Hz, 1H), 4.36 (dd, J=5.51, 8.82 Hz, 1H), 4.34-4.27 (m, 1H), 4.24-4.11 (m, 5H), 4.08 (d, J=4.67 Hz, 1H), 3.49 (dd, J=5.59, 13.40 Hz, 1H), 3.33-3.21 (m, 2H), 3.21-3.11 (m, 2H), 3.11-2.90 (m, 7H), 2.86-2.75 (m, 1H), 2.75-2.64 (m, 1H), 2.39-2.27 (m, 1H), 2.27-2.00 (m, 5H), 2.00-1.74 (m, 4H), 1.47-1.37 (m, 1H), 1.37-1.28 (m, 1H), 1.18 (d, J=6.37 Hz, 3H), 1.09 (d, J=6.39 Hz, 3H), 0.83-0.72 (m, 1H), 0.69 (d, J=6.33 Hz, 3H), 0.62 (d, J=6.24 Hz, 3H). HRMS: (M+2H)⁺² 615.8408; Calcd 615.8407.

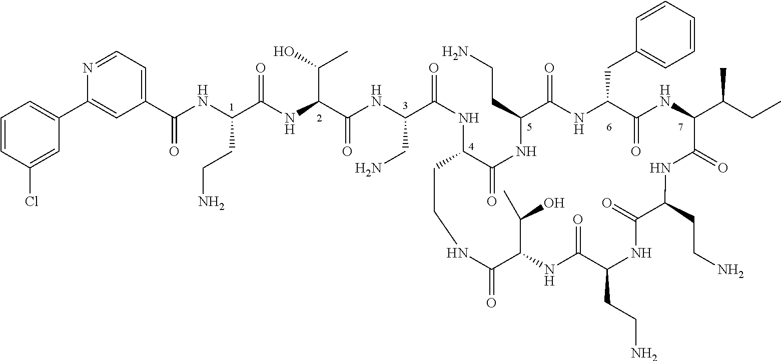

### Example 48

Example 48 was prepared using a similar procedure as described for Example 3 except that L-isoleucine was used instead of L-leucine at the seven (7) position. ¹H NMR (500 MHz, D₂O) δ 8.73 (dd, J=0.88, 5.30 Hz, 1H), 8.13 (dd, J=0.86, 1.55 Hz, 1H), 7.93-7.87 (m, 1H), 7.82-7.76 (m, 1H), 7.74 (dd, J=1.63, 5.33 Hz, 1H), 7.55-7.46 (m, 2H), 7.35-7.28 (m, 2H), 7.28-7.22 (m, 1H), 7.19 (d, J=6.81 Hz, 2H), 4.77-4.63 (m, 3H), 4.43 (d, J=3.90 Hz, 1H), 4.38-4.34 (m, 1H), 4.30-4.22 (m, 2H), 4.22-4.06 (m, 5H), 3.46 (dd, J=5.04, 13.44 Hz, 1H), 3.32-2.89 (m, 11H), 2.78-2.68 (m, 1H), 2.68-2.58 (m, 1H), 2.38-2.00 (m, 6H), 1.94-1.69 (m, 5H), 1.19-1.13 (m, 4H), 1.09 (d, J=6.18 Hz, 3H), 0.97-0.84 (m, 1H), 0.76-0.69 (m, 3H), 0.65 (d, J=6.83 Hz, 3H). HRMS: (M+2H)⁺² 632.8212; Calcd 632.8213.

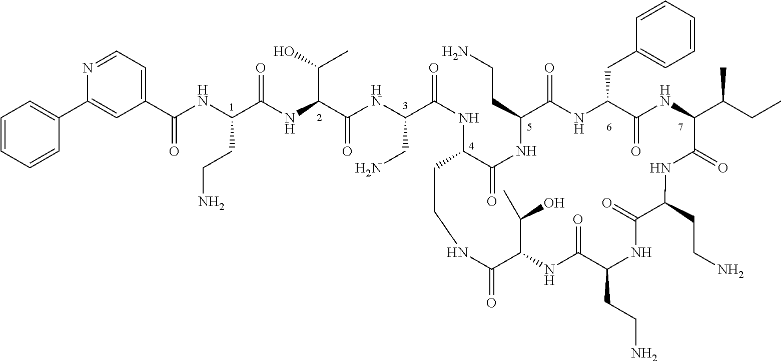

### Example 49

Example 49 was prepared using a similar procedure as described for Example 1 except that L-isoleucine was used instead of L-leucine at the seven (7) position. ¹H NMR (500 MHz, D₂O) δ 8.81-8.74 (m, 1H), 8.34-8.26 (m, 1H), 7.96-7.83 (m, 3H), 7.66-7.54 (m, 3H), 7.36-7.28 (m, 2H), 7.28-7.23 (m, 1H), 7.20 (d, J=6.78 Hz, 2H), 4.80-4.63 (m, 3H), 4.43 (d, J=3.91 Hz, 1H), 4.40-4.34 (m, 1H), 4.30-4.23 (m, 2H), 4.23-4.07 (m, 5H), 3.46 (dd, J=4.99, 13.39 Hz, 1H), 3.31-2.89 (m, 11H), 2.77-2.68 (m, 1H), 2.68-2.59 (m, 1H), 2.37-1.99 (m, 6H), 1.94-1.70 (m, 5H), 1.19-1.13 (m, 4H), 1.10 (d, J=6.15 Hz, 3H), 0.97-0.85 (m, 1H), 0.76-0.69 (m, 3H), 0.65 (d, J=6.82 Hz, 3H). HRMS: (M+2H)⁺² 615.8411; Calcd 615.8407.

Alternatively, the compounds of the present invention may be prepared using the synthetic methodology described in Scheme 1 and Example 50.

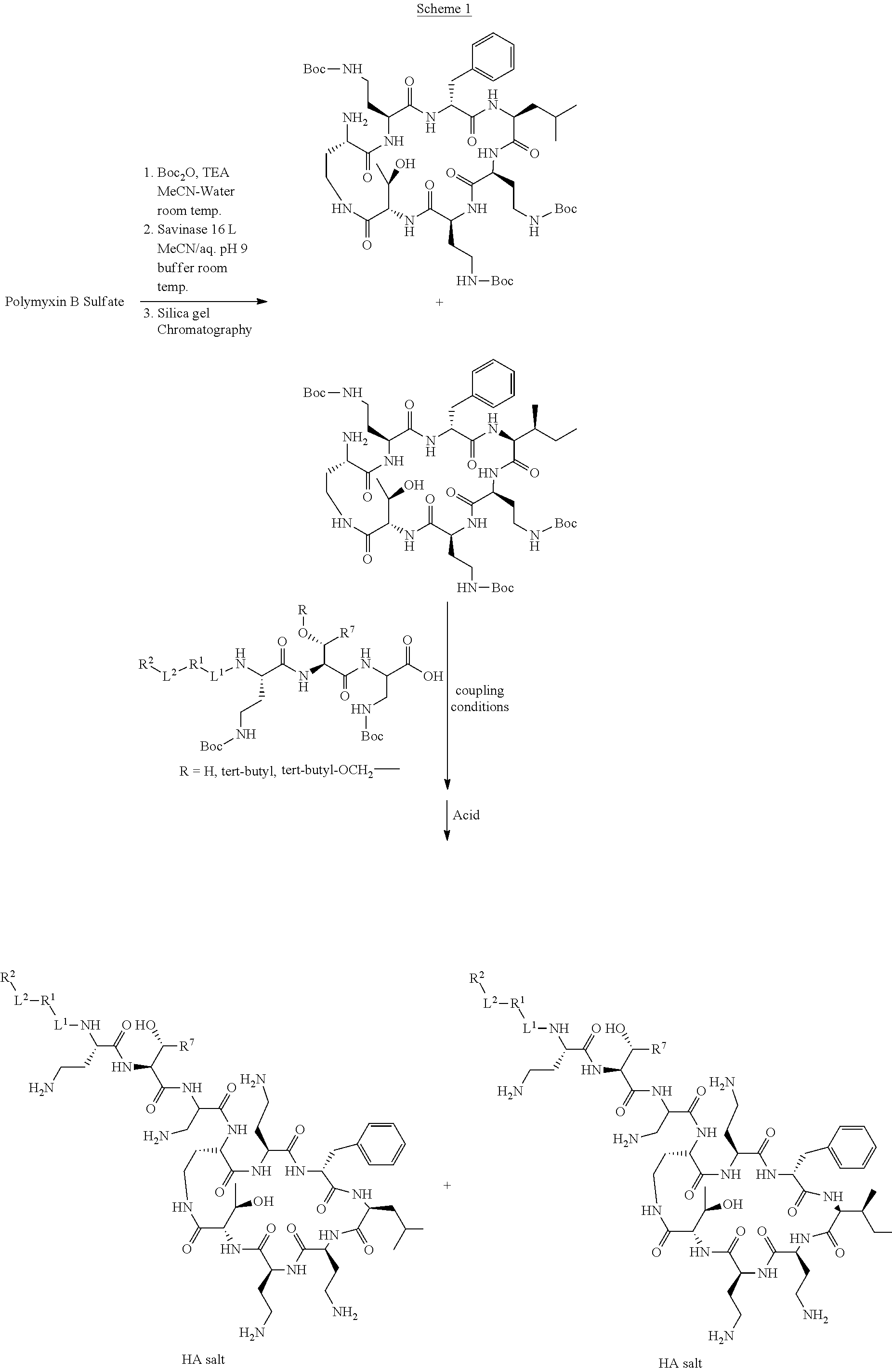

Commercially purchased polymyxin B sulfate is treated with Boc-anhydride or Boc-ON and then with savinase. Savinase enzymatically cleaves the fatty acid-tripeptide “tail” from the cyclic heptapeptide to provide the tri-protected cyclic heptapeptide containing both L-Leu and L-Ile. The ratio of L-Leu to L-Ile is dependent on the ratio found in the purchased product, polymyxin B sulfate. However, the L-Leu:L-Ile ratio of the final products may vary from the purchased ratio (polymyxin B sulfate ratio) depending on the inherent reactivity of each L-Leu derivative and L-Ile derivative and on the purification techniques used during the total synthesis of the final products. The cyclic heptapeptide is then coupled to a di-protected three amino acid derivative using standard coupling techniques well known to those in the art. For example, the coupling may proceed using a carbodiimide (EDC-HCl, DIC, or DCC) and HOBt to provide the desired decapeptide. The decapeptide is then subjected to acidic conditions to remove the amino and hydroxy protecting groups and give the compound of Formula II or III as a salt. Acids that may be used to remove the protecting groups include, but are not limited to, trifluoroacetic and HCl. The salt may be exchanged to p-toluenesulfonate, methanesulfonate, or sulfate (or HCl when TFA is used to deprotect) using standard techniques well known to those in the art.

Polymyxin B sulfate, purchased commercially, is a fermentation product. Fermentation residuals and byproducts are in general different from impurities found in chemical synthesis. Inherent in biological fermentation processes is greater variability in composition of product than obtained via direct chemical synthesis. Thus, those skilled in the art recognize that fermentation processes are subject to variability in fermentation products. Such variability is expected to result in observed differences in fermentation products between different fermentation lots.

The synthetic methodology described by Scheme 1 provides that the cyclic heptapeptide is separated from the fatty acid-tripeptide “tail.” Therefore, it is to be understood that the present invention encompasses any variability found in the cyclic heptapeptide that originated from the purchased polymyxin B sulfate. For example, the percentage of isoleucine found in purchased polymyxin B sulfate at position seven (7) may vary up to 20% (ratio range of 100:0 to 80:20 of L-Leu to L-Ile). More typically, the observed percentage is 7% to 15% of isoleucine or a ratio range of 93:7 to 85:15 of L-Leu to L-Ile.

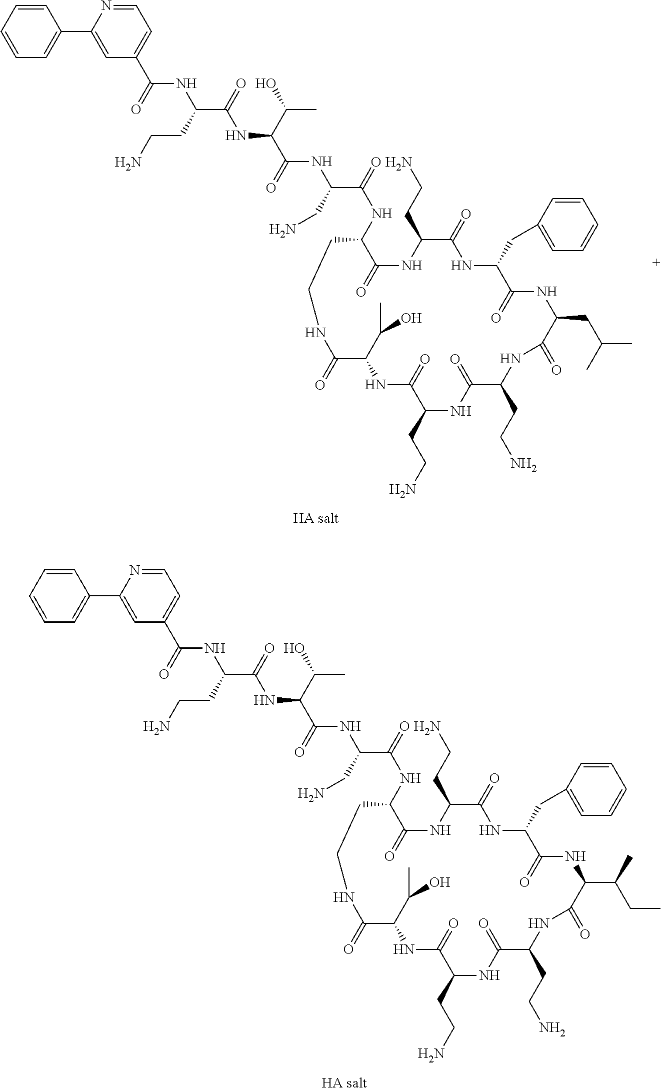

### Example 50

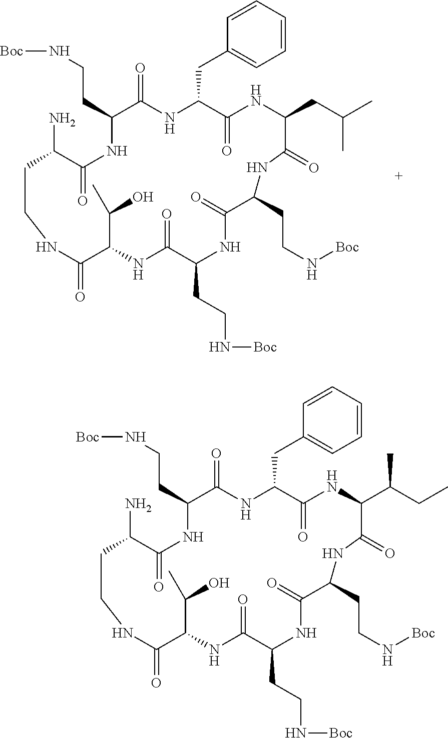

### Example 50A

To a single neck round bottom flask at ambient temp (18° C.) was charged Polymyxin B Sulfate (50 g; 1.00 equiv; 38.42 mmoles), purchased from Xellia Pharmaceuticals AS, and acetonitrile (500.00 mL) and water (250.00 mL) were added. The mixture was stirred at ambient temp for 5 min. Triethylamine (6 equiv; 230.49 mmoles; 32.13 mL) was added. After 10 min, the mixture turned to a white slurry. Di-t-butyldicarbonate (50.81 g; 6 equiv; 230.49 mmoles;) was added in one portion. The reaction was stirred at ambient temperature, and turned clear in 1 h. Sampling of the reaction mixture by LCMS indicated complete conversion of the starting material. The total volume of the reaction mixture was ˜850 mL. To the mixture was charged 150 mL of an enzyme solution (Savinase 16L Type EX, declared activity 16 KNPU-s/g; purchased from Novazymes). The pH of the resulting mixture was adjusted to 9.0 with 5 ml 4N NaOH. The mixture was stirred at room temperature with magnetic stirring. Additional 50 ml of enzyme solution was added after 17 h, and another 50 ml of enzyme solution added after 43 h. After the overall reaction time of 65 h, the reaction was quenched by adding 1 L of ethyl acetate. The layers were separated, and the organic phase was concentrated to dryness to give a light yellow oil (137.8 g, containing some propylene glycol that was used a stabilizer of the enzyme and the sidechain cleaved from Polymyxin B). The material was loaded onto flash silica gel (silica gel/crude product; 30:1 ratio), and the silica gel column was eluted with DCM/MeOH/TEA (93/6/1). Pure fractions containing the tri-Boc protected heptapeptide were combined and concentrated to dryness to give the desired product as a white solid (24.5 g) in a ratio of L-Leu to L-Ile of 93:7 to 91:9.

### Example 50B

Under N₂ atmosphere, to a 500 mL round bottom flask is added the ph-pyr-C(O)-(L-Dab)-(L-Thr)-(L-Dap-OH) side chain (12.9 g, 18.8 mmol). DMF (65 mL) is added, followed by TBTU (O-(1,2-Dihydro-2-oxo-1-pyridyl)-N,N,N′,N′-tetramethyluroniumTetrafluoroborate; 5.9 g, 19.8 mmol). The mixture is stirred at ambient temperature for 2 h. The tri-Boc protected heptapeptide from Example 50A (20.0 g, 18.8 mmol) is added to the reaction pot as a solution in ethyl acetate (100 mL) and stirred until the reaction is complete by HPLC analysis (2-16 h at ambient temperature). Ethyl Acetate (200 mL) is added, the mixture is stirred, and the phases allowed to settle. The layers are separated and the aqueous phase is extracted with ethyl acetate (1×100 mL). The organic layers are combined, washed with 10% aqueous NaHCO3 solution and brine successively, and dried over anhydrous magnesium sulfate. The resulting organic solution is concentrated to dryness to give the coupling product.

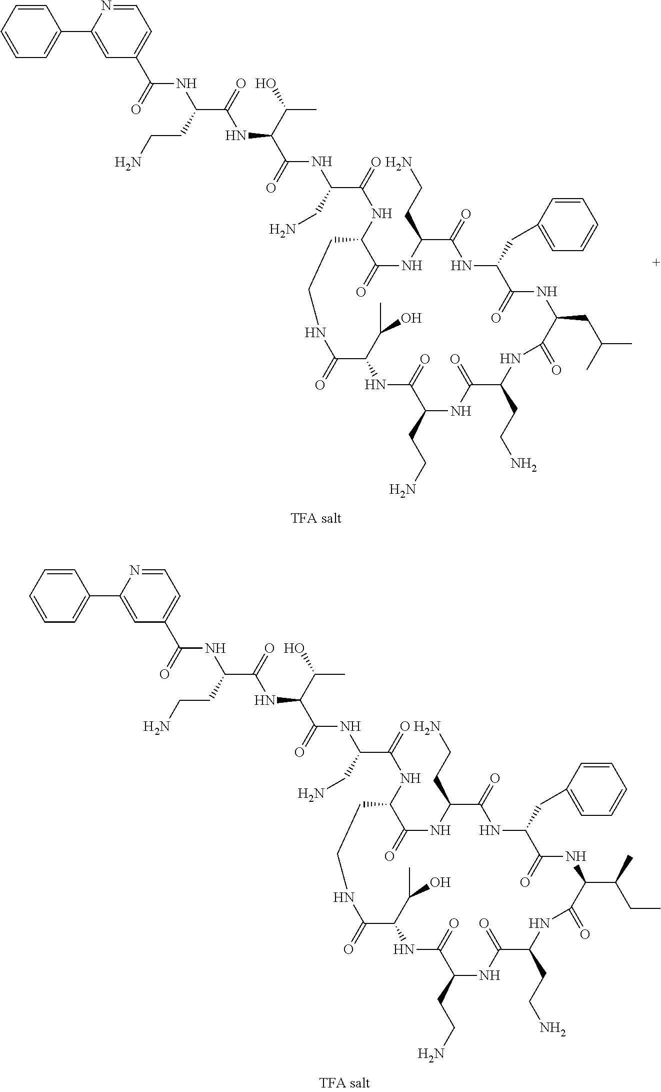

### Example 50C

The product from Example 50B (10 g, 5.78 mmol) in anhydrous DCM (100 mL) under N₂ atmosphere is cooled to 0° C. and trifluoroacetic acid (289 mmol, 22.1 mL) is slowly added. After the addition, the reaction is allowed to warm to ambient temperature and stir for 30-60 min. The mixture is then inversely quenched with methyl t-butyl ether (500 mL). The resulting slurry is vacuum filtrated under nitrogen and the filter cake is dried under nitrogen to give the product as the trifluoroacetate salt.

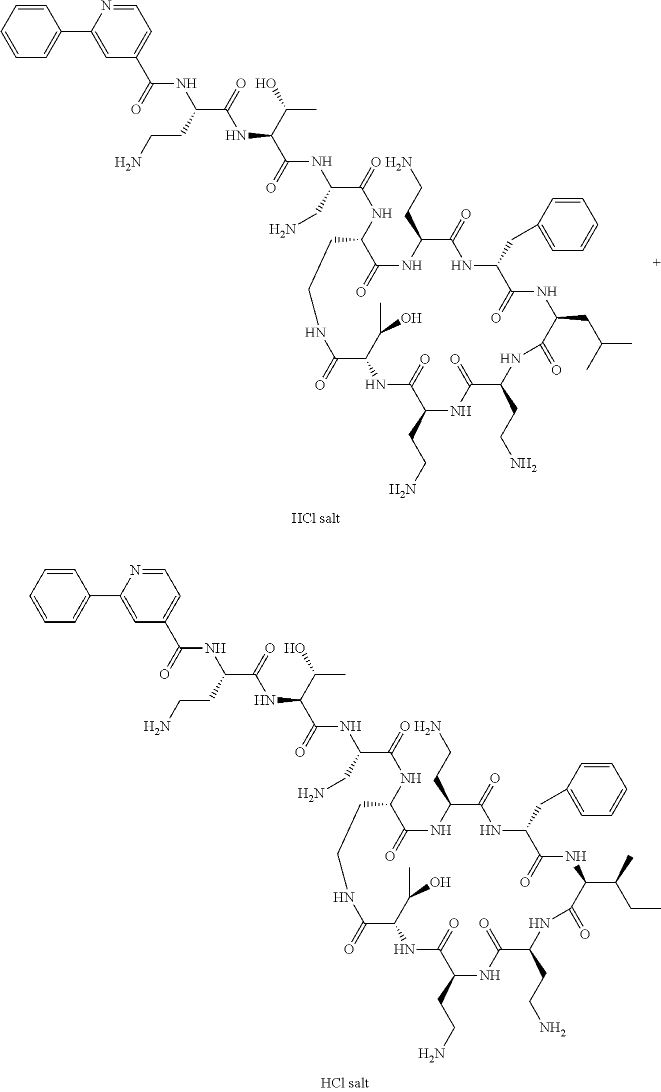

### Example 50D

The trifluoroacetate salt from Example 50C (10 g, 5.56 mmol) is dissolved in anhydrous acetone (50 mL) and 2M HCl solution in diethyl ether (15.5 mL, 31 mmol) is added slowly. After the addition, the mixture is stirred for 10 min, and the solvents are evaporated at room temperature under vacuum to give a residue. Acetone (50 mL) and ethyl acetate (50 mL) are added, and the mixture is stirred for 10 min. The solvents are evaporated under vacuum at room temperature. This is repeated until the residual level of trifluoroacetic acid reaches 0.1 wt % or less (1-2 repetitions) and provides the product of Example X3 as the hydrochloride salt.

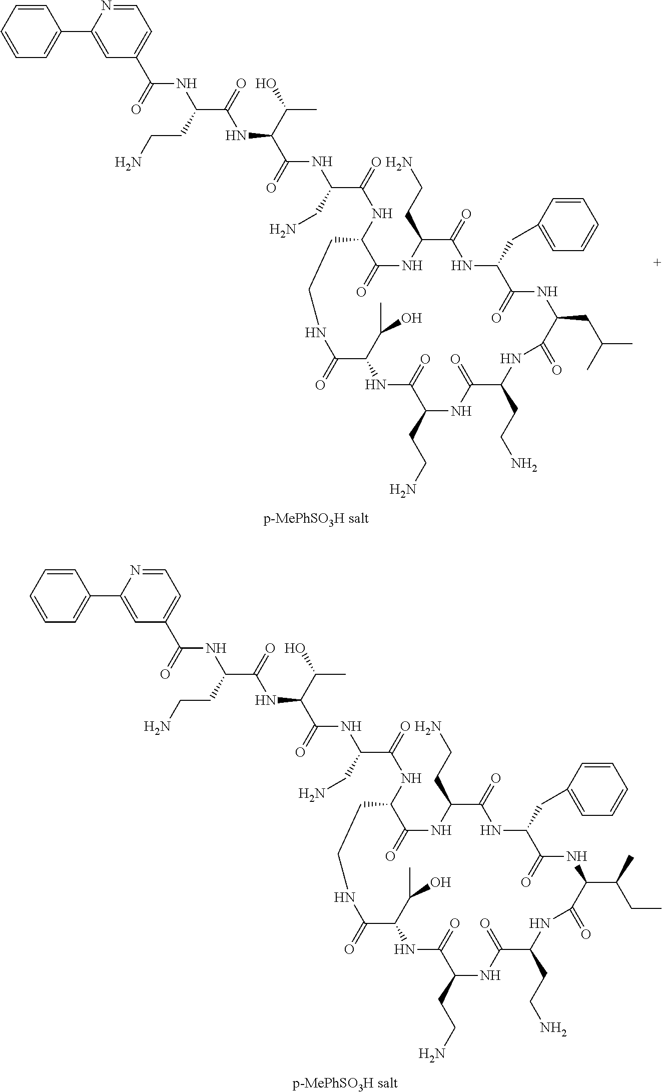

### Example 50E

The trifluoroacetate salt from Example 50C (2 g, 1.11 mmol) is dissolved in anhydrous acetone (10 mL) and p-toluenesulfonic acid monohydrate (1.15 g, 5.5 mmol) is added. After the addition, the mixture is stirred for 30 min and the acetone is evaporated at room temperature under vacuum to give a residue. Acetone (50 mL) and ethyl acetate (50 mL) are added, and the mixture is stirred for 10 min. The solvents are evaporated under vacuum at room temperature. This is repeated until the residual level of trifluoroacetic acid reaches 0.1 wt % or less (1-2 repetitions) and provides the product of Example X3 as the tosylate salt.

### Biological Examples

In order to assess the compounds biological activity, selected in-vitro assays were conducted on selected compounds of Formula I. The specific protocols are described below.

Minimum bactericidal concentration (MBC, mg/L), minimum inhibitory concentration (MIC, mg/L), and human renal proximal tubule epithelial cell line cytotoxicity concentration 50% (hRPTEC, TC50, micromolar) for compounds of Formula I are listed in Table 2. The compounds of Formula I were tested as the corresponding trifluoroacetate salt except for Example 1 which was tested as the sulfate salt. For Table 2 below: PMB=polymyxin B as the sulfate salt (Bedford Laboratories, pharmaceutical grade); PMB1=polymyxin B1 as the trifluoroacetate salt; Nona=polymyxin B nonapeptide as the trifluoroacetate salt. PMB1 and Nona were prepared using the solid phase methodology described in the experimental section herein.

The in vitro antibacterial activity of compounds of Formula I was evaluated by MIC testing according to Clinical and Laboratory Standards Institute (CLSI). See: Clinical and Laboratory Standards Institute. Methods for Dilution Antimicrobial Susceptibility Tests for Bacteria that Grow Aerobically; Approved Standard-Eighth Edition. CLSI document M7-A8 [ISBN 1-56238-689-1]. Clinical and Laboratory Standards Institute, 940 West Valley Road, Suite 1400, Wayne, Pa. 19087-1898 USA, 2006; also Clinical and Laboratory Standards Institute. Performance Standards for Antimicrobial Susceptibility Testing; Twentieth Informational Supplement. CLSI document M100-S20 [ISBN 1-56238-716-2]. Clinical and Laboratory Standards Institute. Methods for Determining Bactericidal Activity of Antimicrobial Agents; Approved Guideline. CLSI document M26-A (ISBN 1-56238-384-1). CLSI, 940 West Valley Road, Suite 1400, Wayne, Pa. 19087-1898 USA, 1999.

The MIC determination is a standard laboratory method for evaluating the antibacterial activity of a compound. The MIC represents the lowest drug concentration that inhibits visible growth of bacteria following overnight incubation. In order to determine the MIC value, a range of drug concentrations (e.g. 0.06 mg/L to 64 mg/L) are incubated with a defined strain of bacteria. Typically, the drug concentration range is broken down into 2-fold increments (e.g. 0.06 mg/L, 0.12 mg/L, 0.25 mg/L, 0.50 mg/L, 1.0 mg/L, etc.) and the various drug concentrations are all individually incubated overnight with approximately the same number of bacteria. The MIC is then determined by visually inspecting the drug effect at each concentration, and identifying the lowest drug concentration that has inhibited bacterial growth as compared to the drug free control. Typically, bacteria continue to grow at drug concentrations lower than the MIC and don't grow at concentrations at and above the MIC.

The MIC values described in Table 2 below were derived from assays wherein each test compound was evaluated in duplicate. In cases where the duplicate values varied by 0-2-fold, the lower of the two values was reported below. Generally speaking, if the duplicate values varied by more than 2-fold, the assay was considered non-valid and was repeated until the variation between duplicate runs was ≦2-fold. In line with the CLSI guidelines referred to above, both control organisms and reference compounds were utilized in each MIC assay to provide proper quality control. MIC values generated with these control organisms and reference compounds were required to fall within a defined range for the assay to be considered valid and be included herein. Those skilled in the art will recognize that MIC values can and do vary from experiment to experiment. Generally speaking, it should be recognized that MIC values often vary +/−2-fold from experiment to experiment. While a single MIC is reported for each compound and each microorganism, the reader should not conclude that each compound was only tested once. Several of the compounds were subjected to multiple tests. The data reported in Table 2 is reflective of the compounds relative activity and different MICs may have been generated on these occasions in line with the guidelines described above.

The following bacterial strains were used in these MIC determinations:

PA-1646 is a polymyxin-resistant clinical isolate of Pseudomonas aeruginosa obtained from JMI labs (www.jmilabs.com);

PA01 is a Pseudomonas aeruginosa strain PA01 described in the following literature references: Antimicrob. Agents Chemother. (2001) 45, 428-432 and FEMS Microbiol. Lett. (2001), 198, 129-134 (www.fems-microbiology.org);

AB-1649 is a polymyxin-resistant clinical isolate of Acinetobacter baumannii obtained from JMI labs (www.jmilabs.com);

AB-3167 is clinical isolate of Acinetobacter baumannii obtained from IHMA (www.ihmainc.com) in 2006;

KP-5016 is an ESBL+clinical isolate of Klebsiella pneumoniae (SHV-5) obtained from JMI labs in 2007;

KP-3700 is an ESBL+clinical isolate of Klebsiella pneumoniae obtained in 2006; and

EC-1 is a mouse virulent strain of Escherichia coli.

### Cytotoxicity Assay

Human Renal Proximal Tubule Epithelial cells (hRPTEC) were obtained from Lonza (Walkersville, Md., USA) and cultured in REGM (Renal Epithelial Cell Basal Medium supplemented with human epithelial growth factor, hydrocortisone, epinephrine, insulin, triiodothyronine, transferrin, gentamycin/amphotericin-B, and fetal bovine serum). The hRPTEC cells were seeded into 96-well plates and incubated at 37° C. in 5% CO₂. Following a growth period of 72 hours, cells were treated with compounds at 100, 67, 44, 30, 20 and 13 micromolar for 24 hours at 37° C. in 5% CO₂. The cell supernatant was then harvested for determination of LDH (lactate dehydrogenase) production. The Cytotoxicity Detection Kit from Roche was used for the measurement of LDH activity released from damaged cells. The Toxic Concentration 50% (TC50, micromolar) values were determined for each compound and compared to the control compounds (polymyxin B was used as a positive control and polymyxin B nonapepetide was used as negative control).

TABLE 2

| Cmpd | PA- | PA- | | AB- | AB- | AB- | KP- | KP- | | |
|---|---|---|---|---|---|---|---|---|---|---|
| or Ex. | 1646 | 1646 | PA01 | 1649 | 1649 | 3167 | 5016 | 3700 | EC-1 | hRPTEC |
| No. | MBC | MIC | MIC | MBC | MIC | MIC | MIC | MIC | MIC | TC50 |
| PMB | 32 | 8 | 0.5 | 8 | 8 | 0.25 | 32 | 0.25 | 0.125 | 20 |
| PMB1 | 16 | 4 | 0.5 | 4 | 4 | 0.25 | 16 | 0.5 | 0.125 | 28 |
| Nona | >64 | >64 | >64 | >64 | >64 | >64 | >64 | >64 | >64 | >100 |
| 1 | 4 | 2 | 0.25 | 1 | 1 | 0.25 | 16 | 0.25 | 0.125 | 95 |
| 2 | 4 | 2 | 0.25 | 0.5 | 0.5 | 0.25 | 8 | 0.25 | 0.125 | 39 |
| 3 | 1 | 1 | 0.5 | 0.5 | 0.5 | 0.25 | 16 | 0.5 | 0.125 | 34 |
| 4 | 16 | 4 | 0.5 | 1 | 1 | 0.25 | 16 | 0.25 | 0.125 | 86 |
| 5 | 4 | 2 | 0.5 | 1 | 1 | 0.125 | 32 | 0.125 | 0.125 | 83 |
| 6 | 16 | 4 | 0.5 | 2 | 2 | 0.25 | 32 | 0.125 | 0.125 | 51 |
| 7 | 8 | 2 | 0.5 | 1 | 1 | 0.25 | 16 | 0.25 | 0.125 | 41 |
| 8 | 4 | 2 | 1 | 1 | 1 | 0.25 | 16 | 0.5 | 0.5 | 32 |
| 9 | 2 | 2 | 0.5 | 2 | 1 | 0.25 | 16 | 0.25 | 0.25 | 24 |
| 10 | 1 | 0.5 | 1 | 1 | 1 | 0.5 | 8 | 0.25 | 0.25 | 14 |
| 11 | 8 | 2 | 0.25 | 4 | 2 | 0.25 | 32 | 0.125 | 0.125 | 84 |
| 12 | 16 | 4 | 1 | 4 | 2 | 0.25 | 64 | 0.25 | 0.25 | 61 |
| 13 | 4 | 2 | 0.5 | 2 | 2 | 0.25 | 16 | 0.5 | 0.25 | 58 |
| 14 | 16 | 4 | 0.5 | 2 | 2 | 0.5 | 32 | 0.5 | 0.5 | 43 |
| 15 | 16 | 4 | 0.5 | 2 | 2 | 0.25 | 32 | 0.25 | 0.125 | 43 |
| 16 | 2 | 1 | 0.5 | 2 | 2 | 0.5 | 16 | 0.25 | 0.25 | 27 |
| 17 | 4 | 2 | 0.5 | 8 | 4 | 0.25 | 16 | 0.25 | 0.25 | 26 |
| 18 | 1 | 1 | 1 | 2 | 2 | 0.5 | 16 | 0.25 | 0.25 | 22 |
| 19 | 2 | 1 | 0.5 | 2 | 2 | 0.25 | 8 | 0.5 | 0.25 | 21 |
| 20 | 1 | 1 | 0.5 | 2 | 2 | 0.25 | 16 | 0.25 | 0.25 | 18 |
| 21 | 2 | 2 | 1 | 2 | 2 | 0.25 | 16 | 0.25 | 0.25 | 15 |
| 22 | 8 | 2 | 0.5 | 2 | 2 | 0.25 | 32 | 0.25 | 0.25 | 78 |
| 23 | 4 | 4 | 0.25 | 8 | 4 | 0.5 | 32 | 0.125 | 0.125 | >100 |
| 24 | 16 | 4 | 1 | 8 | 4 | 0.5 | 64 | 0.25 | 0.25 | >100 |
| 25 | 16 | 4 | 1 | 4 | 4 | 0.25 | 32 | 0.25 | 0.25 | 88 |
| 26 | 8 | 4 | 1 | 4 | 4 | 1 | 32 | 0.25 | 0.25 | 77 |
| 27 | 4 | 2 | 0.5 | 4 | 4 | 0.25 | 32 | 0.25 | 0.125 | 73 |
| 28 | 16 | 4 | 1 | 4 | 4 | 0.25 | 32 | 0.25 | 0.25 | 72 |
| 29 | 32 | 4 | 1 | 4 | 4 | 0.5 | 64 | 0.5 | 0.5 | 70 |
| 30 | 4 | 2 | 0.5 | 4 | 4 | 0.25 | 16 | 0.25 | 0.125 | 56 |
| 31 | 8 | 4 | 1 | 16 | 4 | 0.5 | 64 | 0.5 | 0.5 | 55 |
| 32 | 4 | 2 | 0.5 | 4 | 4 | 0.5 | 16 | 0.5 | 0.25 | 33 |
| 33 | 8 | 2 | 2 | 4 | 4 | 1 | 32 | 1 | 0.5 | 30 |
| 34 | 8 | 2 | 0.5 | 16 | 16 | 0.25 | 16 | 0.125 | 0.125 | 28 |
| 35 | 1 | 1 | 0.5 | 4 | 4 | 0.5 | 16 | 0.25 | 0.25 | 24 |
| 36 | 2 | 2 | 0.5 | 4 | 4 | 0.25 | 32 | 0.25 | 0.5 | 19 |
| 37 | 4 | 2 | 2 | 4 | 4 | 1 | 32 | 1 | 0.5 | 18 |
| 38 | 1 | 1 | 1 | 4 | 4 | 0.25 | 16 | 0.5 | 0.25 | 16 |
| 39 | 4 | 1 | 1 | 4 | 4 | 0.5 | 16 | 0.5 | 0.5 | 20 |
| 40 | 1 | 1 | 0.5 | 4 | 4 | 0.25 | 16 | 0.25 | 0.25 | 15 |
| 41 | 4 | 2 | 0.25 | >64 | 64 | 1 | 64 | 0.5 | 0.25 | 42 |
| 42 | 4 | 1 | 0.5 | 1 | 1 | 0.25 | 16 | 0.25 | 0.25 | 27 |
| 43 | 2 | 1 | 0.5 | 4 | 4 | 0.125 | 8 | 0.125 | 0.125 | 72 |
| 44 | 1 | 1 | 0.5 | 1 | 1 | 0.25 | 16 | 0.25 | 0.25 | 30 |
| 45 | 1 | 0.5 | 0.5 | 1 | 1 | 0.25 | 16 | 0.25 | 0.25 | 28 |
| 46 | ND | 64 | 0.5 | ND | 4 | 1 | 64 | 0.5 | 0.25 | 98 |
| 47 | 2 | 1 | 0.5 | 4 | 4 | 0.5 | 8 | 0.25 | 0.25 | 100 |
| 48 | 2 | 1 | 0.5 | 0.5 | 0.5 | 0.25 | 32 | 0.25 | 0.25 | 33 |
| 49 | 8 | 4 | 0.5 | 0.25 | 0.25 | 0.125 | 16 | 0.25 | 0.125 | >100 |

*ND means not determined.

The data in Table 2 shows that a representative number of compounds of Formula I are, generally, more potent antibacterials than PMB against MDR Gram-negative bacteria and, generally, less cytotoxic than PMB with regard to human renal cells. More specifically, the compounds of Formula I are potent against certain strains of Pseudomonas aeruginosa (PA-1646), Acinetobacter baumannii (PA-1649), and Klebsiella pneumoniae (KP-5016) and can be as much as 5 times less cytotoxic than PMB. For example, the Table 2 data shows that the compound of Example 1 is a more potent inhibitor of growth (MIC) against P. aeruginosa (PA-1646) by 4 fold, A. baumannii (PA-1649) by 8 fold, and K. pneumoniae (KP-5016) by 2 fold with 4-5 fold reduced human renal cytotoxicity than PMB.

The improved antibacterial activity and reduced cytotoxicity derives from, we believe, a structural modification to the PMB template, replacing Dab with Dap at the three-position. For example, the data in Table 2 shows that the compound of Example 43, where Dab is replaced with Dap and the PMB fatty acid chain is left intact, has improved MIC potency against P. aeruginosa (PA-1646) by 8 fold, A. baumannii (PA-1649) by 2 fold, and K. pneumoniae (KP-5016) by 4 fold. In addition to the improved activity against MDR Gram-negative bacteria, Example 43 is 3-4 fold less cytotoxic than PMB. All the compounds of the present invention contain Dap at the three-position instead of Dab.

The present invention also encompasses compounds where the PMB fatty acid chain is replaced with optionally substituted aryl or heteroaryl groups (Examples 1-42 and 44-49). This modification, in combination with Dap at the three-position, is unexpectedly well tolerated with respect to improved antibacterial activity and reduced cytotoxicity as shown by the data in Table 2.

1. A compound of Formula I 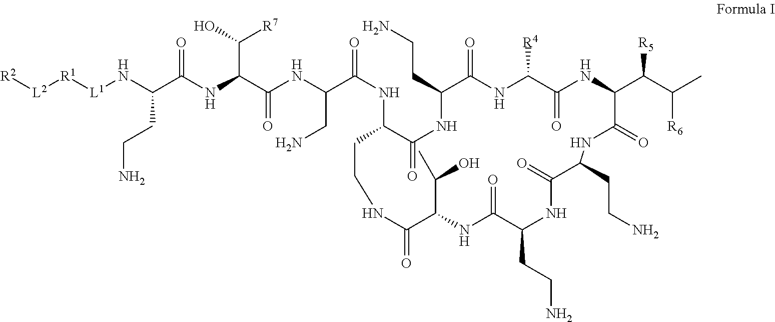
or a pharmaceutically acceptable salt thereof
wherein
L¹ is a bond, carbonyl, sulfonyl, —NHC(O)—, or —OC(O)—;
R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;
L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;
R² is absent, (C₆-C₁₀)aryl, or (C₅-C₁₂)heteroaryl;
R³ is H or (C₁-C₃)alkyl;
R⁴ is (C₁-C₆)alkyl or (C₆-C₁₀)aryl(C₁-C₆)alkyl;
R⁵ is H or CH₃;
R⁶ is H or CH₃;
wherein R⁶ is H when R⁵ is CH₃ and wherein R⁶ is CH₃ when R⁵ is H;
R⁷ is H or CH₃;
n is 0, 1, 2, or 3; and
p is 0, 1, 2, or 3.

2. A compound of Formula II 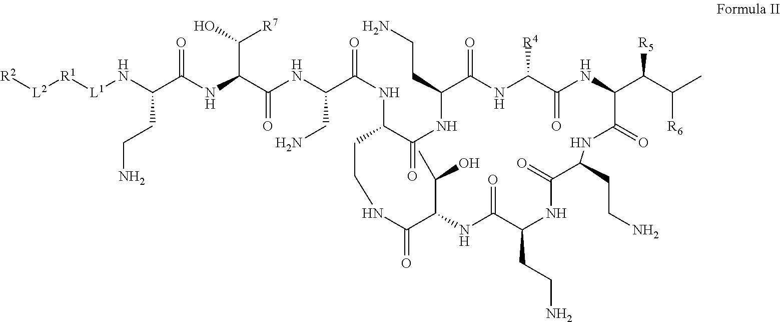
or a pharmaceutically acceptable salt thereof
wherein
L¹ is a bond, carbonyl, sulfonyl, —NHC(O)—, or —OC(O)—;
R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;
L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;
R² is absent, (C₆-C₁₀)aryl, or (C₅-C₁₂)heteroaryl;
R³ is H or (C₁-C₃)alkyl;
R⁴ is (C₁-C₆)alkyl or (C₆-C₁₀)aryl(C₁-C₆)alkyl;
R⁵ is H or CH₃;
R⁶ is H or CH₃;
wherein R⁶ is H when R⁵ is CH₃ and wherein R⁶ is CH₃ when R⁵ is H;
R⁷ is H or CH₃;
n is 0, 1, 2, or 3; and
p is 0, 1, 2, or 3.

3. The compound according to claim 2, or a
pharmaceutically acceptable salt thereof, wherein
L¹ is a bond, carbonyl, sulfonyl, or —NHC(O)—;
R¹ is (C₁-C₁₀)alkyl, (C₆-C₁₀)aryl, or (C₅-C₁₂)heteroaryl;
L² is absent, a bond, or —(CH₂)ₙO(CH₂)ₚ—;
R² is absent, (C₆-C₁₀)aryl, or (C₅-C₁₂)heteroaryl;
R⁴ is benzyl;
R⁵ is H or CH₃;
R⁶ is H or CH₃;
wherein R⁶ is H when R⁵ is CH₃ and wherein R⁶ is CH₃ when R⁵ is H;
R⁷ is CH₃;
n is 0; and
p is 0.

4. The compound according to claim 2, or a
pharmaceutically acceptable salt thereof, wherein
L¹ is carbonyl;
R⁴ is benzyl;
R⁵ is H or CH₃;
R⁶ is H or CH₃;
wherein R⁶ is H when R⁵ is CH₃ and wherein R⁶ is CH₃ when R⁵ is H; and
R⁷ is CH₃.

5. The compound according to claim 2, or a
pharmaceutically acceptable salt thereof, wherein
L¹ is carbonyl;
R¹ is (C₅-C₁₂)heteroaryl;
R⁴ is benzyl;
R⁵ is H or CH₃;
R⁶ is H or CH₃;
wherein R⁶ is H when R⁵ is CH₃ and wherein R⁶ is CH₃ when R⁵ is H; and
R⁷ is CH₃.

6. The compound according to claim 2, or a
pharmaceutically acceptable salt thereof, wherein
L¹ is carbonyl;
R¹ is (C₅-C₁₂)heteroaryl wherein the (C₅-C₁₂)heteroaryl is pyridazinyl or pyridinyl optionally substituted with 1 substituent that is (C₁-C₆)alkoxy;
L² is a bond;
R² is (C₆-C₁₀)aryl;
R⁴ is benzyl;
R⁵ is H; and
R⁶ is CH₃.

7. The compound according to claim 2, or a
pharmaceutically acceptable salt thereof, wherein
L¹ is carbonyl;
R¹ is (C₅-C₁₂)heteroaryl wherein the (C₅-C₁₂)heteroaryl is pyridazinyl or pyridinyl optionally substituted with 1 substituent that is (C₁-C₆)alkoxy;
L² is a bond;
R² is phenyl optionally substituted with 1 or 2 substituents that are independently (C₁-C₆)alkoxy, cyano, or halogen;
R⁴ is benzyl;
R⁵ is H;
R⁶ is CH₃; and
R⁷ is CH₃.

8. The compound according to claim 2 that is 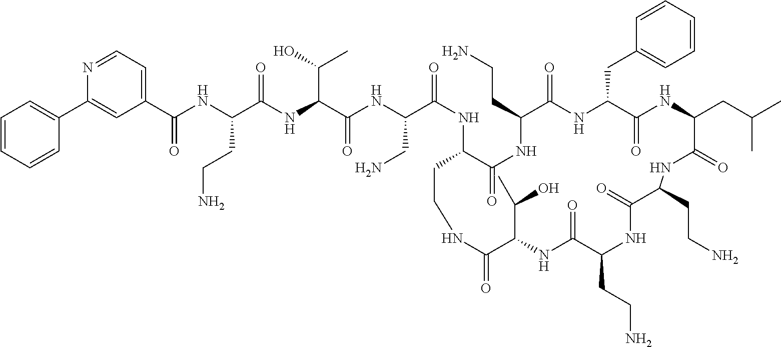
or a pharmaceutically acceptable salt thereof.

9. The compound according to claim 2 that is 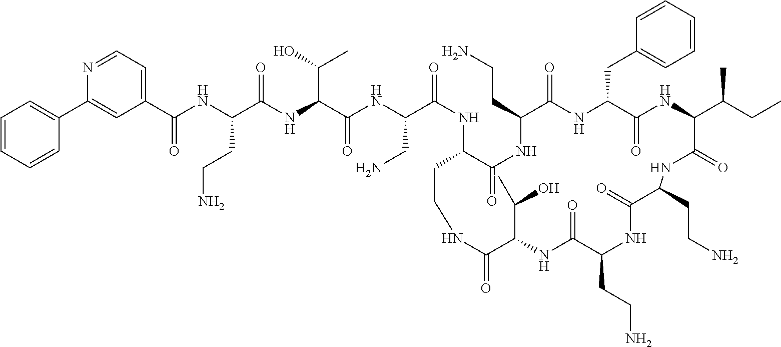
or a pharmaceutically acceptable salt thereof.

10. The compound according to claim 2 that is 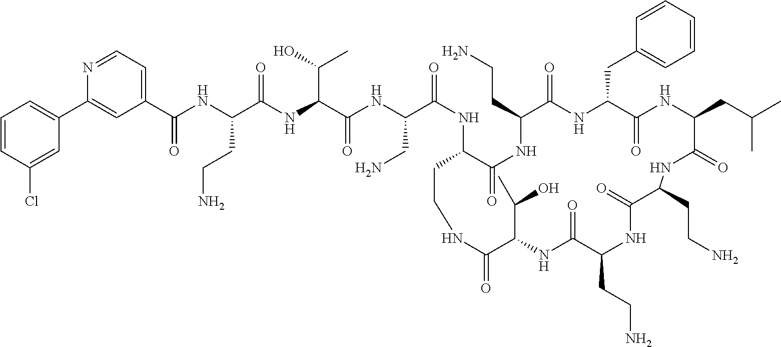
or a pharmaceutically acceptable salt thereof.

11. The compound according to claim 2 that is 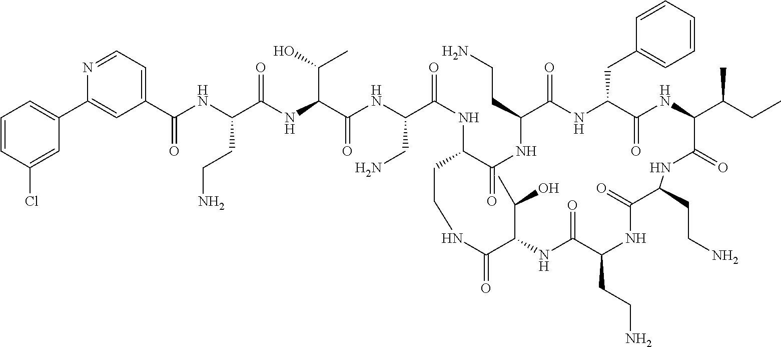
or a pharmaceutically acceptable salt thereof.

12. The compound according to claim 2, or a
pharmaceutically acceptable salt thereof, wherein
L¹ is carbonyl;
R¹ is (C₆-C₁₀)aryl;
R⁴ is benzyl;
R⁵ is H or CH₃;
R⁶ is H or CH₃;
wherein R⁶ is H when R⁵ is CH₃ and wherein R⁶ is CH₃ when R⁵ is H; and
R⁷ is CH₃.

13. The compound according to claim 2, or a
pharmaceutically acceptable salt thereof, wherein
L¹ is carbonyl;
R¹ is phenyl optionally substituted with 1 substituent that is (C₁-C₆)alkoxy, cyano, or halogen;
L² is a bond;
R² is (C₆-C₁₀)aryl;
R⁴ is benzyl;
R⁵ is H;
R⁶ is CH₃; and
R⁷ is CH₃.

14. The compound according to claim 2, or a
pharmaceutically acceptable salt thereof, wherein
L¹ is carbonyl;
R¹ is phenyl optionally substituted with 1 substituent that is (C₁-C₆)alkoxy, cyano, or halogen;
L² is a bond;
R² is phenyl optionally substituted with 1 substituent that is (C₁-C₆)alkoxy, (C₁-C₆)alkyl, cyano, or halogen;
R⁴ is benzyl;
R⁵ is H;
R⁶ is CH₃; and
R⁷ is CH₃.

15. A compound of Formula III 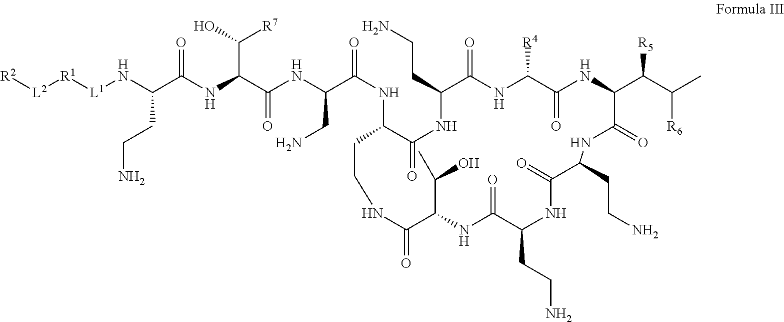
or a pharmaceutically acceptable salt thereof
wherein
L¹ is a bond, carbonyl, sulfonyl, —NHC(O)—, or —OC(O)—;
R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;
L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;
R² is absent, (C₆-C₁₀)aryl, or (C₅-C₁₂)heteroaryl;
R³ is H or (C₁-C₃)alkyl;
R⁴ is (C₁-C₆)alkyl or (C₆-C₁₀)aryl(C₁-C₆)alkyl;
R⁵ is H or CH₃;
R⁶ is H or CH₃;
wherein R⁶ is H when R⁵ is CH₃ and wherein R⁶ is CH₃ when R⁵ is H;
R⁷ is H or CH₃;
n is 0, 1, 2, or 3; and
p is 0, 1, 2, or 3.

16. A compound of Formula IV 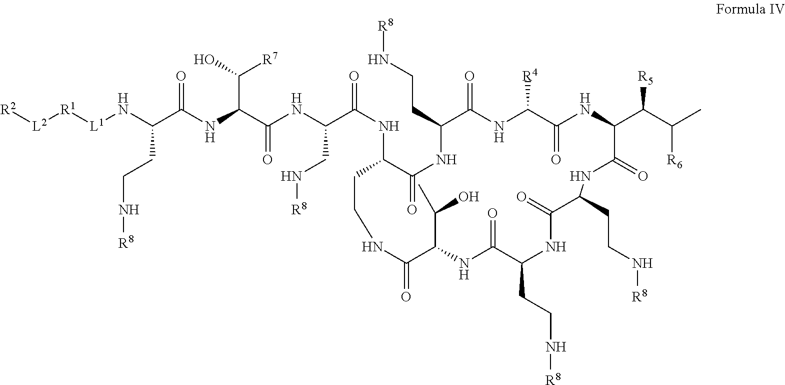
or a pharmaceutically acceptable salt thereof
wherein
L¹ is a bond, carbonyl, sulfonyl, —NHC(O)—, or —OC(O)—;
R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;
L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;
R² is absent, (C₆-C₁₀)aryl, or (C₅-C₁₂)heteroaryl;
R³ is H or (C₁-C₃)alkyl;
R⁴ is (C₁-C₆)alkyl or (C₆-C₁₀)aryl(C₁-C₆)alkyl;
R⁵ is H or CH₃;
R⁶ is H or CH₃;
wherein R⁶ is H when R⁵ is CH₃ and wherein R⁶ is CH₃ when R⁵ is H;
R⁷ is H or CH₃;
R⁸ is (C₁-C₁₀)alkylcarbonyl, (C₁-C₁₀)alkyl-O-carbonyl, (C₆-C₁₀)aryl-O-carbonyl, or —CH₂—SO₂OH;
n is 0, 1, 2, or 3; and
p is 0, 1, 2, or 3.

17. A compound of Formula V 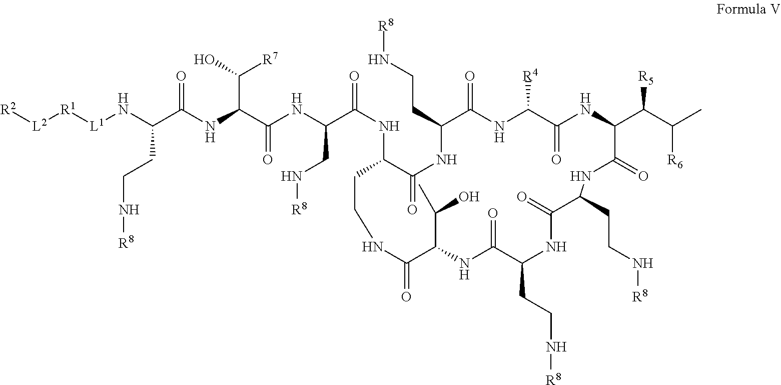
or a pharmaceutically acceptable salt thereof
wherein
L¹ is a bond, carbonyl, sulfonyl, —NHC(O)—, or —OC(O)—;
R¹ is (C₁-C₁₀)alkyl, (C₆-C₁₀)aryl, (C₆-C₁₀)aryl(C₂-C₄)alkenyl, (C₅-C₁₂)heteroaryl, or (C₅-C₁₂)heteroaryl(C₂-C₄)alkenyl;
L² is absent, a bond, —(CH₂)ₙ—, —(CH₂)ₙO(CH₂)ₚ—, —(CH₂)ₙS(CH₂)ₚ—, or —(CH₂)ₙNR³(CH₂)ₚ—;
R² is absent, (C₆-C₁₀)aryl, or (C₅-C₁₂)heteroaryl;
R³ is H or (C₁-C₃)alkyl;
R⁴ is (C₁-C₆)alkyl or (C₆-C₁₀)aryl(C₁-C₆)alkyl;
R⁵ is H or CH₃;
R⁶ is H or CH₃;
wherein R⁶ is H when R⁵ is CH₃ and wherein R⁶ is CH₃ when R⁵ is H;
R⁷ is H or CH₃;
R⁸ is (C₁-C₁₀)alkylcarbonyl, (C₁-C₁₀)alkyl-O-carbonyl, (C₆-C₁₀)aryl-O-carbonyl, or —CH₂—SO₂OH;
n is 0, 1, 2, or 3; and
p is 0, 1, 2, or 3.

18. A pharmaceutical composition comprising a compound according to any of claims 1, 2, 15, 16 and 17 or a pharmaceutically acceptable salt thereof in admixture with at least one pharmaceutically acceptable excipient.

19. A method for treating bacterial infections in a mammal comprising administering to the mammal in need of such treatment a therapeutically effect amount of a compound according to any one of claims 1, 2, 15, 16 and 17 or a pharmaceutically acceptable salt thereof.

20. The method according to claim 19 wherein the bacterial infections are Gram-negative bacterial infections.

21. The method according to claim 20 wherein the Gram-negative infections are associated with Pseudomonas aeruginosa, Acinetobacter baumannii, and Klebsiella pneumoniae.

22. The method according to claim 19 wherein the bacterial infections are nosocomial pneumonia, urinary tract infections, bacteremia, sepsis, skin and soft tissue infections, surgical infections, intraabdominal infections, lung infections other than nosocomial pneumonia, Helicobacter pylori, endocarditis, diabetic foot infections, osteomyelitis, or central nervous system infections.
